SEQUENCE LISTING (16 sequences)

SEQ ID NO: 1 - DNA, 2121 bp, synthetic construct
  source 1..2121: mol_type = other DNA
atgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttaagagatactgaacagatcttcagagaaggtccggttgaaattggtctcccatgctccatcaaaaatgaagcaaggccgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaataaaggccttaaagagagatgtggtcttgatggacgatgatgttgagtgcacgatggtagagaagagagttctttccttggcctgggagcatccgtttctgacgcacatgttttgtacattccagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaaccccgggatggagcggctgatatcctga

SEQ ID NO: 2 - protein, 706 aa, synthetic construct
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKRDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 3 - DNA, 2121 bp, Homo sapiens
  source 1..2121: mol_type = genomic DNA
atgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttaagagatactgaacagatcttcagagaaggtccggttgaaattggtctcccatgctccatcaaaaatgaagcaaggccgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaataaaggccttaaagaaagatgtggtcttgatggacgatgatgttgagtgcacgatggtagagaagagagttctttccttggcctgggagcatccgtttctgacgcacatgttttgtacattccagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaaccccgggatggagcggctgatatcctga

SEQ ID NO: 4 - protein, 706 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 5 - DNA, 2124 bp, synthetic construct
  source 1..2124: mol_type = other DNA
atgtcaccgtttcttcgaatcggtttatccaactttgactgtgggacctgccaagcttgtcagggagaggcagtgaacccctactgcgctgtgcttgtcaaagagtatgtggaatcagaaaatgggcagatgtacatccagaaaaagccaaccatgtacccaccttgggacagcacctttgacgcccacattaacaagggaagggtgatgcagatcatcgtgaagggcaagaatgtagacctcatctcagaaacaaccgtggaactctactccctggcggagagatgccgcaagaacaatgggcggacagaaatatggttagagctgaaacctcaaggccgaatgctaatgaatgcaagatactttctggaaatgagtgacacaaaggacatgagtgagtttgagaatgaaggattctttgcactgcatcagcgccgaggagccatcaaacaggccaaagtccaccatgtcaagtgtcacgagttcacggccacctttttccctcaacccacattttgctctgtctgccatgaatttgtctggggcctgaacaagcagggttaccagtgccgacagtgtaatgcagcgattcacaagaagtgcattgataaagtgatagccaagtgcacaggatccgcaatcaatagccgagaaaccatgttccataaggagagattcaagatcgacatgccacacagattcaaagtctacaactacaagagtccaaccttctgtgagcactgtggtaccctgctctgggggctggcgaggcaaggactcaaatgtgatgcatgtggcatgaacgtccaccaccgatgccagacaaaggttgccaatctttgtggtataaaccagaagctaatggctgaagcactagcgatgattgaaagcacccaacaggctcgctccttacgagattcagaacacatcttccgagaaggcccagttgaaattggtctcccatgctccaccaaaaacgaaaccaggccaccatgcgtaccaacacctgggaaaagagaaccccagggcatttcctgggattcccctttggatgggtcaaataaatcggccggtcctcctgaacccgaagtgagcatgcgcaggacttcactgcagctgaaactgaagatcgatgacttcatcctgcacaagatgttgggaaaaggaagttttggcaaggtcttcctggcagagttcaagagaaccaatcagtttttcgcaataaaagccttaaagagagatgtggtgttgatggatgatgacgtcgagtgtacaatggtggaaaagagggttctgtccttggcatgggagcatccatttctaacacacatgttctgcacattccagaccaaggaaaatctctttttcgtgatggagtatctcaatggaggcgacttaatgtaccacatccaaagttgccacaaatttgatctttccagagccacgttttatgctgctgaggtcatccttggtctgcagttccttcattccaaaggaattgtctacagggacctgaagcttgataatatcctgttagacagagatggacatatcaaaatagcagactttgggatgtgcaaagagaacatgctaggagatgcgaagacaaatactttctgtggaactcctgactacattgctccggagatcttgctgggtcagaagtacaaccattccgtcgactggtggtccttcggggtgctcgtttatgagatgctgattggccagtcccccttccacgggcaggacgaggaggagctgttccactccatccgcatggacaaccccttctacccgaggtggctcgaaagggaggccaaggaccttctagtgaagctttttgtgagagaacctgagaagaggctgggagtgagaggagacatccgccagcatcctttgtttcgagagatcaactgggaagagcttgaaaggaaagagattgacccacccttcagaccaaaagtgaaatcaccatatgactgtagcaatttcgacaaggaattcctaagtgagaaaccccggctatcattcgccgacagagcactcatcaacagcatggaccagaacatgttcagcaacttttccttcattaacccagggatggagactctcatttgctcctga

SEQ ID NO: 6 - protein, 707 aa, synthetic construct
MSPFLRIGLSNFDCGTCQACQGEAVNPYCAVLVKEYVESENGQMYIQKKPTMYPPWDSTFDAHINKGRVMQIIVKGKNVDLISETTVELYSLAERCRKNNGRTEIWLELKPQGRMLMNARYFLEMSDTKDMSEFENEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARSLRDSEHIFREGPVEIGLPCSTKNETRPPCVPTPGKREPQGISWDSPLDGSNKSAGPPEPEVSMRRTSLQLKLKIDDFILHKMLGKGSFGKVFLAEFKRTNQFFAIKALKRDVVLMDDDVECTMVEKRVLSLAWEHPFLTHMFCTFQTKENLFFVMEYLNGGDLMYHIQSCHKFDLSRATFYAAEVILGLQFLHSKGIVYRDLKLDNILLDRDGHIKIADFGMCKENMLGDAKTNTFCGTPDYIAPEILLGQKYNHSVDWWSFGVLVYEMLIGQSPFHGQDEEELFHSIRMDNPFYPRWLEREAKDLLVKLFVREPEKRLGVRGDIRQHPLFREINWEELERKEIDPPFRPKVKSPYDCSNFDKEFLSEKPRLSFADRALINSMDQNMFSNFSFINPGMETLICS

SEQ ID NO: 7 - DNA, 2124 bp, Mus sp.
  source 1..2124: mol_type = genomic DNA
atgtcaccgtttcttcgaatcggtttatccaactttgactgtgggacctgccaagcttgtcagggagaggcagtgaacccctactgcgctgtgcttgtcaaagagtatgtggaatcagaaaatgggcagatgtacatccagaaaaagccaaccatgtacccaccttgggacagcacctttgacgcccacattaacaagggaagggtgatgcagatcatcgtgaagggcaagaatgtagacctcatctcagaaacaaccgtggaactctactccctggcggagagatgccgcaagaacaatgggcggacagaaatatggttagagctgaaacctcaaggccgaatgctaatgaatgcaagatactttctggaaatgagtgacacaaaggacatgagtgagtttgagaatgaaggattctttgcactgcatcagcgccgaggagccatcaaacaggccaaagtccaccatgtcaagtgtcacgagttcacggccacctttttccctcaacccacattttgctctgtctgccatgaatttgtctggggcctgaacaagcagggttaccagtgccgacagtgtaatgcagcgattcacaagaagtgcattgataaagtgatagccaagtgcacaggatccgcaatcaatagccgagaaaccatgttccataaggagagattcaagatcgacatgccacacagattcaaagtctacaactacaagagtccaaccttctgtgagcactgtggtaccctgctctgggggctggcgaggcaaggactcaaatgtgatgcatgtggcatgaacgtccaccaccgatgccagacaaaggttgccaatctttgtggtataaaccagaagctaatggctgaagcactagcgatgattgaaagcacccaacaggctcgctccttacgagattcagaacacatcttccgagaaggcccagttgaaattggtctcccatgctccaccaaaaacgaaaccaggccaccatgcgtaccaacacctgggaaaagagaaccccagggcatttcctgggattcccctttggatgggtcaaataaatcggccggtcctcctgaacccgaagtgagcatgcgcaggacttcactgcagctgaaactgaagatcgatgacttcatcctgcacaagatgttgggaaaaggaagttttggcaaggtcttcctggcagagttcaagagaaccaatcagtttttcgcaataaaagccttaaagaaagatgtggtgttgatggatgatgacgtcgagtgtacaatggtggaaaagagggttctgtccttggcatgggagcatccatttctaacacacatgttctgcacattccagaccaaggaaaatctctttttcgtgatggagtatctcaatggaggcgacttaatgtaccacatccaaagttgccacaaatttgatctttccagagccacgttttatgctgctgaggtcatccttggtctgcagttccttcattccaaaggaattgtctacagggacctgaagcttgataatatcctgttagacagagatggacatatcaaaatagcagactttgggatgtgcaaagagaacatgctaggagatgcgaagacaaatactttctgtggaactcctgactacattgctccggagatcttgctgggtcagaagtacaaccattccgtcgactggtggtccttcggggtgctcgtttatgagatgctgattggccagtcccccttccacgggcaggacgaggaggagctgttccactccatccgcatggacaaccccttctacccgaggtggctcgaaagggaggccaaggaccttctagtgaagctttttgtgagagaacctgagaagaggctgggagtgagaggagacatccgccagcatcctttgtttcgagagatcaactgggaagagcttgaaaggaaagagattgacccacccttcagaccaaaagtgaaatcaccatatgactgtagcaatttcgacaaggaattcctaagtgagaaaccccggctatcattcgccgacagagcactcatcaacagcatggaccagaacatgttcagcaacttttccttcattaacccagggatggagactctcatttgctcctga

SEQ ID NO: 8 - protein, 707 aa, Mus sp.
MSPFLRIGLSNFDCGTCQACQGEAVNPYCAVLVKEYVESENGQMYIQKKPTMYPPWDSTFDAHINKGRVMQIIVKGKNVDLISETTVELYSLAERCRKNNGRTEIWLELKPQGRMLMNARYFLEMSDTKDMSEFENEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARSLRDSEHIFREGPVEIGLPCSTKNETRPPCVPTPGKREPQGISWDSPLDGSNKSAGPPEPEVSMRRTSLQLKLKIDDFILHKMLGKGSFGKVFLAEFKRTNQFFAIKALKKDVVLMDDDVECTMVEKRVLSLAWEHPFLTHMFCTFQTKENLFFVMEYLNGGDLMYHIQSCHKFDLSRATFYAAEVILGLQFLHSKGIVYRDLKLDNILLDRDGHIKIADFGMCKENMLGDAKTNTFCGTPDYIAPEILLGQKYNHSVDWWSFGVLVYEMLIGQSPFHGQDEEELFHSIRMDNPFYPRWLEREAKDLLVKLFVREPEKRLGVRGDIRQHPLFREINWEELERKEIDPPFRPKVKSPYDCSNFDKEFLSEKPRLSFADRALINSMDQNMFSNFSFINPGMETLICS

SEQ ID NO: 9 - DNA, 1248 bp, Homo sapiens
  source 1..1248: mol_type = genomic DNA
atggattttgacaagaaaggagggaaaggggagacggaggagggccggagaatgtccaaggccggcgggggccggagcagccacggcatccggagctcggggaccagctcgggggtcctgatggtgggccccaacttccgcgtcggcaagaagatcggctgcggcaacttcggggagctccgcctaggaaagaatctctatacaaatgaatacgtggctatcaaattggagccgatcaagtcccgggccccgcagctgcacctggagtaccggttctacaagcagctcagcgccacagagggcgtccctcaggtctactacttcggtccgtgcgggaagtacaacgccatggtgctggagctgctggggcccagcctggaggacctgttcgacctgtgcgaccggaccttcacgctcaagacggtgctgatgatcgccatccagctgatcacgcgcatggagtatgtgcacaccaagagcctaatctaccgggacgtgaagcccgagaacttcctggtgggccgcccggggaccaagcggcagcatgccatccacatcatcgacttcgggctggccaaggagtacatcgaccccgagaccaagaagcacatcccgtaccgcgagcacaagagcctgacgggcacggcgcgctacatgagcatcaacacgcacctgggcaaggagcagagccgccgcgacgacctggaggcgctgggccacatgttcatgtacttcctgcgcggcagcctcccctggcaggggctcaaggccgacacgctcaaggagcggtaccagaagatcggggacaccaaacgcgccacgcccatcgaggtgctctgcgagaacttcccagaggagatggccacgtacctgcgctatgtgcggcgcctggacttcttcgagaagcccgactatgactacctgcggaagctcttcaccgacctcttcgaccgcagtggcttcgtgttcgactatgagtacgactgggccgggaagcccctgccgacccccatcggcaccgtccacaccgacctgccctcccagcctcagctccgggacaaaacccagccgcacagcaaaaaccaggcgttgaactccaccaacggggagctgaatgcggacgaccccacggccggccactccaacgccccgatcacagcgcctgcagaggtggaggtggccgatgaaaccaaatgctgctgtttcttcaagaggagaaagagaaaatcgctgcagcgacacaagtga

SEQ ID NO: 10 - protein, 682 aa, Mus musculus
MSSGTMKFNGYLRVRIGEAVGLQPTRWSLRHSLFKKGHQLLDPYLTVSVDQVRVGQTSTKQKTNKPTYNEEFCANVTDGGHLELAVFHETPLGYDHFVANCTLQFQELLRTAGTSDTFEGWVDLEPEGKVFVVITLTGSFTEATLQRDRIFKHFTRKRQRAMRRRVHQVNGHKFMATYLRQPTYCSHCREFIWGVFGKQGYQCQVCTCVVHKRCHHLIVTACTCQNNINKVDAKIAEQRFGINIPHKFNVHNYKVPTFCDHCGSLLWGIMRQGLQCKICKMNVHIRCQANVAPNCGVNAVELAKTLAGMGLQPGNISPTSKLISRSTLRRQGKEGSKEGNGIGVNSSSRFGIDNFEFIRVLGKGSFGKVMLARIKETGELYAVKVLKKDVILQDDDVECTMTEKRILSLARNHPFLTQLFCCFQTPDRLFFVMEFVNGGDLMFHIQKSRRFDEARARFYAAEIISALMFLHEKGIIYRDLKLDNVLLDHEGHCKLADFGMCKEGICNGVTTATFCGTPDYIAPEILQEMLYGPAVDWWAMGVLLYEMLCGHAPFEAENEDDLFEAILNDEVVYPTWLHEDATGILKSFMTKNPTMRLGSLTQGGEHEILRHPFFKEIDWAQLNHRQLEPPFRPRIKSREDVSNFDPDFIKEEPVLTPIDEGHLPMINQDEFRNFSYVSPELQ

SEQ ID NO: 11 - protein, 682 aa, Homo sapiens
MSSGTMKFNGYLRVRIGEAVGLQPTRWSLRHSLFKKGHQLLDPYLTVSVDQVRVGQTSTKQKTNKPTYNEEFCANVTDGGHLELAVFHETPLGYDHFVANCTLQFQELLRTTGASDTFEGWVDLEPEGKVFVVITLTGSFTEATLQRDRIFKHFTRKRQRAMRRRVHQINGHKFMATYLRQPTYCSHCREFIWGVFGKQGYQCQVCTCVVHKRCHHLIVTACTCQNNINKVDSKIAEQRFGINIPHKFSIHNYKVPTFCDHCGSLLWGIMRQGLQCKICKMNVHIRCQANVAPNCGVNAVELAKTLAGMGLQPGNISPTSKLVSRSTLRRQGKESSKEGNGIGVNSSNRLGIDNFEFIRVLGKGSFGKVMLARVKETGDLYAVKVLKKDVILQDDDVECTMTEKRILSLARNHPFLTQLFCCFQTPDRLFFVMEFVNGGDLMFHIQKSRRFDEARARFYAAEIISALMFLHDKGIIYRDLKLDNVLLDHEGHCKLADFGMCKEGICNGVTTATFCGTPDYIAPEILQEMLYGPAVDWWAMGVLLYEMLCGHAPFEAENEDDLFEAILNDEVVYPTWLHEDATGILKSFMTKNPTMRLGSLTQGGEHAILRHPFFKEIDWAQLNHRQIEPPFRPRIKSREDVSNFDPDFIKEEPVLTPIDEGHLPMINQDEFRNFSYVSPELQ

SEQ ID NO: 12 - protein, 705 aa, Mus musculus
MSPFLRIGLSNFDCGTCQACQGEAVNPYCAVLVKEYVESENGQMYIQKKPTMYPPWDSTFDAHINKGRVMQIIVKGKNVDLISETTVELYSLAERCRKNNGRTEIWLELKPQGRMLMNARYFLEMSDTKDMSEFENEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARSLRDSEHIFREGPVEIGLPCSTKNETRPPCVPTPGKREPQGISWDSPLDGSNKSAGPPEPEVSMRRTSLQLKLKIDDFILHKMLGKGSFGKVFLAEFKRTNQFFAIKALKKDVVLMDDDVECTMVEKRVLSLAWEHPFLTHMFCTFQTKENLFFVMEYLNGGDLMYHIQSCHKFDLSRATFYAAEVILGLQFLHSKGIVYRDLKLDNILLDRDGHIKIADFGMCKENMLGDAKTNTFCGTPDYIAPEILLGQKYNHSVDWWSFGVLVYEMLIGQSPFHGQDEEELFHSIRMDNPFYPRWLEREAKDLLVKLFVREPEKRLGVRGDIRQHPLFREINWEELERKEIDPPFRPKVKSPYDCSNFDKEFLSEKPRLSFADRALINSMDQNMFSNFSFINPGMETLI

SEQ ID NO: 13 - protein, 705 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLI

SEQ ID NO: 14 - protein, 415 aa, Mus musculus
MDFDKKGGKGELEEGRRMSKTGTSRSNHGVRSSGTSSGVLMVGPNFRVGKKIGCGNFGELRLGKNLYTNEYVAIKLEPIKSRAPQLHLEYRFYKQLSTTEGVPQVYYFGPCGKYNAMVLELLGPSLEDLFDLCDRTFTLKTVLMIAIQLITRMEYVHTKSLIYRDVKPENFLVGRPGSKRQHSIHIIDFGLAKEYIDPETKKHIPYREHKSLTGTARYMSINTHLGKEQSRRDDLEALGHMFMYFLRGSLPWQGLKADTLKERYQKIGDTKRATPIEVLCESFPEEMATYLRYVRRLDFFEKPDYDYLRKLFTDLFDRSGYVFDYEYDWAGKPLPTPIGTVHPDVPSQPPHRDKAQLHTKNQALNSTNGELNTDDPTAGHSNAPIAAPAEVEVADETKCCCFFKRRKRKSLQRHK

SEQ ID NO: 15 - protein, 415 aa, Homo sapiens
MDFDKKGGKGETEEGRRMSKAGGGRSSHGIRSSGTSSGVLMVGPNFRVGKKIGCGNFGELRLGKNLYTNEYVAIKLEPIKSRAPQLHLEYRFYKQLSATEGVPQVYYFGPCGKYNAMVLELLGPSLEDLFDLCDRTFTLKTVLMIAIQLITRMEYVHTKSLIYRDVKPENFLVGRPGTKRQHAIHIIDFGLAKEYIDPETKKHIPYREHKSLTGTARYMSINTHLGKEQSRRDDLEALGHMFMYFLRGSLPWQGLKADTLKERYQKIGDTKRATPIEVLCENFPEEMATYLRYVRRLDFFEKPDYDYLRKLFTDLFDRSGFVFDYEYDWAGKPLPTPIGTVHTDLPSQPQLRDKTQPHSKNQALNSTNGELNADDPTAGHSNAPITAPAEVEVADETKCCCFFKRRKRKSLQRHK

SEQ ID NO: 16 - protein, 9 aa, unidentified
LAGLIDADG

(19) United States
(12) Patent Application Publication
(10) Pub. No.: US 2026/0146240 A1
(43) Pub. Date: May 28, 2026

(54) COMPOSITIONS AND METHODS FOR TREATING OR PREVENTING T CELL EXHAUSTION

(71) Applicant: Salk Institute for Biological Studies, La Jolla, CA (US)

(72) Inventors: Thomas H. Mann, San Diego, CA (US); Susan M. Kaech, Del Mar, CA (US)

(21) Appl. No.: 19/267,327

(22) Filed: Jul. 11, 2025

Related U.S. Application Data
(60) Provisional application No. 63/670,716, filed on Jul. 12, 2024.

(51) Int. Cl. C12N 9/12; A61K 40/11; A61K 40/30; A61P 31/14; A61P 35/00

(52) U.S. Cl. CPC C12N 9/12; A61K 40/11; A61K 40/30; A61P 31/14; A61P 35/00; C12Y 207/11013

(57) ABSTRACT

Provided herein are compositions and methods for treating or preventing T cell exhaustion in a subject. In particular, T cells expressing PKC theta K413R mutant or with disrupted CK1G2 activity are provided, which have improved T cell function and tumor control and which are resistant to T cell exhaustion.

### CROSS-REFERENCE TO RELATED APPLICATIONS

This application claims the benefit of and priority to U.S. Provisional Patent Application No. 63/670,716, filed Jul. 12, 2024, the entire contents of which are incorporated herein by reference.

### STATEMENT REGARDING FEDERALLY SPONSORED RESEARCH OR DEVELOPMENT

This invention was made with government support under R01CA216101, T32CA009370, and 5R01AI066232, awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Nov. 4, 2025, is named 138239-0109_SL.xml and is 30,899 bytes in size.

### TECHNICAL FIELD

The present technology generally relates to compositions and methods for treating or preventing T cell exhaustion in a subject in need thereof. In particular, the technology of the present disclosure provides T cells expressing PKC theta K413R mutant or with disrupted CK1G2 activity that are resistant to T cell exhaustion and that have improved T cell function and tumor control properties.

### BACKGROUND

T cell exhaustion is a state of T cell dysfunction that arises during chronic infections and cancer. It is characterized by poor T cell effector function, sustained expression of inhibitory receptors, and a transcriptional state that is distinct from functional effector and memory T cells. Exhaustion negatively affects the capability of the immune system to control infection and tumor growth and metastasis.

After an acute infection, naive antigen-specific CD8⁺ T cells become activated, proliferate, acquire effector functions, and differentiate into effector CD8⁺ T cells. Following clearance of the acute infection, most effector CD8⁺ T cells will undergo apoptosis; however, about 5-10% differentiate into memory CD8⁺ T cells. During chronic infection, severe defects in CD8⁺ T cell responses can develop, and antigen-specific CD8⁺ T cells often fail to differentiate into memory CD8⁺ T cells. Loss of effector function (e.g., T cell exhaustion) occurs in a hierarchical manner, with CD8⁺ T cells progressively losing functions, such as IL-2 production proliferative capacity, and cytotoxicity. Chronic antigen exposure to tumor antigens drives T cell exhaustion (TEX) in cancer and chronic infection.

However, how the kinase cascade downstream of the T cell receptor programs exhaustion is not well understood. Currently, there is an unmet need for compositions and methods that can treat or prevent T cell exhaustion and restore effector function after or during a chronic infection and for the treatment of cancer.

### SUMMARY

In one aspect, the present disclosure provides an isolated nucleic acid molecule comprising a nucleotide sequence encoding a mutant protein kinase C (PKC) theta protein, wherein the nucleotide sequence is operably linked to a heterologous nucleic acid. In some embodiments, the heterologous nucleic acid comprises a promoter. In some embodiments, the promoter comprises an inducible promoter or an overexpression promoter. In some embodiments, the mutant PKC theta protein comprises a K413R mutation. In some embodiments, the isolated nucleotide sequences comprises a nucleotide sequence selected from the group consisting of: (a) a nucleotide sequence set forth in SEQ ID NO: 1; (b) a nucleotide sequence that encodes a polypeptide having the amino acid sequence set forth in SEQ ID NO: 2; (c) a nucleotide sequence that is at least about 85% identical to the nucleotide sequences of (a) or (b), and which encodes a PKC theta protein comprising a K413R mutation. In some embodiments, the encoded mutant PKC theta protein is less susceptible to degradation compared to wild type PKC theta protein. In one aspect, the present disclosure provides a plasmid comprising the isolated nucleic acid molecule of any one of the preceding embodiments. In one aspect, the present disclosure provides a vector comprising the isolated nucleic acid molecule of any one of any one of the preceding embodiments or the plasmid of any one of the preceding embodiments. In some embodiments, the vector comprises a retrovirus, an adeno-associated virus, a lentivirus, a virus-like particle, polymeric nanoparticle, metallic nanoparticles, or a lipid nanoparticle. In one aspect, the present disclosure provides a modified immune cell comprising the isolated nucleic acid molecule of any one of the preceding embodiments or the plasmid of any one of the preceding embodiments, or comprising the isolated nucleic acid of the vector of any one of the preceding embodiments. In some embodiments, the cell produces PKC theta K413R protein. In some embodiments, the modified immune cell is selected from the group consisting of: a T cell; a B cell; a natural killer cell; a dendritic cell; a macrophage; and a monocyte. In some embodiments, the modified immune cell is a T cell. In some embodiments, the T cell is selected from the group consisting of: a naïve T cell; a CD4+ T cell; a CD8+ T cell; a memory T cell; an activated T cell; an exhausted T cell; a tolerant T cell; a chimeric T cell; an antigen-specific T cell; and any combination thereof. In some embodiments, the exhausted T cell is an exhausted CD8⁺ T cell or an exhausted CD4⁺ T cell. In some embodiments, the immune cell is a T cell that produces one or more pro-inflammatory cytokines after a period of stimulation. In some embodiments, the one or more pro-inflammatory cytokines is selected from the group consisting of interleukin 2 (IL-2), interferon gamma (IFN-γ), and tumor necrosis factor (TNF). In some embodiments, the cell further comprises one or more heterologous T cell receptors (TCRs) or chimeric antigen receptors (CARs). In one aspect, the present disclosure provides a composition comprising the modified immune cell of any one of the preceding embodiments and a pharmaceutically acceptable carrier. In one aspect, the present disclosure provides a method of treating cancer in a subject in need thereof, comprising administering to the subject a therapeutically effective amount of one or more of the modified immune cells of any one of the preceding embodiments or the composition of any one of the preceding embodiments. In some embodiments, treatment of the subject results in the subject having one or more of: (i) reduced levels of T cell exhaustion as compared to an untreated control subject with cancer (ii) increased T cell proliferation; (iii) increased T cell viability; or (iv) increased T cell activity. In some embodiments, the treatment reduces the volume of one or more tumors, reduces the mass of one or more tumors, increases IFN-γ production, and/or increases TNF production in the subject. In some embodiments, the one or more modified immune cells is derived from immune cells isolated from the subject. In some embodiments, the immune cells isolated from the subject are obtained from the peripheral blood of the subject. In some embodiments, the immune cells isolated from the subject are obtained from the lymph node, spleen, or tumor of the subject. In some embodiments, the one or more modified immune cells are prepared by contacting the immune cells in vitro with the isolated nucleic acid molecule of any one of the preceding embodiments, the plasmid of any one of the preceding embodiments, or the vector of any one of the preceding embodiments. In some embodiments, the one or more modified immune cells are prepared by contacting a population of peripheral blood mononuclear cells from the subject in vitro with the isolated nucleic acid molecule of any one of the preceding embodiments, the plasmid of any one of the preceding embodiments, or the vector of any one of the preceding embodiments. In some embodiments, the method further comprises expanding the modified immune cells in vitro prior to and/or following contacting the modified immune cells with the isolated nucleic acid molecule of any one of the preceding embodiments, the plasmid of any one of the preceding embodiments, or the vector of any one of the preceding embodiments. In some embodiments, the one or more modified immune cells are administered intravenously, intraperitoneally, subcutaneously, intramuscularly, or intratumorally to the subject. In some embodiments, the method further comprises simultaneously, sequentially, or separately administering an additional therapeutic agent to the subject. In some embodiments, the additional therapeutic agent comprises a chemotherapeutic agent and/or radiation therapy. In some embodiments, the additional therapeutic agent comprises IL-2 therapy, IL-21 therapy, H9T, DR-18, a CAR T cell therapy, an antibody drug conjugate, a JAK inhibitor, IL-18BP, or anti-4-1BB. In some embodiments, the additional therapeutic agent comprises an immune checkpoint blockade inhibitor. In some embodiments, the immune checkpoint blockade inhibitor is selected from the group consisting of: anti-PD-1 inhibitors; anti-PD-L1 inhibitors; anti-IL-10 inhibitors; anti-LAG-3 inhibitors; anti-CTLA-4 inhibitors; anti-Tim3 inhibitors; anti-IL-10R inhibitors; and any combination thereof. In some embodiments, the cancer is selected from the group consisting of: a melanoma; a glioblastoma, a leukemia, and a non-small cell lung cancer. In some embodiments, the subject is a human. In some embodiments, the one or more modified immune cells have an increased capacity to proliferate and/or survive in the subject as compared to one or more unmodified control immune cells. In one aspect, the present disclosure provides a method of treating chronic microbial infection in a subject in need thereof, comprising administering to the subject a therapeutically effective amount of one or more of the modified immune cells of any one of the preceding embodiments or the composition of any one of the preceding embodiments. In some embodiments, treatment of the subject results in the subject having reduced levels of T cell exhaustion as compared to an untreated control subject with a chronic microbial infection. In some embodiments, the treatment of the subject results in the subject having one or more of: (i) reduced levels of T cell exhaustion as compared to an untreated control subject with chronic microbial infection; (ii) increased T cell proliferation; (iii) increased T cell viability; or (iv) increased T cell activity. in the subject. In some embodiments, the one or more modified immune cells is derived from immune cells isolated from the subject. In some embodiments, the immune cells isolated from the subject are obtained from the peripheral blood of the subject. In some embodiments, the one or more modified immune cells are prepared by contacting the immune cells in vitro with the isolated nucleic acid molecule of any one of the preceding embodiments, the plasmid of any one of the preceding embodiments, or the vector of any one of the preceding embodiments. In some embodiments, the one or more modified immune cells are prepared by contacting a population of peripheral blood mononuclear cells from the subject in vitro with the isolated nucleic acid molecule of any one of the preceding embodiments, the plasmid of any one of the preceding embodiments, or the vector of any one of the preceding embodiments. In some embodiments, the method further comprises expanding the modified immune cells in vitro prior to and/or following contacting the modified immune cells with the isolated nucleic acid molecule of any one of the preceding embodiments, the plasmid of any one of the preceding embodiments, or the vector of any one of the preceding embodiments. In some embodiments, the one or more modified immune cells are administered intravenously, intraperitoneally, subcutaneously, intramuscularly, or intratumorally to the subject. In some embodiments, the method further comprises simultaneously, sequentially, or separately administering an additional therapeutic agent to the subject. In some embodiments, the additional therapeutic agent comprises an anti-PD-1 inhibitor, an anti-PD-L1 inhibitor, an anti-IL-10 inhibitor, an anti-LAG-3 inhibitor, an anti-CTLA-4 inhibitor, an anti-TIM3 inhibitor, an anti-IL-10R inhibitor, IL-2, IL-21, H9T, DR-18, a CAR T cell therapy, an antibody drug conjugate, a JAK inhibitor, IL-18BP, anti-4-1BB, and any combination thereof. In some embodiments, the chronic microbial infection is a viral infection, a bacterial infection, a fungal infection, or a protozoan infection. In some embodiments, the chronic microbial infection is caused by a pathogen selected from the group consisting of: Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the subject is a human. In some embodiments, the one or more modified immune cells have an increased capacity to proliferate and/or survive in the subject as compared to one or more unmodified control immune cells.

In another aspect, the present disclosure provides a modified CD8+ T cell comprising a nucleotide sequence set forth in SEQ ID NO: 1.

In a different aspect, the present disclosure provides a modified immune cell comprising a mutant K413R protein kinase C (PKC) theta gene. In some embodiments, the immune cell comprises K413R PKC theta protein. In some embodiments, the modified immune cell is resistant to exhaustion. In some embodiments, the modified immune cell is selected from the group consisting of: a T cell; a B cell; a natural killer cell; a dendritic cell; a macrophage; and a monocyte. In some embodiments, the modified immune cell is a CD8⁺ T cell. In some embodiments, the modified immune cell is a CD4⁺ T cell. In one aspect, the present disclosure provides a composition comprising a therapeutically effective amount of a population of the modified immune cells of any one of the preceding embodiments, and a pharmaceutically acceptable carrier.

In one aspect, the present disclosure provides a method for treating or preventing T cell exhaustion in a subject in need thereof, comprising administering an effective amount of one or more modified immune cells to a subject, wherein the one or more modified immune cells comprises a nucleic acid molecule comprising the nucleotide sequence selected from the group consisting of: (a) a nucleotide sequence set forth in SEQ ID NO: 1; (b) a nucleotide sequence that encodes a polypeptide having the amino acid sequence set forth in SEQ ID NO: 2; and (c) a nucleotide sequence that is at least about 85% identical to the nucleotide sequences of (a) or (b), and which encodes a protein kinase C (PKC) theta protein comprising a K413R mutation. In some embodiments, the subject has cancer. In some embodiments, the cancer is selected from the group consisting of: a melanoma, a glioblastoma, a leukemia, and a non-small cell lung cancer. In some embodiments, the subject is suffering from a microbial infection. In some embodiments, the microbial infection is a viral infection, a bacterial infection, a fungal infection, a protozoan infection, or parasitic infection. In some embodiments, the microbial infection is caused by a pathogen selected from the group consisting of: Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the one or more modified immune cells are derived from immune cells isolated from the subject. In some embodiments, the immune cells isolated from the subject are obtained from the peripheral blood of the subject. In some embodiments, the immune cells isolated from the subject are obtained from the lymph node, spleen, or tumor of the subject. In some embodiments, the one or more modified immune cells are prepared by contacting the immune cells in vitro with the nucleic acid molecule or a plasmid or vector comprising the nucleic acid molecule. In some embodiments, the one or more modified immune cells are prepared by contacting a population of peripheral mononuclear cells from the subject with the nucleic acid molecule or a plasmid or vector comprising the nucleic acid molecule. In some embodiments, the method further comprises expanding the modified immune cells in vitro prior to and/or following contacting the modified immune cells with the nucleic acid molecule or a plasmid or vector comprising the nucleic acid molecule. In some embodiments, the one or more modified immune cells are administered intravenously, intraperitoneally, subcutaneously, intramuscularly, or intratumorally to the subject. In some embodiments, the subject is a human.

In one aspect, the present disclosure provides a kit comprising the isolated nucleic acid molecule of any one of the preceding embodiments, the plasmid of any one of the preceding embodiments, or the vector of any one of the preceding embodiments, and instructions for use thereof to genetically engineer a cell.

In another aspect, the present disclosure provides a method for generating a population of modified immune cells, comprising contacting the immune cells with an agent selected from the group consisting of: (a) a nucleotide sequence set forth in SEQ ID NO: 1; (b) a nucleotide sequence that encodes a polypeptide having the amino acid sequence set forth in SEQ ID NO: 2; (c) a nucleotide sequence that is at least about 85% identical to the nucleotide sequences of (a) or (b), and which encodes a protein kinase C (PKC) theta protein comprising a K413R mutation; (d) a plasmid comprising any one of (a), (b), or (c); and (e) a vector comprising any one of (a), (b), (c), or (d). In some embodiments, the method further comprises expanding the modified immune cells in vitro prior to and/or following contacting the modified immune cells with the agent. In some embodiments, the modified immune cells have an increased proliferative capacity in vivo as compared to unmodified immune cells. In some embodiments, the modified immune cells are resistant to exhaustion.

In one aspect, the present disclosure provides a composition comprising: (a) a mutant protein kinase C (PKC) theta protein, or a nucleic acid sequence encoding the protein; and (b) one or more primary immune cells isolated from a donor subject that has cancer, wherein the one or more primary immune cells are reactive against a cancer-specific antigen. In a different aspect, the present disclosure provides a composition comprising: (a) a mutant protein kinase C (PKC) theta protein, or a nucleic acid sequence encoding the protein; and (b) one or more primary immune cells isolated from a donor subject that has a chronic microbial infection, wherein the one or more primary immune cells are reactive against a microbe-specific antigen. In some embodiments, the cancer is a hematologic cancer or solid tumor. In some embodiments, solid tumor is a carcinoma, adenoma, adenocarcinoma, blastoma, sarcoma, or lymphoma. In some embodiments, the chronic microbial infection is selected from Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the PKC theta protein comprises SEQ ID NO: 2. In some embodiments, the nucleic acid sequence encoding the PKC theta protein comprises SEQ ID NO: 1. In some embodiments, the one or more immune cells comprises one or more lymphocytes. In some embodiments, the one or more lymphocytes comprises a T cell, a B cell, an NK cell, or any combination thereof. In some embodiments, the T cell is selected from the group consisting of naïve T cells, CD4+ T cells, CD8+ T cells, memory T cells, activated T cells, exhausted T cells, tolerant T cells, chimeric B cells, and antigen-specific T cells. In some embodiments, the B cells are selected from the group consisting of naïve B cells, plasma B cells, activated B cells, memory B cells, exhausted B cells, tolerant B cells, chimeric B cells, and antigen-specific B cells. In some embodiments, the one or more lymphocytes is a T-cell receptor modified lymphocyte or a chimeric antigen receptor modified lymphocyte.

In one aspect, the present disclosure provides, a modified immune cell comprising disrupted CK1G2 activity, wherein the modified immune cell has been contacted with an agent that disrupts CK1G2 activity selected from the group consisting of: an antisense oligonucleotide, a shRNA, a siRNA, a zinc finger nuclease, and CRISPR-Cas system. In some embodiments, the modified immune cell produces less biologically active CK1G2 protein as compared to an untreated control cell. In some embodiments, the modified immune cell is selected from the group consisting of: a T cell; a B cell; a natural killer cell; a dendritic cell; a macrophage; and a monocyte. In some embodiments, the modified immune cell is a T cell. In some embodiments, the T cell is selected from the group consisting of: a naïve T cell; a CD4+ T cell; a CD8+ T cell; a memory T cell; an activated T cell; an exhausted T cell; a tolerant T cell; a chimeric T cell; an antigen-specific T cell; and any combination thereof. In some embodiments, the exhausted T cell is an exhausted CD8+ T cell or an exhausted CD4+ T cell. In some embodiments, the immune cell is a T cell that produces one or more pro-inflammatory cytokines after a period of stimulation. In some embodiments, the one or more pro-inflammatory cytokines is selected from the group consisting of interleukin 2 (IL-2), interferon gamma (IFN-γ), and tumor necrosis factor (TNF). In some embodiments, the cell further comprises one or more heterologous T cell receptors (TCRs) or chimeric antigen receptors (CARs). In one aspect, the present disclosure provides a composition comprising the modified immune cell of any one of the preceding embodiments and a pharmaceutically acceptable carrier. In another aspect, the present disclosure provides a method of treating cancer in a subject in need thereof, comprising administering to the subject a therapeutically effective amount of one or more of the modified immune cells of any one of the preceding embodiments or the composition of any one of the preceding embodiments. In some embodiments, treatment of the subject results in the subject having one or more of: (i) reduced levels of T cell exhaustion as compared to an untreated control subject with cancer (ii) increased T cell proliferation; (iii) increased T cell viability; or (iv) increased T cell activity. In some embodiments, the treatment reduces the volume of one or more tumors, reduces the mass of one or more tumors, increases IFN-γ production, and/or increases TNF production in the subject. In some embodiments, the one or more modified immune cells is derived from immune cells isolated from the subject. In some embodiments, the immune cells isolated from the subject are obtained from the peripheral blood of the subject. In some embodiments, the immune cells isolated from the subject are obtained from the lymph node, spleen, or tumor of the subject. In some embodiments, the one or more modified immune cells are prepared by contacting the immune cells in vitro with the agent that disrupts CK1G2 activity. In some embodiments, the one or more modified immune cells are prepared by contacting a population of peripheral blood mononuclear cells from the subject in vitro with the agent that disrupts CK1G2 activity. In some embodiments, the method further comprises expanding the modified immune cells in vitro prior to and/or following contacting the modified immune cells with the agent that disrupts CK1G2 activity. In some embodiments, the one or more modified immune cells are administered intravenously, intraperitoneally, subcutaneously, intramuscularly, or intratumorally to the subject. In some embodiments, the method further comprises simultaneously, sequentially, or separately administering an additional therapeutic agent to the subject. In some embodiments, the additional therapeutic agent comprises a chemotherapeutic agent and/or radiation therapy. In some embodiments, the additional therapeutic agent comprises IL-2 therapy, IL-21 therapy, H9T, DR-18, a CAR T cell therapy, an antibody drug conjugate, a JAK inhibitor, IL-18BP, or anti-4-1BB. In some embodiments, the additional therapeutic agent comprises an immune checkpoint blockade inhibitor. In some embodiments, the immune checkpoint blockade inhibitor is selected from the group consisting of: anti-PD-1 inhibitors; anti-PD-L1 inhibitors; anti-IL-10 inhibitors; anti-LAG-3 inhibitors; anti-CTLA-4 inhibitors; anti-Tim3 inhibitors; anti-IL-10R inhibitors; and any combination thereof. In some embodiments, the cancer is selected from the group consisting of: a melanoma; a glioblastoma, a leukemia, and a non-small cell lung cancer. In some embodiments, the subject is a human. In some embodiments, the one or more modified immune cells have an increased capacity to proliferate and/or survive in the subject as compared to one or more unmodified control immune cells. In another aspect, the present disclosure provides, a method of treating chronic microbial infection in a subject in need thereof, comprising administering to the subject a therapeutically effective amount of one or more of the modified immune cells of any one of the preceding embodiments or the composition of any one of the preceding embodiments. In some embodiments, treatment of the subject results in the subject having reduced levels of T cell exhaustion as compared to an untreated control subject with a chronic microbial infection. In some embodiments, the treatment of the subject results in the subject having one or more of: (i) reduced levels of T cell exhaustion as compared to an untreated control subject with chronic microbial infection; (ii) increased T cell proliferation; (iii) increased T cell viability; or (iv) increased T cell activity. In some embodiments, the one or more modified immune cells is derived from immune cells isolated from the subject. In some embodiments, the immune cells isolated from the subject are obtained from the peripheral blood of the subject. In some embodiments, the one or more modified immune cells are prepared by contacting the immune cells in vitro with the agent that disrupts CK1G2 activity. In some embodiments, the one or more modified immune cells are prepared by contacting a population of peripheral blood mononuclear cells from the subject in vitro with the agent that disrupts CK1G2 activity. In some embodiments, the method further comprises expanding the modified immune cells in vitro prior to and/or following contacting the modified immune cells with the agent that disrupts CK1G2 activity. In some embodiments, the one or more modified immune cells are administered intravenously, intraperitoneally, subcutaneously, intramuscularly, or intratumorally to the subject. In some embodiments, the method further comprises simultaneously, sequentially, or separately administering an additional therapeutic agent to the subject. In some embodiments, the additional therapeutic agent comprises an anti-PD-1 inhibitor, an anti-PD-L1 inhibitor, an anti-IL-10 inhibitor, an anti-LAG-3 inhibitor, an anti-CTLA-4 inhibitor, an anti-TIM3 inhibitor, an anti-IL-10R inhibitor, IL-2, IL-21, H9T, DR-18, a CAR T cell therapy, an antibody drug conjugate, a JAK inhibitor, IL-18BP, anti-4-1BB, and any combination thereof. In some embodiments, the chronic microbial infection is a viral infection, a bacterial infection, a fungal infection, or a protozoan infection. In some embodiments, the chronic microbial infection is caused by a pathogen selected from the group consisting of: Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the subject is a human. In some embodiments, the one or more modified immune cells have an increased capacity to proliferate and/or survive in the subject as compared to one or more unmodified control immune cells.

In one aspect, the present disclosure provides a modified immune cell comprising disrupted CK1G2 activity, wherein the modified immune cell was contacted with an agent that disrupts CK1G2 activity selected from the group consisting of: an antisense oligonucleotide, a shRNA, a siRNA, a zinc finger nuclease, and CRISPR-Cas system. In some embodiments, the modified immune cell produces less biologically active CK1G2 protein as compared to an unmodified control cell. In some embodiments, the modified immune cell does not produce full length CK1G2 protein. In some embodiments, the modified immune cell is resistant to exhaustion. In some embodiments, the modified immune cell is selected from the group consisting of: a T cell; a B cell; a natural killer cell; a dendritic cell; a macrophage; and a monocyte. In some embodiments, the modified immune cell is a CD8⁺ T cell. In some embodiments, the modified immune cell is a CD4⁺ T cell. In another aspect, the present disclosure provides a composition comprising a therapeutically effective amount of a population of the modified immune cells of any one of the preceding embodiments, and a pharmaceutically acceptable carrier.

In a different aspect, the present disclosure provides a method for treating or preventing T cell exhaustion in a subject in need thereof, comprising administering an effective amount of one or more modified immune cells to a subject, wherein the one or more modified immune cells were contacted with an agent that disrupts CK1G2 activity selected from the group consisting of: an antisense oligonucleotide, a shRNA, a siRNA, a zinc finger nuclease, and a CRISPR-Cas system. In some embodiments, the subject has cancer. In some embodiments, the cancer is selected from the group consisting of: a melanoma, a glioblastoma, a leukemia, and a non-small cell lung cancer. In some embodiments, the subject is suffering from a microbial infection. In some embodiments, the microbial infection is a viral infection, a bacterial infection, a fungal infection, a protozoan infection, or parasitic infection. In some embodiments, the microbial infection is caused by a pathogen selected from the group consisting of: Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the one or more modified immune cells are derived from immune cells isolated from the subject. In some embodiments, the immune cells isolated from the subject are obtained from the peripheral blood of the subject. In some embodiments, the immune cells isolated from the subject are obtained from the lymph node, spleen, or tumor of the subject. In some embodiments, the one or more modified immune cells are prepared by contacting the immune cells in vitro with the agent that disrupts CK1G2 activity. In some embodiments, the one or more modified immune cells are prepared by contacting a population of peripheral mononuclear cells from the subject with the agent that disrupts CK1G2 activity. In some embodiments, the method further comprises expanding the modified immune cells in vitro prior to and/or following contacting the modified immune cells with the agent that disrupts CK1G2 activity. In some embodiments, the one or more modified immune cells are administered intravenously, intraperitoneally, subcutaneously, intramuscularly, or intratumorally to the subject. In some embodiments, the subject is a human.

In one aspect, the present disclosure provides a kit comprising an agent that disrupts CK1G2 activity, or a nucleic acid encoding the same, wherein the agent is selected from the group consisting of: an antisense oligonucleotide, a shRNA, a siRNA, a zinc finger nuclease, and CRISPR-Cas system, and instructions for use thereof to genetically engineer a cell.

In yet another aspect, the present disclosure provides a method for generating a population of modified immune cells, comprising contacting the immune cells with an agent that disrupts CK1G2 activity selected from the group consisting of: (a) an anti-sense oligonucleotide; (b) a shRNA; (c) a siRNA; (d) a zinc finger nuclease; and (e) a CRISPR-Cas system. In some embodiments, the method further comprises expanding the modified immune cells in vitro prior to and/or following contacting the modified immune cells with the agent. In some embodiments, the modified immune cells have an increased proliferative capacity in vivo as compared to unmodified immune cells. In some embodiments, the modified immune cells are resistant to exhaustion.

In one aspect, the present disclosure provides a composition comprising: (a) an agent that disrupts CK1G2 activity selected from the group consisting of an antisense oligonucleotide, a shRNA, a siRNA, a zinc finger nuclease, and CRISPR-Cas system; and (b) one or more primary immune cells isolated from a donor subject that has cancer, wherein the one or more primary immune cells are reactive against a cancer-specific antigen. In a different aspect, the present disclosure provides a composition comprising: (a) an agent that disrupts CK1G2 activity selected from the group consisting of an antisense oligonucleotide, a shRNA, a siRNA, a zinc finger nuclease, and CRISPR-Cas system; and (b) one or more primary immune cells isolated from a donor subject that has a chronic microbial infection, wherein the one or more primary immune cells are reactive against a microbe-specific antigen. In some embodiments, the cancer is a hematologic cancer or solid tumor. In some embodiments, the solid tumor is a carcinoma, adenoma, adenocarcinoma, blastoma, sarcoma, or lymphoma. In some embodiments, the chronic microbial infection is selected from Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the agent that disrupts CK1G2 activity is comprised within a vector selected from the group consisting of: an adeno-associated virus, a virus-like particle, and a lipid nanoparticle. In some embodiments, the agent that disrupts CK1G2 activity is a CRISPR-Cas system. In some embodiments, the one or more immune cells comprises one or more lymphocytes. In some embodiments, the one or more lymphocytes comprises a T cell, a B cell, an NK cell, or any combination thereof. In some embodiments, the T cell is selected from the group consisting of naïve T cells, CD4+ T cells, CD8+ T cells, memory T cells, activated T cells, exhausted T cells, tolerant T cells, chimeric B cells, and antigen-specific T cells. In some embodiments, the B cells are selected from the group consisting of naïve B cells, plasma B cells, activated B cells, memory B cells, exhausted B cells, tolerant B cells, chimeric B cells, and antigen-specific B cells. In some embodiments, the one or more lymphocytes is a T-cell receptor modified lymphocyte or a chimeric antigen receptor modified lymphocyte.

In another aspect, the present disclosure provides an isolated nucleic acid molecule comprising a nucleotide sequence encoding a wild type protein kinase C (PKC) theta protein, wherein the nucleotide sequence is operably linked to an overexpression promoter. In some embodiments, the isolated nucleic acid sequence comprises a nucleotide sequence selected from the group consisting of: (a) a nucleotide sequence set forth in SEQ ID NO: 3; (b) a nucleotide sequence that encodes a polypeptide having the amino acid sequence set forth in SEQ ID NO: 4; (c) a nucleotide sequence that is at least about 85% identical to the nucleotide sequences of (a) or (b). In one aspect, the present disclosure provides a plasmid comprising the isolated nucleic acid molecule of any one of the preceding embodiments. In a different aspect the present disclosure provides a vector comprising the isolated nucleic acid molecule of any one of the preceding embodiments or the plasmid of any one of the preceding embodiments. In some embodiments, the vector comprises a retrovirus, an adeno-associated virus, a lentivirus, a virus-like particle, polymeric nanoparticle, metallic nanoparticles, or a lipid nanoparticle. In some embodiments, the isolated nucleic acid molecule of any one of the preceding embodiments or the plasmid of any one of the preceding embodiments, or comprising the isolated nucleic acid of the vector of any one of the preceding embodiments. In some embodiments, the modified immune cell is selected from the group consisting of: a T cell; a B cell; a natural killer cell; a dendritic cell; a macrophage; and a monocyte. In some embodiments, the modified immune cell is a T cell. In some embodiments, the T cell is selected from the group consisting of: a naïve T cell; a CD4+ T cell; a CD8+ T cell; a memory T cell; an activated T cell; an exhausted T cell; a tolerant T cell; a chimeric T cell; an antigen-specific T cell; and any combination thereof. In some embodiments, the exhausted T cell is an exhausted CD8⁺ T cell or an exhausted CD4⁺ T cell. In some embodiments, the immune cell is a T cell that produces one or more pro-inflammatory cytokines after a period of stimulation. In some embodiments, the one or more pro-inflammatory cytokines is selected from the group consisting of interleukin 2 (IL-2), interferon gamma (IFN-γ), and tumor necrosis factor (TNF). In some embodiments, the cell further comprises one or more heterologous T cell receptors (TCRs) or chimeric antigen receptors (CARs). In one aspect, the present disclosure provides a composition comprising the modified immune cell of any one of the preceding embodiments and a pharmaceutically acceptable carrier. In a different aspect, the present disclosure provides a method of treating cancer in a subject in need thereof, comprising administering to the subject a therapeutically effective amount of one or more of the modified immune cells of any one the preceding embodiments or the composition of any one the preceding embodiments. In some embodiments, treatment of the subject results in the subject having one or more of: (i) reduced levels of T cell exhaustion as compared to an untreated control subject with cancer (ii) increased T cell proliferation; (iii) increased T cell viability; or (iv) increased T cell activity. In some embodiments, the treatment reduces the volume of one or more tumors, reduces the mass of one or more tumors, increases IFN-7 production, and/or increases TNF production in the subject. In some embodiments, the one or more modified immune cells is derived from immune cells isolated from the subject. In some embodiments, the immune cells isolated from the subject are obtained from the peripheral blood of the subject. In some embodiments, the immune cells isolated from the subject are obtained from the lymph node, spleen, or tumor of the subject. In some embodiments, the one or more modified immune cells are prepared by contacting the immune cells in vitro with the isolated nucleic acid molecule of any one the preceding embodiments, the plasmid of any one the preceding embodiments, or the vector of any one the preceding embodiments. In some embodiments, the one or more modified immune cells are prepared by contacting a population of peripheral blood mononuclear cells from the subject in vitro with the isolated nucleic acid molecule of any one of any one the preceding embodiments, the plasmid of any one the preceding embodiments, or the vector of any one the preceding embodiments. In some embodiments, the method further comprises expanding the modified immune cells in vitro prior to and/or following contacting the modified immune cells with the isolated nucleic acid molecule of any one the preceding embodiments, the plasmid of any one the preceding embodiments, or the vector of any one the preceding embodiments. In some embodiments, the one or more modified immune cells are administered intravenously, intraperitoneally, subcutaneously, intramuscularly, or intratumorally to the subject. In some embodiments, the method further comprises simultaneously, sequentially, or separately administering an additional therapeutic agent to the subject. In some embodiments, the additional therapeutic agent comprises a chemotherapeutic agent and/or radiation therapy. In some embodiments, the additional therapeutic agent comprises IL-2 therapy, IL-21 therapy, H9T, DR-18, a CAR T cell therapy, an antibody drug conjugate, a JAK inhibitor, IL-18BP, or anti-4-1BB. In some embodiments, the additional therapeutic agent comprises an immune checkpoint blockade inhibitor. In some embodiments, the immune checkpoint blockade inhibitor is selected from the group consisting of: anti-PD-1 inhibitors; anti-PD-L1 inhibitors; anti-IL-10 inhibitors; anti-LAG-3 inhibitors; anti-CTLA-4 inhibitors; anti-Tim3 inhibitors; anti-IL-10R inhibitors; and any combination thereof. In some embodiments, the cancer is selected from the group consisting of: a melanoma; a glioblastoma, a leukemia, and a non-small cell lung cancer. In some embodiments, the subject is a human. In some embodiments, the one or more modified immune cells have an increased capacity to proliferate and/or survive in the subject as compared to one or more unmodified control immune cells. In a different aspect, the present disclosure provides a method of treating chronic microbial infection in a subject in need thereof, comprising administering to the subject a therapeutically effective amount of one or more of the modified immune cells of any one the preceding embodiments or the composition of any one the preceding embodiments. In some embodiments, treatment of the subject results in the subject having reduced levels of T cell exhaustion as compared to an untreated control subject with a chronic microbial infection. In some embodiments, the treatment of the subject results in the subject having one or more of: (i) reduced levels of T cell exhaustion as compared to an untreated control subject with chronic microbial infection; (ii) increased T cell proliferation; (iii) increased T cell viability; and (iv) increased T cell activity. in the subject. In some embodiments, the one or more modified immune cells is derived from immune cells isolated from the subject. In some embodiments, the immune cells isolated from the subject are obtained from the peripheral blood of the subject. In some embodiments, the one or more modified immune cells are prepared by contacting the immune cells in vitro with the isolated nucleic acid molecule of any one the preceding embodiments, the plasmid of any one the preceding embodiments, or the vector of any one the preceding embodiments. In some embodiments, the one or more modified immune cells are prepared by contacting a population of peripheral blood mononuclear cells from the subject in vitro with the isolated nucleic acid molecule of any one the preceding embodiments, the plasmid of any one the preceding embodiments, or the vector of any one the preceding embodiments. In some embodiments, the method further comprises expanding the modified immune cells in vitro prior to and/or following contacting the modified immune cells with the isolated nucleic acid molecule of any one the preceding embodiments, the plasmid of any one the preceding embodiments, or the vector of any one the preceding embodiments. In some embodiments, the one or more modified immune cells are administered intravenously, intraperitoneally, subcutaneously, intramuscularly, or intratumorally to the subject. In some embodiments, the method further comprises simultaneously, sequentially, or separately administering an additional therapeutic agent to the subject. In some embodiments, the additional therapeutic agent comprises an anti-PD-1 inhibitor, an anti-PD-L1 inhibitor, an anti-IL-10 inhibitor, an anti-LAG-3 inhibitor, an anti-CTLA-4 inhibitor, an anti-TIM3 inhibitor, an anti-IL-10R inhibitor, IL-2, IL-21, H9T, DR-18, a CAR T cell therapy, an antibody drug conjugate, a JAK inhibitor, IL-18BP, anti-4-1BB, and any combination thereof. In some embodiments, the chronic microbial infection is a viral infection, a bacterial infection, a fungal infection, or a protozoan infection. In some embodiments, the chronic microbial infection is caused by a pathogen selected from the group consisting of: Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the subject is a human. In some embodiments, the one or more modified immune cells have an increased capacity to proliferate and/or survive in the subject as compared to one or more unmodified control immune cells.

### BRIEF DESCRIPTION OF THE DRAWINGS

FIGS. 1A-1B show that differences in kinase signaling of T_{PROG} and T_{TERM} cells correlate with a change in the expression of PKC proteins. FIG. 1A shows mean fluorescence intensities (MFIs) from phospho-flow cytometry of the indicated markers in T_{PROG} or T_{TERM} cells from LCMV Cl 13 infected mice were stimulated with PMA ex vivo for 30 mins (all but c-Fos) or 4 hours (c-Fos). FIG. 1B shows the expression of PKC theta and eta by T cell subset from LCMV Cl 13.

FIGS. 1C-1K show that PKC theta is required for progenitor exhausted T cells (T_{PROG}) while PKC eta promotes terminal exhaustion (T_{TERM}) differentiation of T cells. FIG. 1C is a western blot validating PKC theta and eta P14 T cell knockouts (left panel), and a validation of PKC knockout by flow cytometry (right panel). FIG. 1D is a diagram showing an experimental timeline wherein mice were infected with LCMV clone 13, and congenic P14 T cells were negative selected and activated in vitro via platebound αCD3/28. After 24 hours, cells were subjected to Cas9-sgRNA electroporation to delete PKC theta or PKC eta and subsequently transferred into infected mice. FIGS. 1E-1G show the frequencies of transferred cells as a fraction of all live CD8 T cells (FIG. 1E), and frequencies of T_{PROG} or KLRG1+ cells of transferred PD1+ CD8 T cells (FIGS. 1F-1G), at 8 days post infection. FIG. 1H shows the transferred cell populations, which have different genotypes, as fractions of the total CD8 population. FIGS. 1I-1J show markers as fractions of the PD1+ transferred cell population and TOX MFI (FIG. 1K) in transferred PD1+ cells, at 28 days post infection.

FIGS. 2A-2E show that chronic agonism of PKC is sufficient to drive a terminal exhaustion signature in CD8⁺ T cells. FIG. 2A shows a schematic of in vitro exhaustion experiments. FIG. 2B shows representative flow plots showing the effect of chronic PMA treatment on T cells in vitro. FIG. 2C shows cumulative bar graphs of PMA effects on in vitro exhausted cells. FIG. 2D shows the effects of KO of Prkcq or Prkch on responsiveness to PMA during in vitro exhaustion. FIG. 2E shows nCounter data highlighting PMA-driven gene expression changes. T_{TERM}-associated genes are in red, while T_{PROG}-associated genes are in blue. Data are shown as log 2 fold change.

FIGS. 3A-3D show that phospho-proteomics reveal different kinase targets for PKC theta and eta. FIG. 3A shows a schematic of the preparation of T cells for global phospho-proteomics. FIG. 3B is a cartoon highlighting and simplifying major known pathways downstream of PKC. FIG. 3C shows PKC theta vs eta targets, downstream of anti-CD3 restimulation. FIG. 3D shows PKC theta targets, unstimulated vs anti-CD3 restimulated.

FIGS. 4A-4D show that PKC eta activates downstream kinases, including activation of casein kinase I G2. FIG. 4A is a schematic of in vitro exhaustion experiments used to screen inhibitors of kinases downstream of PKC theta and eta. FIG. 4B shows the screening effects of inhibitors of kinase families putatively downstream of the PKCs for IFNγ production (left), or in a secondary screen, for markers of T_{PROG} differentiation (right). FIG. 4C shows a Western blot analysis of p38 after TCR or PMA stim in PKC KO genotypes. FIG. 4D shows a graph of phospho-flow cytometric analyses of several phospho-epitopes (p38, S6, INK, ERK1/2, NF-κB p65, and ATF2/7) responding to PMA stimulation in vitro, plotted according to genotype and stimulation condition.

FIGS. 5A-5I show that agonism of PKCs drive PKC theta degradation through ubiquitylation of K413 and that a PKC theta K413R mutant variant is degradation resistant and enhances T cell activation while reducing exhaustion. FIGS. 5A and 5B are Western blots showing protein degradation kinetics of PKC theta and eta during constitutive activation by PMA. FIG. 5C is a cartoon schematic of the regulation of PKC kinase activity. FIG. 5D is a Western blot showing KO of Peli1, the E3 ligase for PKC theta, (top) and its effect on PKC theta protein levels by subset in P14 T cells responding to LCMV Clone 13 at 28 days post infection (bottom). FIG. 5E shows the domain architecture of PKC theta. Numbers represent the amino acids defining each domain and the three phosphorylation sites key for PKC kinase activity. FIG. 5F shows the results of a test of whether PKC theta's kinase domain can be targeted for degradation in the absence of its N-terminal domains. An empty vector control or truncated PKC theta variants (with murine Prkcq amino acid cutoffs indicated on the graph) were overexpressed in T cells, and PKC theta levels were measured after a 24 hours of PMA treatment. FIG. 5G shows the results of a two-round K-to-R mutagenesis screen to identify lysine residues required for PKC activity-induced degradation after 24 hours of PMA treatment. Lysine residues were first mutated in blocks of four to six at a time (left), and then individual lysine residues were mutated within the 409-451 subset (right). FIGS. 5H-5I are graphs showing the cytokine levels for P14 T cells that were transduced with EV or PKC OE vectors, subjected to in vitro exhaustion, and assessed for their capacity to produce cytokines.

FIGS. 6A-6O show that overexpression of non-degradable PKC theta K413R in T cells promotes anti-tumor functions in vivo. FIG. 6A is a diagram showing a timeline of adoptive transfer experiments in LCMV Cl 13. FIG. 6B-C are graphs showing the MFI of PKC theta among all transferred GFP+ (FIG. 6B) and GFP+ T_{TERM} cells (FIG. 6C). FIGS. 6D-6F are graphs showing the numbers of total GFP+ cells of the indicated genotypes, in total (FIG. 6D), or in SLAMF6+ TIM3− (FIG. 6E) or SLAMF6− TIM3+ (FIG. 6F) subsets. FIGS. 6G-6I show the normalized MFI of the indicated markers in LCMV Cl 13. FIG. 6J is a diagram showing a timeline of adoptive transfer experiments in B16gp33 tumor-bearing mice. FIG. 6K is a graph of endpoint tumor masses for mice receiving an elevated dose of 4×10⁵ adoptively transferred P14 T cells of the indicated genotypes. FIGS. 6L-6M are graphs showing the tumor volumes (FIG. 6L) and masses at endpoint (FIG. 6M) of tumors for mice receiving 2×10⁵ P14 T cells of the indicated genotypes. FIGS. 6N-6O are graphs showing cytokine producing functionality of P14 T cells infiltrating B16gp33 tumors by genotype.

FIGS. 7A-7P show that deletion of the putative PKC eta target CK1G2 reduces T cell exhaustion. FIG. 7A is a diagram showing a timeline of casein kinase I gene deletions in LCMV Cl 13 adoptive transfer experiments. FIG. 7B is a graph showing the frequency of the indicated genotype in transferred P14 T cells among all CD8 T cells. FIGS. 7C-7F are graphs showing the frequency of the indicated subsets among P14 T cells in LCMV Cl 13 infected mice at 8 days post infection. FIG. 7G is a is a graph showing the cytokine producing functionality in PD1+P14 T cells in LCMV Cl 13 infected mice at 8 days post infection. FIG. 7H-I are graphs showing mean fluorescence intensity of the indicated markers at 8 days post infection. FIG. 7J is a graph showing the frequency of transferred cell genotypes among all live CD8 T cells at 28 days post-infection. FIG. 7K is a graph showing the cytokine producing functionality in PD1+P14 T cells in LCMV Cl 13 infected mice at 28 days post infection. FIG. 7L is a graph showing the frequency of cytokine producing cells among terminally exhausted T_{TERM} P14 T cells in LCMV Cl 13 infected mice at 28 days post infection. FIG. 7M is a diagram showing a timeline of casein kinase I G2 gene deletion in P14 T cells transferred into B16-gp33 tumor bearing mice. FIG. 7N is a graph showing the tumor growth curves of B16-gp33 tumor bearing mice that received P14 T cells deleted for Csnk1g2 or a non-targeting negative control guide RNA. FIG. 7O is a graph showing the tumor masses of mice receiving P14 T cells deleted for Csnk1g2 or a non-targeting control guide. FIG. 7P is a graph showing PD-1 expression in T_{TERM} P14 T cells deleted for Csnk1g2 or a non-targeting control, infiltrating B16-gp33 tumors.

FIG. 8 is a graph showing that deletion of PRKCH or CSNK1G2 in human GD2 CD8 CAR-T cells leads to an improved anti-tumor response against human U87 tumors implanted in NSG mice. Growth curves showing the volume in mm³ of U87 tumors implanted on the side of immunodeficient NSG mice. Tumors were measured every other day by calipers. Growth curves show the mean volume+/−s.e.m. Significance is shown as ****P<0.0001 as determined by 2-way ANOVA.

### DETAILED DESCRIPTION

It is to be appreciated that certain aspects, modes, embodiments, variations, and features of the present technology are described below in various levels of detail in order to provide a substantial understanding of the present technology.

### I. Definitions

The definitions of certain terms as used in this specification are provided below. Unless defined otherwise, all technical and scientific terms used herein generally have the same meaning as commonly understood by one of ordinary skill in the art to which this present technology belongs.

As used herein, the singular forms “a,” “an,” and “the” are intended to include the plural forms as well, unless the context clearly indicates otherwise.

As used herein, the term “about” means that a value can vary +/−20%, +/−15%, +/−10% or +/−5% and remain within the scope of the present disclosure. For example, “a concentration of about 200 IU/mL” encompasses a concentration between 160 IU/mL and 240 IU/mL.

As used herein, the term “administration” of an agent to a subject includes any route of introducing or delivering the agent to a subject to perform its intended function. Administration can be carried out by any suitable route, including intravenously, intramuscularly, intraperitoneally, or subcutaneously. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term CK1G2 refers to the Csnk1g2 gene or the polypeptide encoded thereby. In some embodiments, the accession number of the CK1G2 gene is NCBI Reference Sequence: NM_001319.7. In some embodiments, the CK1G2 gene comprises the sequence of SEQ ID NO. 9.

As used herein the term “disruption” refers to a disrupted gene with decreased levels of expression of a gene product (e.g., protein or RNA) as compared to a wild-type levels. Disruption may include decreased expression of a gene product via a therapeutic nucleic acid or oligonucleotide agent, such as an antisense oligonucleotide (ASO), RNA interference (RNAi), including anti-sense, sense co-suppression, microRNA (miRNA), short interfering RNA (siRNA), short hairpin RNA (shRNA), a gRNA, or an sgRNA. In some embodiments, disruption of a gene results in decreased translation of an RNA gene product. Disruptions may include mutations, including insertions, deletions, substitutions (e.g., transitions, transversion), transpositions, inversions, knockouts, and combinations thereof. Mutations may involve only a single nucleotide (e.g., a point mutation or a single nucleotide polymorphism) or multiple nucleotides. In some embodiments, the mutation causes a phenotypic change, for example, the expression level of the encoded product is altered, or the encoded product itself is altered. In some embodiments, disruption results in a decrease in the amount of biologically active encoded product. In other embodiments, a disruption or mutation may result in an expressed protein with activity that is lower as compared to the activity of the expressed protein from the wild-type. In some embodiments, disruption is achieved with a CRISPR/Cas system, a meganuclease, a TALEN, a ZFN, or any combination thereof. As used herein, in some embodiments, a mutant gene may comprise, but is not limited to, a deletion of all or a portion of the gene; deletion of a regulatory element that controls gene expression, a frameshift mutation of the gene, or replacement of all or a portion of a gene.

As used herein, the term “effective amount” or “therapeutically effective amount” refers to a quantity of an agent sufficient to achieve a desired therapeutic effect. In the context of therapeutic applications, the amount of a therapeutic protein administered to the subject can depend on the type and severity of the infection and on the characteristics of the individual, such as general health, age, sex, body weight and tolerance to drugs. It can also depend on the degree, severity and type of disease. The skilled artisan will be able to determine appropriate dosages depending on these and other factors.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

As used herein the term immune cell refers to any cell that plays a role in the immune response. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes.

The term “lymphocyte” refers to all immature, mature, undifferentiated and differentiated white lymphocyte populations including tissue specific and specialized varieties. It encompasses, by way of non-limiting example, B cells, T cells, NKT cells, and NK cells. In some embodiments, lymphocytes includes all B cell lineages including pre-B cells, progenitor B cells, early pro-B cells, late pro-B cells, large pre-B cells, small pre-B cells, immature B cells, mature B cells, plasma B cells, memory B cells, B-1 cells, and B-2 cell populations. In some embodiments, lymphocytes includes all T cell lineages including double negative thymocytes, double positive thymocytes, single positive thymocytes, naïve T cells, CD4+ T cells, CD8+ T cells, memory T cells, activated T cells, exhausted T cells, tolerant T cells, chimeric T cells, and antigen-specific T cells.

As used herein, the term T cell includes naïve T cells, CD4+ T cells, CD8+ T cells, memory T cells, activated T cells, exhausted T cells, tolerant T cells, chimeric T cells, and antigen-specific T cells.

The term “B cell” or “B cells” refers to, by way of non-limiting example, a pre-B cell, progenitor B cell, early pro-B cell, late pro-B cell, large pre-B cell, small pre-B cell, immature B cell, mature B cell, naïve B cells, plasma B cells, activated B cells, exhausted B cells, tolerant B cells, chimeric B cells, antigen-specific B cells, memory B cell, B-1 cell, and B-2 cell populations.

As used herein “adoptive cell therapeutic composition” refers to any composition comprising cells suitable for adoptive cell transfer. In exemplary, non-limiting embodiments, the adoptive cell therapeutic composition comprises a cell type selected from a group consisting of a tumor infiltrating lymphocyte (TIL), TCR (i.e., heterologous T cell receptor) modified lymphocytes and CAR (i.e., chimeric antigen receptor) modified lymphocytes. In some embodiments, the adoptive cell therapeutic composition comprises a cell type selected from a group consisting of T cells, exhausted T cells, CD8+ cells, CD4+ cells, NK-cells, delta-gamma T cells, and regulatory T cells. In some embodiments, TILs, T cells, CD8+ cells, CD4+ cells, NK cells, delta-gamma T cells, regulatory T cells, or peripheral blood mononuclear cells form the adoptive cell therapeutic composition. In some embodiments, the adoptive cell therapeutic composition comprises T cells. In some embodiments, the adoptive cell therapeutic composition may be a composition comprising one or more primary immune cells isolated from a donor subject which comprise disrupted CK1G2 activity or a nucleic acid sequence encoding a PKC theta K413R mutant.

As used herein, the term “exhausted immune cell,” “exhausted T cell,” and “exhausted B cell” refer to dysfunctional T cells and B cells. Exhausted immune cells, exhausted T cells, and exhausted B cells are characterized by progressive loss of effector functions during chronic infections or cancer with some functions that are exhausted early (e.g., IL-2, cytotoxicity, and proliferation), whereas others (e.g., IFN-7) persist longer.

The terms “PKC theta,” “PKCθ,” and “Prkcq” gene are synonyms. They refer to a nucleic acid sequence that encodes a PKC theta (“PKCθ” or “Prkcq”) polypeptide. In some embodiments, the Prkcq gene is a mutant variant of the wild type Prkcq gene. In some embodiments, the Prkcq gene comprises a nucleotide sequence of at least 2000 nucleotides that is at least 60% to 100% identical or homologous, e.g., at least 60, 65%, 70%, 75%, 80%, 85%, 86%, 87%, 88%, 90%, 91%, 92%, 94%, 95%, 96%, 97%, 98%, or any other percent from about 70% to about 100% identical to SEQ ID NO: 1. In some embodiments, the PKC theta gene encodes a kinase.

The terms “PKC theta protein,” “PKC theta polypeptide,” and “PKC theta sequence” are synonyms. In some embodiments, the PKC theta protein is a mutant variant of the wild type PKC theta protein. In some embodiments, the PKC theta protein comprises an amino acid sequence of at least 650 amino acids that is at least 60% to 100% identical or homologous, e.g., at least 60, 65%, 70%, 75%, 80%, 85%, 86%, 87%, 88%, 89%, 90%, 91%, 92%, 93%, 94%, 95%, 96%, 97%, 98%, or any other percent from about 70% to about 100% identical to SEQ ID NO: 2. The sequence of SEQ ID NO: 2 is:

(SEQ ID NO: 2)
MSPFLRIGLSNFDCGSCQSCQGEAVNPYCAVLVKEYVESENGQMYI
QKKPTMYPPWDSTFDAHINKGRVMQIIVKGKNVDLISETTVELYS
LAERCRKNNGKTEIWLELKPQGRMLMNARYFLEMSDTKDMNEFET
EGFFALHQRRGAIKQAKVHHVKCHEFTATFFPQPTFCSVCHEFVW
GLNKQGYQCRQCNAAIHKKCIDKVIAKCTGSAINSRETMFHKERF
KIDMPHRFKVYNYKSPTFCEHCGTLLWGLARQGLKCDACGMNVHH
RCQTKVANLCGINQKLMAEALAMIESTQQARCLRDTEQIFREGPV
EIGLPCSIKNEARPPCLPTPGKREPQGISWESPLDEVDKMCHLPE
PELNKERPSLQIKLKIEDFILHKMLGKGSFGKVFLAEFKKTNQFF
AIKALKRDVVLMDDDVECTMVEKRVLSLAWEHPFLTHMFCTFQTK
ENLFFVMEYLNGGDLMYHIQSCHKFDLSRATFYAAEIILGLQFLH
SKGIVYRDLKLDNILLDKDGHIKIADFGMCKENMLGDAKTNTFCG
TPDYIAPEILLGQKYNHSVDWWSFGVLLYEMLIGQSPFHGQDEEE
LFHSIRMDNPFYPRWLEKEAKDLLVKLFVREPEKRLGVRGDIRQH
PLFREINWEELERKEIDPPFRPKVKSPFDCSNFDKEFLNEKPRLS
FADRALINSMDQNMFRNFSFMNPGMERLIS*

In some embodiments, the PKC theta polypeptide is a complete PKC theta polypeptide sequence. In some embodiments, the PKC theta polypeptide is a partial PKC theta polypeptide sequence. In some embodiments, the PKC theta polypeptide comprises at least 650 consecutive amino acids of SEQ ID NO: 2. In some embodiments, the PKC theta polypeptide comprises at least 650 consecutive amino acids of SEQ ID NO: 2 and retains at least one PKC theta activity. In some embodiments, the PKC theta polypeptide comprises at least 650, at least 660, at least 670, at least 680, at least 690 or at least 700 consecutive amino acids of SEQ ID NO: 2. In some embodiments, the PKC theta polypeptide comprises at least 650, at least 660, at least 670, at least 680, at least 690, or at least 700 consecutive amino acids of SEQ ID NO: 2 and retains at least one PKC theta activity.

In some embodiments, a PKC theta polypeptide comprises an amino acid sequence that is at least 40% to 100% identical, e.g., at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or any other percent from about 40% to about 100% identical to SEQ ID NO: 2. In some embodiments, PKC theta polypeptide refers to a polymer of 706 amino acids, a PKC theta polypeptide that has not undergone any post-translational modifications. In some embodiments, PKC theta polypeptide refers to a polymer of 706 amino acids that has undergone post-translational modifications. In some embodiments, the PKC theta polypeptide is a kinase. In some embodiments, the PKC theta polypeptide promotes one or more of T cell activation, T cell proliferation, T cell viability, and/or reduces or prevents T cell exhaustion. In some embodiments, a PKC theta polypeptide includes one or more of those described above, and includes one or more post-translational modifications (e.g., acetylation). In some embodiments, the PKC theta polypeptides comprise one or more additional amino acid residues at the N-terminus or C-terminus of the polypeptide.

Proteins suitable for use in the methods described herein also includes functional variants, including proteins having between 1 to 15 amino acid changes, e.g., 1, 2, 3, 4, 5, 6, 7, 8, 9, 10, 11, 12, 13, 14, or 15 amino acid substitutions, deletions, or additions, compared to the amino acid sequence of any protein described herein. In other embodiments, the altered amino acid sequence is at least 75% identical, e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any protein inhibitor described herein. Such sequence-variant proteins are suitable for the methods described herein as long as the altered amino acid sequence retains sufficient biological activity to be functional in the compositions and methods described herein. Where amino acid substitutions are made, the substitutions can be conservative amino acid substitutions. Among the common, naturally occurring amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et al. (1992), Proc. Natl Acad. Sci. USA, 89:10915-10919). Accordingly, the BLOSUM62 substitution frequencies are used to define conservative amino acid substitutions that, in some embodiments, are introduced into the amino acid sequences described or disclosed herein. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

The term “PKC theta activity” or “PKC theta biological activity” or “biologically active PKC theta” or “biological activity of PKC theta” includes one or more of T cell activation, T cell proliferation, T cell viability, and/or reduces or prevents T cell exhaustion. By way of example and not by way of limitation, PKC theta activity includes expansion and maintenance of T cell populations, increased T cell activity (e.g., inflammatory cytokine production), and reduction of T cell exhaustion.

The terms “patient,” “subject,” “individual,” and the like are used interchangeably herein, and refer to an animal, typically a mammal. In one embodiment, the patient, subject, or individual is a mammal. In one embodiment, the patient, subject or individual is a human. In some embodiments the patient, subject or individual is an animal, such as, but not limited to, domesticated animals, such as equine, bovine, murine, ovine, canine, and feline.

“Promoter” connotes a region of DNA upstream from the start of transcription that is involved in recognition and binding of RNA polymerase and other proteins to initiate transcription. A “constitutive promoter” is one that is active throughout the life of the cell and under most conditions. Tissue-specific, tissue-preferred, cell type-specific, and inducible promoters constitute the class of “non-constitutive promoters.” Tissue-specific regulatory elements are known in the art. In some embodiments, the tissue-specific promoter is immune cell specific. “Operably linked” or “operatively linked” refers to a functional linkage between a promoter and a second sequence, where the promoter sequence initiates and mediates transcription of the DNA sequence corresponding to the second sequence. In general, “operably linked” or “operatively linked” means that the nucleic acid sequences being linked are contiguous. For example, an operatively linked promoter, enhancer elements, open reading frame, 5′ and 3′ UTR, and terminator sequences result in the accurate production of an RNA molecule. In some aspects, operatively linked nucleic acid elements result in the transcription of an open reading frame and ultimately the production of a polypeptide (i.e., expression of the open reading frame).

The term “regulatory element” is intended to include promoters, enhancers, internal ribosomal entry sites (IRES), and other expression control elements (e.g., transcription termination signals, such as polyadenylation signals and poly-U sequences). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, kidney), or particular cell types (e.g., macrophage, dendritic cell). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I; SV40 enhancer; and the intron sequence between exons 2 and 3 of rabbit β-globin. It will be appreciated by those skilled in the art that the design of the expression vector can depend on such factors as the choice of the host cell to be transformed, the level of expression desired, etc. A vector can be introduced into cells to thereby produce transcripts, proteins, or peptides, encoded by nucleic acids as described herein (e.g., agents that disrupt CK1G2 activity or mutant PKC theta proteins).

The terms “treating” or “treatment” as used herein covers the treatment of a disease in a subject, such as a human, and includes: (i) inhibiting a disease, i.e., arresting its development; (ii) relieving a disease, i.e., causing regression of the disease; (iii) slowing progression of the disease; and/or (iv) inhibiting, relieving, or slowing progression of one or more symptoms of the disease. With respect to a tumor, “treating” or “treatment” also encompasses regression of a tumor, slowing tumor growth, inhibiting metastasis of a tumor, inhibiting relapse or recurrent cancer and/or maintaining remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment can be a continuous prolonged treatment for a chronic disease or a single, or few time administrations for the treatment of an acute condition.

The term “therapeutic” as used herein means a treatment and/or prophylaxis. A therapeutic effect is obtained by suppression, remission, or eradication of a disease state.

### II. Overview

The present disclosure relates to the treatment or prevention of T cell exhaustion, or T cell impairment, in a subject using a PKC theta protein comprising a K413R mutation or an agent that disrupts CK1G2 activity. Alternatively, in some embodiments, the present disclosure relates to the treatment or prevention of T cell exhaustion, or T cell impairment, in a subject using a PKC theta overexpression construct. In certain embodiments, the T cell exhaustion, or T cell impairment, results from chronic conditions, such as chronic viral infection or cancer. In certain embodiments, the T cell exhaustion, or T cell impairment, results from acute respiratory viral infection. In some embodiments, T cell exhaustion, or T cell impairment, occurs following vaccination. In some embodiments, T cell exhaustion, or T cell impairment, occurs during active infection in an individual that has been previously vaccinated.

Adoptive cell transfer (ACT) is a form of immunotherapy that involves the transfer of immune cells into patients. ACT typically involves isolation of lymphocytes from a donor subject, culturing the lymphocytes in vitro to expand the population, and then infusing the lymphocytes into a recipient subject diagnosed with a disease or condition. Lymphocytes used for adoptive transfer can either be derived from spleen cells, from the lymphatics or lymph nodes, or from the blood. In some cases, the isolated lymphocytes are genetically engineered to express antiviral T cell receptors (TCRs) or chimeric antigen receptors (CARs). The lymphocytes used for infusion can be isolated from a donor (allogeneic ACT), or from the disease-bearing host (autologous ACT).

The present disclosure is based, at least in part, on the discovery, that CK1G2 signaling promotes T cell exhaustion whereas PKC theta signaling promotes T cell activation and prevents exhaustion and that a PKC theta K413R mutant protein is degradation resistant and promotes T cell activation and prevents exhaustion, including in cancer and chronic viral infection models. In some embodiments, an agent that disrupts CK1G2 activity or a PKC theta mutant protein provided herein is employed to reverse immune cell exhaustion/impairment. In some embodiments, an agent that disrupts CK1G2 activity or a PKC theta mutant protein provided herein is employed to prevent or ameliorate immune cell exhaustion/impairment.

In one aspect, the present disclosure provides a method for treating or preventing T cell exhaustion, or T cell impairment, in a subject in need thereof, wherein the method comprises administering an effective amount of one or more modified immune cells (e.g., T cells, such as, for example, CD8⁺ T cells) to the subject, wherein the one or more modified immune cells comprise disrupted CK1G2 activity or a PKC theta mutant protein and/or a nucleic acid encoding a PKC theta mutant protein. In some embodiments, the subject is identified as having altered expression of at least one or more immune cell markers associated with T cell exhaustion, or T cell impairment, compared to that observed in a healthy control. In some embodiments, the one or more modified immune cells are derived from immune cells isolated from the subject. In some embodiments, immune cells are isolated from an allogenic donor. The immune cells can be obtained from the peripheral blood, lymph node, spleen, or a tumor. In some embodiments, the immune cells comprise one or more lymphocytes. In some embodiments, the one or more lymphocytes comprise a T cell, a B cell, an NK cell, or any combination thereof. In some embodiments, the one or more lymphocytes comprise a T cell. In some embodiments, the one or more lymphocytes comprise a CD8⁺ T cell. In some embodiments, the one or more lymphocytes comprise one or more exhausted lymphocytes from the subject (e.g., one or more exhausted T cells, for example, one or more exhausted CD8+ T cells). In some embodiments, the one or more lymphocytes do not comprise exhausted lymphocytes but are isolated from a subject having one or more exhausted lymphocytes. In some embodiments, the methods are a form of ACT. In some embodiments, the one or more modified immune cells are engineered to express antiviral or anticancer TCRs or CARs.

In some embodiments, the one or more modified immune cells may be prepared by contacting a population of immune cells (e.g., CD8⁺ T cells) in vitro with an agent that disrupts CK1G2 activity or a nucleotide sequence encoding a mutant PKC theta protein. In some embodiments, the population of immune cells is isolated from the subject. In some embodiments, the population of immune cells is contacted with a plasmid comprising the nucleotide sequence encoding an agent that disrupts CK1G2 activity or a mutant PKC theta protein. In some embodiments, the population of immune cells is contacted with a vector comprising the nucleotide sequence encoding an agent that disrupts CK1G2 activity or a mutant PKC theta protein. In some embodiments, the vector comprises a retrovirus, an adeno-associated virus, a lentivirus, a virus-like particle, polymeric nanoparticle, metallic nanoparticles, or a lipid nanoparticle. In some embodiments, the method may further include expanding the immune cells in vitro prior to contacting the cells with the agent that disrupts CK1G2 activity or the nucleotide sequence encoding a mutant PKC theta protein. In some embodiments, the method may further include expanding the immune cells in vitro following contacting the primary immune cells with the agent that disrupts CK1G2 activity or the nucleotide sequence encoding a mutant PKC theta protein.

In some embodiments, the one or more modified immune cells comprising disrupted CK1G2 activity or the mutant PKC theta protein and/or nucleic acid sequence encoding a mutant PKC theta protein may be administered by any appropriate method, e.g., intravenously, intraperitoneally, subcutaneously, intramuscularly, or intratumorally.

Exemplary sequences encoding mutant PKC theta proteins for use in the methods are provided herein. The mutant PKC theta protein sequences provided are able to produce functional PKC theta protein that is degradation resistant. In some embodiments, the mutant PKC theta protein comprises the amino sequence set forth in SEQ ID NO: 2. In some embodiments, the nucleic acid sequence encoding the mutant PKC theta protein comprises the sequence set forth in SEQ ID NO: 1. Exemplary agents that disrupt CK1G2 activity, such as CRISPR/Cas, meganucleases, TALENs, ZFNs and RNA interference, are described herein. In some embodiments, the agent that disrupts CK1G2 activity is a CRISPR/Cas system.

In some embodiments, the one or more modified immune cells comprise a PKC theta overexpression construct. In some embodiments, the PKC theta overexpression construct encodes a wild type PKC theta protein (i.e., a PKC theta protein lacking mutations). In some embodiments, the PKC theta overexpression construct comprises an open reading frame encoding wild type PKC theta. In some embodiments, the PKC theta overexpression construct comprises the nucleotide sequence as set forth in SEQ ID NO: 3. In some embodiments, the PKC theta overexpression construct encodes a polypeptide comprising the amino acid sequence as set forth in SEQ ID NO: 4. In some embodiments, the one or more modified immune cells comprising a PKC theta overexpression construct are resistant to T cell exhaustion. In some embodiments, the one or more modified immune cells comprising a PKC theta overexpression construct are administered to a subject. In some embodiments, the subject has cancer and/or a microbial infection. In some embodiments, administration of the one or more modified immune cells comprising a PKC theta overexpression construct results in reduced levels of T cell exhaustion as compared to an untreated control subject, increased T cell proliferation, increased T cell viability; and/or increased T cell activity. In some embodiments, the one or more modified immune cells comprising a PKC theta overexpression construct are administered to a subject for the treatment of cancer and/or a chronic microbial infection. In some embodiments, the present disclosure encompasses a kit comprising one or more modified immune cells comprising a PKC theta overexpression construct or a PKC theta overexpression construct, optionally with instructions for use thereof.

In some embodiments, subjects for treatment with a modified immune cell comprising disrupted CK1G2 activity or a mutant PKC theta protein and/or nucleic acid sequence encoding a mutant PKC theta protein provided herein exhibit increased expression one or more immune cell markers, wherein increased or sustained expression of the immune cell marker is associated with T cell exhaustion, or T cell impairment, compared to that observed in a healthy control (e.g., a non-exhausted T cell). In some embodiments, the one or more immune cell markers that are increased is an immune checkpoint protein. In some embodiments, the one or more markers includes PD-1, TIM-3, LAG-3, CTLA-4, TIGIT, and TOX.

In some embodiments, treatment with a modified immune cell comprising disrupted CK1G2 activity or a mutant PKC theta protein and/or nucleic acid sequence encoding a mutant PKC theta protein results in a decrease of at least one or more immune cell markers. In some embodiments, the one or more markers includes PD-1, TIM-3, LAG-3, CTLA-4, TIGIT, and TOX.

In some embodiments, subjects for treatment with a modified immune cell comprising disrupted CK1G2 activity or a mutant PKC theta protein and/or nucleic acid sequence encoding a mutant PKC theta protein provided herein exhibit decreased expression one or more immune cell markers, where decreased expression of the immune cell marker is associated with T cell exhaustion, or T cell impairment, compared to that observed in a healthy control (e.g., a non-exhausted T cell).

In some embodiments, treatment with a modified immune cell comprising disrupted CK1G2 activity or a mutant PKC theta and/or nucleic acid sequence encoding a mutant PKC theta protein results in an increase in cytokine production in the subject compared to that observed prior to administration. In some embodiments, treatment with a modified immune cell comprising disrupted CK1G2 activity or a mutant PKC theta protein and/or nucleic acid sequence encoding a mutant PKC theta protein results in an increase in production of IL-2, TNF, Granzyme B, and/or IFN gamma in the subject compared to that observed prior to administration.

In another aspect, the present disclosure provides a method for treating a chronic microbial infection in a subject in need thereof, wherein the method comprises administering an effective amount of one or more modified immune cells (e.g., CD8⁺ T cells) to the subject, wherein the one or more modified immune cells comprise disrupted CK1G2 activity or a mutant PKC theta protein and/or nucleic acid sequence encoding a mutant PKC theta protein. In some embodiments, the mutant PKC theta protein comprises a K413R mutation. In some embodiments, the agents that disrupts CK1G2 activity is a CRISPR/Cas system, a meganuclease, a TALEN, a ZFN or an RNA interference agent. In some embodiments, the subject is identified as having altered expression of at least one or more immune cell markers associated with T cell exhaustion, or T cell impairment, compared to that observed in a healthy control. In some embodiments, the one or more modified immune cells are derived from immune cells isolated from the subject. In some embodiments, the methods are a form of ACT. In some embodiments, the one or more modified immune cells are engineered to express antiviral or anticancer TCRs or CARs.

In some embodiments, the chronic microbial infection is a bacterial infection, a viral infection, a fungal infection, a protozoan infection, or parasitic infection. In some embodiments, the chronic microbial infection is chronic or latent form of a viral infection. In some embodiments, the chronic microbial infection is caused by a pathogen selected from the group consisting of Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the chronic microbial infection is an antibiotic resistant or antiviral resistant infection (e.g., antibiotic resistant tuberculosis (TB), Methicillin-resistant Staphylococcus aureus (MRSA), Enterovirus 68, Nipah virus, Middle East respiratory syndrome (MERS)).

In some embodiments, the subject having a chronic microbial infection exhibits increased expression one or more immune cell markers, where increased expression of the immune cell marker is associated with T cell exhaustion, or T cell impairment, compared to that observed in a healthy control (e.g., a non-exhausted T cell). In some embodiments, the one or more immune cell markers that are increased is an immune checkpoint protein. In some embodiments, the one or more markers includes PD-1, TIM-3, LAG-3, CTLA-4, TIGIT, and TOX.

In some embodiments, the subject having a chronic microbial infection exhibits decreased expression one or more immune cell markers, where decreased expression of the immune cell marker is associated with T cell exhaustion, or T cell impairment, compared to that observed in a healthy control (e.g., a non-exhausted T cell). In some embodiments, the one or more markers includes PD-1, TIM-3, LAG-3, CTLA-4, TIGIT, and TOX.

In any of the methods provided here, subjects for treatment with a modified immune cell comprising disrupted CK1G2 activity or a mutant PKC theta protein or nucleic acid sequence encoding a mutant PKC theta protein provided herein can be a human or a non-human animal.

In another aspect, the present disclosure provides a method for treating a cancer in a subject in need thereof, wherein the method comprises administering an effective amount of one or more modified immune cells (e.g., CD8⁺ T cells) to the subject, wherein the one or more modified immune cells comprise disrupted CK1G2 activity or a mutant PKC theta protein or nucleic acid sequence encoding a mutant PKC theta protein, where the subject is identified as having altered expression of at least one or more immune cell markers associated with T cell exhaustion, or T cell impairment, compared to that observed in a healthy control. In some embodiments, the mutant PKC theta protein comprises a K413R mutation. In some embodiments, CK1G2 activity was disrupted using an agent that disrupts CK1G2 activity (e.g., a CRISPR/Cas system). In some embodiments, the one or more modified immune cells are derived from immune cells isolated from the subject. In some embodiments, the cancer is a glioblastoma, a leukemia, or a non-small cell lung cancer. In some embodiments, the cancer is a metastatic cancer. In some embodiments, the cancer is a carcinoma, adenoma, adenocarcinoma, blastoma, sarcoma, or lymphoma. In some embodiments, the cancer is a basal cell carcinoma, biliary tract cancer, bladder cancer, breast cancer, cervical cancer, choriocarcinoma, CNS cancer, colon cancer, colorectal cancer, connective tissue cancer, cancer of the digestive system, endometrial cancer, esophageal cancer, eye cancer, gastric cancer, glial cell tumor, head and neck cancer, hepatoma, hepatic carcinoma, Hodgkin's lymphoma, Non-Hodgkin's lymphoma, intra-epithelial neoplasm, kidney cancer, larynx cancer, liver cancer, small-cell lung cancer, non-small cell lung cancer, melanoma, myeloma, neuroblastoma, oral cavity cancer, ovarian cancer, pancreatic cancer, prostate cancer, rectal cancer, renal cancer, cancer of the respiratory system, retinoblastoma, rhabdomyosarcoma, salivary gland carcinoma, squamous cell cancer, stomach cancer, testicular cancer, thyroid cancer, uterine cancer, cancer of the urinary system, or a vulvar cancer. In some embodiments, the immune cells are obtained from a donor subject having solid tumor where the subject is identified as having altered expression of at least one or more immune cell markers associated with T cell exhaustion, or T cell impairment, compared to that observed in a healthy control. In some embodiments, the solid tumor is a metastatic tumor. In some embodiments, the methods are a form of ACT. In some embodiments, the one or more modified immune cells are engineered to express antiviral or anticancer TCRs or CARs.

As is demonstrated below in Table B, the PKC eta, PKC theta, and CK1G2 genes are highly conserved between mice and humans. Accordingly, the results described herein relating to the murine PKC eta, PKC theta, and CK1G2 genes reasonably correlate to the equivalent human genes.

PRKCH (PKC eta) alignment between Mus musculus (mouse)
and Homo sapiens (human)
97.8% identity
99.3% similarity
Sequences downloaded from Uniprot
Alignment performed using Smith-Waterman algorithm in Snapgene
mouse 1
MSSGTMKFNGYLRVRIGEAVGLQPTRWSLRHSLFKKGHQLLDPYLTVSVDQVRVG
QTSTKQKTNKPTYNE
human 1
MSSGTMKFNGYLRVRIGEAVGLQPTRWSLRHSLFKKGHQLLDPYLTVSVDQVRVG
QTSTKQKTNKPTYNE
mouse 71
EFCANVTDGGHLELAVFHETPLGYDHFVANCTLQFQELLRTAGTSDTFEGWVDLEP
EGKVFVVITLTGSF
human 71
EFCANVTDGGHLELAVFHETPLGYDHFVANCTLQFQELLRTTGASDTFEGWVDLE
PEGKVFVVITLTGSF
mouse 141
TEATLQRDRIFKHFTRKRQRAMRRRVHQVNGHKFMATYLRQPTYCSHCREFIWGV
FGKQGYQCQVCTCVV
human 141
TEATLQRDRIFKHFTRKRQRAMRRRVHQINGHKFMATYLRQPTYCSHCREFIWGV
FGKQGYQCQVCTCVV
mouse 211
HKRCHHLIVTACTCQNNINKVDAKIAEQRFGINIPHKFNVHNYKVPTFCDHCGSLL
WGIMRQGLQCKICK
human 211
HKRCHHLIVTACTCQNNINKVDSKIAEQRFGINIPHKFSIHNYKVPTFCDHCGSLLW
GIMRQGLQCKICK
mouse 281
MNVHIRCQANVAPNCGVNAVELAKTLAGMGLQPGNISPTSKLISRSTLRRQGKEGS
KEGNGIGVNSSSRF
human 281
MNVHIRCQANVAPNCGVNAVELAKTLAGMGLQPGNISPTSKLVSRSTLRRQGKES
SKEGNGIGVNSSNRL
mouse 351
GIDNFEFIRVLGKGSFGKVMLARIKETGELYAVKVLKKDVILQDDDVECTMTEKRI
LSLARNHPFLTQLF
human 351
GIDNFEFIRVLGKGSFGKVMLARVKETGDLYAVKVLKKDVILQDDDVECTMTEKRI
LSLARNHPFLTQLF
mouse 421
CCFQTPDRLFFVMEFVNGGDLMFHIQKSRRFDEARARFYAAEIISALMFLHEKGIIY
RDLKLDNVLLDHE
human 421
CCFQTPDRLFFVMEFVNGGDLMFHIQKSRRFDEARARFYAAEIISALMFLHDKGIIY
RDLKLDNVLLDHE
mouse 491
GHCKLADFGMCKEGICNGVTTATFCGTPDYIAPEILQEMLYGPAVDWWAMGVLLY
EMLCGHAPFEAENED
human 491
GHCKLADFGMCKEGICNGVTTATFCGTPDYIAPEILQEMLYGPAVDWWAMGVLLY
EMLCGHAPFEAENED
mouse 561
DLFEAILNDEVVYPTWLHEDATGILKSFMTKNPTMRLGSLTQGGEHEILRHPFFKEI
DWAQLNHRQLEPP
human 561
DLFEAILNDEVVYPTWLHEDATGILKSFMTKNPTMRLGSLTQGGEHAILRHPFFKEI
DWAQLNHR QIEPP
mouse 631
(SEQ ID NO: 10)
FRPRIKSREDVSNFDPDFIKEEPVLTPIDEGHLPMINQDEFRNFSYVSPELQ
human 631
(SEQ ID NO: 11)
FRPRIKSREDVSNFDPDFIKEEPVLTPIDEGHLPMINQDEFRNFSYVSPELQ
PRKCQ (PKC theta) alignment between Mus musculus (mouse) and
Homo sapiens (human), with conserved K413 residue bold
and underlined
94.9% identity
97.7% similarity
Sequences downloaded from Uniprot
Alignment performed using Smith-Waterman algorithm in Snapgene
mouse 1
MSPFLRIGLSNFDCGTCQACQGEAVNPYCAVLVKEYVESENGQMYIQKKPTMYPP
WDSTFDAHINKGRVM
human 1
MSPFLRIGLSNFDCGSCQSCQGEAVNPYCAVLVKEYVESENGQMYIQKKPTMYPP
WDSTFDAHINKGRVM
mouse 71
QIIVKGKNVDLISETTVELYSLAERCRKNNGRTEIWLELKPQGRMLMNARYFLEMS
DTKDMSEFENEGFF
human 71
QIIVKGKNVDLISETTVELYSLAERCRKNNGKTEIWLELKPQGRMLMNARYFLEM
SDTKDMNEFETEGFF
mouse 141
ALHQRRGAIKQAKVHHVKCHEFTATFFPQPTFCSVCHEFVWGLNKQGYQCRQCN
AAIHKKCIDKVIAKCT
human 141
ALHQRRGAIKQAKVHHVKCHEFTATFFPQPTFCSVCHEFVWGLNKQGYQCRQCN
AAIHKKCIDKVIAKCT
mouse 211
GSAINSRETMFHKERFKIDMPHRFKVYNYKSPTFCEHCGTLLWGLARQGLKCDAC
GMNVHHRCQTKVANL
human 211
GSAINSRETMFHKERFKIDMPHRFKVYNYKSPTFCEHCGTLLWGLARQGLKCDAC
GMNVHHRCQTKVANL
mouse 281
CGINQKLMAEALAMIESTQQARSLRDSEHIFREGPVEIGLPCSTKNETRPPCVPTPG
KREPQGISWDSPL
human 281
CGINQKLMAEALAMIESTQQARCLRDTEQIFREGPVEIGLPCSIKNEARPPCLPTPG
KREPQGISWESPL
mouse 351
DGSNKSAGPPEPEVSMRRTSLQLKLKIDDFILHKMLGKGSFGKVFLAEFKRTNQFF
AIKALKKDVVLMDD
human 351
DEVDKMCHLPEPELNKERPSLQIKLKIEDFILHKMLGKGSFGKVFLAEFKKTNQFF
AIKALKKDVVLMDD
mouse 421
DVECTMVEKRVLSLAWEHPFLTHMFCTFQTKENLFFVMEYLNGGDLMYHIQSCH
KFDLSRATFYAAEVIL
human 421
DVECTMVEKRVLSLAWEHPFLTHMFCTFQTKENLFFVMEYLNGGDLMYHIQSCH
KFDLSRATFYAAEIIL
mouse 491
GLQFLHSKGIVYRDLKLDNILLDRDGHIKIADFGMCKENMLGDAKTNTFCGTPDYI
APEILLGQKYNHSV
human 491
GLQFLHSKGIVYRDLKLDNILLDKDGHIKIADFGMCKENMLGDAKTNTFCGTPDYI
APEILLGQKYNHSV
mouse 561
DWWSFGVLVYEMLIGQSPFHGQDEEELFHSIRMDNPFYPRWLEREAKDLLVKLFV
REPEKRLGVRGDIRQ
human 561
DWWSFGVLLYEMLIGQSPFHGQDEEELFHSIRMDNPFYPRWLEKEAKDLLVKLFV
REPEKRLGVRGDIRQ
mouse 631
HPLFREINWEELERKEIDPPFRPKVKSPYDCSNFDKEFLSEKPRLSFADRALINSMD
QNMFSNFSFINPG
human 631
HPLFREINWEELERKEIDPPFRPKVKSPFDCSNFDKEFLNEKPRLSFADRALINSMD
QNMFRNFSFMNPG
mouse 701
(SEQ ID NO: 12)
METLI
human 701
(SEQ ID NO: 13)
MERLI
CSNK1G2 (Casein kinase 1 G2) alignment between Mus musculus
(mouse) and Homo sapiens (human)
95.2% identity
97.1% similarity
Sequences downloaded from Uniprot Jun. 24, 2025
Alignment performed using Smith-Waterman algorithm in Snapgene
mouse 1
MDFDKKGGKGELEEGRRMSKTGTSRSNHGVRSSGTSSGVLMVGPNFRVGKKIGC
GNFGELRLGKNLYTNE
human 1
MDFDKKGGKGETEEGRRMSKAGGGRSSHGIRSSGTSSGVLMVGPNFRVGKKIGC
GNFGELRLGKNLYTNE
mouse 71
YVAIKLEPIKSRAPQLHLEYRFYKQLSTTEGVPQVYYFGPCGKYNAMVLELLGPSL
EDLFDLCDRTFTLK
human 71
YVAIKLEPIKSRAPQLHLEYRFYKQLSATEGVPQVYYFGPCGKYNAMVLELLGPSL
EDLFDLCDRTFTLK
mouse 141
TVLMIAIQLITRMEYVHTKSLIYRDVKPENFLVGRPGSKRQHSIHIIDFGLAKEYIDP
ETKKHIPYREHK
human 141
TVLMIAIQLITRMEYVHTKSLIYRDVKPENFLVGRPGTKRQHAIHIIDFGLAKEYIDP
ETKKHIPYREHK
mouse 211
SLTGTARYMSINTHLGKEQSRRDDLEALGHMFMYFLRGSLPWQGLKADTLKERYQ
KIGDTKRATPIEVLC
human 211
SLTGTARYMSINTHLGKEQSRRDDLEALGHMFMYFLRGSLPWQGLKADTLKERYQ
KIGDTKRATPIEVLC
mouse 281
ESFPEEMATYLRYVRRLDFFEKPDYDYLRKLFTDLFDRSGYVFDYEYDWAGKPLP
TPIGTVHPDVPSQPP
human 281
ENFPEEMATYLRYVRRLDFFEKPDYDYLRKLFTDLFDRSGFVFDYEYDWAGKPLP
TPIGTVHTDLPSQPQ
mouse 351
(SEQ ID NO: 14)
HRDKAQLHTKNQALNSTNGELNTDDPTAGHSNAPIAAPAEVEVADETKCCCFFKR
RKRKSLQRHK
human 351
(SEQ ID NO: 15)
LRDKTQPHSKNQALNSTNGELNADDPTAGHSNAPITAPAEVEVADETKCCCFFKRR
KRKSLQRHK

### III. Tools for Genetic Manipulation

The present technology contemplates methods and compositions comprising agents for disrupting CK1G2 activity. In particular, the present technology relates to agents for disrupting CK1G2 activity comprising targeted genome engineering (also known as genome editing) and RNA interference (RNAi) methods and compositions. Provided herein are methods and compositions for using RNAi or modifying a target genomic locus in a cell to modulate the expression of a CK1G2 gene. Targeted genome engineering techniques described herein include the CRISPR (clustered regularly interspaced short palindromic repeats)/Cas (CRISPR-associated) system, meganucleases, zinc finger nucleases (ZFNs), and TAL effector nucleases (TALENs). Such techniques may be employed to bind to and/or cleave a genomic region of interest of or adjacent to a CK1G2 gene. In some embodiments, the agent that disrupts CK1G2 activity comprises a therapeutic nucleic acid or oligonucleotide selected from an antisense oligonucleotide (ASO), an aptamer, an siRNA, a shRNA, a miRNA, a gRNA, or an sgRNA. In some embodiments, therapeutic nucleic acids or oligonucleotides are codon optimized for enhanced expression and efficacy in a chosen subject species. In some embodiments, the genome editing techniques described herein generate a specific sequence change or gene edit (e.g., insertion, deletion, or substitution) in the 5′-UTR of a CK1G2 gene, such as generating a single nucleotide gene edit to form an out-of-frame start codon upstream of the gene's ORF, thereby suppressing expression of the CK1G2 gene. In some embodiments, the gene edit (e.g., deletion, insertion, or substitution) results in production of an upstream, out-of-frame start codon that may result in the elimination of protein production or a nonfunctional protein. In some embodiments, the genome editing techniques described herein generate a specific sequence change or gene edit (e.g., insertion, deletion, or substitution) in the coding region or a non-coding region of a CK1G2 gene, such as generating a large deletion to form (1) an out-of-frame start codon upstream of the gene's ORF, thereby suppressing expression of the CK1G2 gene, or (2) a non-functional protein product resulting from a frame shift downstream of the gene edit. In some embodiments, the large deletion is greater than 50 bases, greater than 100 bases, greater than 200 bases, greater than 500 bases, greater than 1000 bases, greater than 2000 bases, greater than 5000 bases, or greater than 10000 bases. In some embodiments, the large deletion is generated in a CK1G2 gene. RNAi techniques include anti-sense oligonucleotides (ASOs), sense co-suppression, microRNA (miRNA), short interfering RNA (siRNA), and short hairpin RNA (shRNA).

Delivery systems or carriers/vehicles for oligonucleotide agents of the present technology are well known in the art. In some embodiments, oligonucleotides may be delivered to a cell by means of a viral vector (such as an adeno-associated virus (AAV) or lentivirus) and/or particle and/or nanoparticle delivery (such as a virus-like particle (VLP) or lipid nanoparticle (LNP)). Thus, in some embodiments, oligonucleotides used in any one or more of the methods for disrupting CK1G2 activity described herein may be formulated in a carrier, such as, but not limited to, an AAV, a VLP, or an LNP. In some embodiments, immune cells are contacted with the agent for disrupting CK1G2 activity formulated in a carrier.

### CRISPR Cas Systems

In some embodiments, the methods of the present technology relate to the use of a CRISPR/Cas system that binds to a target site in a region of interest in a genome (e.g., a CK1G2 gene), wherein the CRISPR/Cas system comprises a CRISPR/Cas nuclease and an engineered crRNA/tracrRNA (or single guide RNA (sgRNA) or guide RNA (gRNA)). In some embodiments, the CRISPR system generally comprises (i) a polynucleotide encoding a Cas protein, and (ii) at least one sgRNA for RNA-guided genome engineering.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Cys3, Cse1, Cse2, Csc1, Csc2, Csa5, Csn2, Csm1, Csm2, Csm3, Csm4, Csm5, Csm6, Smr1, Cmr3, Cmr4, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein is a Streptococcus pyogenes Cas9 protein. In some embodiments, the Cas protein is a Cas12a (Cpf1) protein. In some embodiments, the Cas protein is a Csm1 protein. These enzymes are known. For example, the amino acid sequence of S. pyogenes Cas9 protein may be found in the SwissProt database under accession number Q99ZW2. The amino acid sequence of Francisella tularensis subsp. Novicida Cpf1 protein may be found in the UniProt database under accession number A0Q7Q2. The amino acid sequence of Thermococcus onnurineus Csm1 protein may be found in the UniProt database under accession number B6YWB8.

The sgRNA molecules comprise a crRNA-tracrRNA scaffold polynucleotide and a targeting sequence corresponding to a genomic target of interest.

In some embodiments, the CRISPR/Cas system recognizes a target site in a CK1G2 gene. In some embodiments, the CRISPR/Cas system recognizes a target in one or more regulatory elements that impact expression of a CK1G2 gene. The CRISPR/Cas system as described herein may bind to and/or cleave the region of interest in a region upstream of the coding region of a CK1G2 gene. In some embodiments, the CRISPR/Cas system generates a specific sequence change in the 5′-UTR of a CK1G2 gene, such as generating a single nucleotide gene edit to form an out-of-frame start codon upstream of the gene's ORF. In some embodiments, the gene edit (e.g., deletion, insertion, or substitution) results in production of an upstream, out-of-frame start codon that may result in the elimination of protein production or a nonfunctional protein. In some embodiments, the CRISPR/Cas system generates a specific sequence change or gene edit (e.g., insertion, deletion, or substitution) in the coding region or a non-coding region of a CK1G2 gene, such as generating a large deletion to form (1) an out-of-frame start codon upstream of the gene's ORF, thereby suppressing expression of the CK1G2 gene, or (2) a non-functional protein product resulting from a frame shift downstream of the gene edit. In some embodiments, the large deletion is greater than 50 bases, greater than 100 bases, greater than 200 bases, greater than 500 bases, greater than 1000 bases, greater than 2000 bases, greater than 5000 bases, or greater than 10000 bases.

The CRISPR/Cas system can be based on the Cas9 nuclease and an engineered single guide RNA (sgRNA) that specifies the targeted nucleic acid sequence. Cas9 is a large monomeric DNA nuclease guided to a DNA target sequence adjacent to the PAM (protospacer adjacent motif) sequence motif by a complex of two non-coding RNAs: CRISPR RNA (crRNA) and trans-activating crRNA (tracrRNA). The precise sequence and length requirements for the PAM differ depending on the CRISPR enzyme used, but PAMs are typically 2-5 base pair sequences adjacent the protospacer (that is, the target sequence). Typically, in currently characterized CRISPR-Cas systems, there are two requirements for DNA interference: (i) the target sequence has to match one of the spacers present in the respective CRISPR array, and (ii) the target sequence complementary to the spacer (protospacer) has to be flanked by the appropriate PAM.

The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can induce site-specific double strand breaks (DSBs) into genomic DNA of live cells. See, e.g., Mussolino, Nat. Biothechnol., 31:208-209 (2013). In some embodiments, the Cas9 protein is expressed in a cell as a fusion to a nuclear localization signal (NLS) to ensure delivery into nuclei. In some embodiments, the Cas9 protein is tagged (e.g., FLAG- or GFP-tagged). In some embodiments, promoters may be used to drive Cas9 expression in a cell. In some embodiments, the Cas9 enzyme is S. pneumoniae, S. pyogenes, or S. thermophiles Cas9, and may include mutated Cas9 derived from these organisms. The enzyme may be a Cas9 homolog or ortholog. In some embodiments, the CRISPR enzyme (e.g., Cas9 enzyme) is codon-optimized for expression in a mammalian cell, such as a human cell.

The CRISPR/Cas system can be based on the Cpf1 nuclease and an engineered single guide RNA (sgRNA) that specifies the targeted nucleic acid sequence. Cpf1 is distinguished from Cas9 by a its single RuvC endonuclease active site, its 5′ protospacer adjacent motif preference, and for creating sticky rather than blunt ends at the cut site. The Cpf1 protein has a RuvC-like endonuclease domain that is similar to the RuvC domain of Cas9. Cpf1 does not have a HNH endonuclease domain, and the N-terminal of Cpf1 does not have an alpha-helical recognition lobe, unlike Cas9. In some embodiments, the Cpf1 protein is tagged (e.g., FLAG- or GFP-tagged). In some embodiments, promoters may be used to drive Cpf1 expression in a cell. In some embodiments, the Cpf1 enzyme is Francisella tularensis subsp. Novicida Cpf1, and may include mutated Cpf1 derived from these organisms. The enzyme may be a Cpf1 homolog or ortholog. In some embodiments, the CRISPR enzyme (e.g., Cpf1 enzyme) is codon-optimized for expression in a mammalian cell, such as a human cell.

The CRISPR/Cas system can be based on the Csm1 nuclease and an engineered single guide RNA (sgRNA) that specifies the targeted nucleic acid sequence. Csm1 belongs to the Cas10 family of endonucleases. Csm1 is the largest subunit of the Csm interference complex in the type III-A CRISPR system. Csm1 exhibits ssDNA-specific endo- and exonuclease activity. In some embodiments, promoters may be used to drive Csm1 expression in a cell. In some embodiments, the Csm1 enzyme is Thermococcus onnurineus Csm1, and may include mutated Csm1 derived from these organisms. The enzyme may be a Csm1 homolog or ortholog. In some embodiments, the CRISPR enzyme (e.g., Csm1 enzyme) is codon-optimized for expression in a mammalian cell, such as a human cell.

The single guide RNA (sgRNA) is the second component of the CRISPR/Cas system that forms a complex with a Cas nuclease. The sgRNA is created by fusing crRNA with tracrRNA. The sgRNA guide sequence located at the 5′ end confers DNA target specificity. By modifying the guide sequence, sgRNAs with different target specificities can be designed to target any desired endogenous gene. In some embodiments, the target sequence is about 1,000, about 975, about 950, about 925, about 900, about 875, about 850, about 825, about 800, about 775, about 750, about 725, about 700, about 675, about 650, about 625, about 600, about 575, about 550, about 525, about 500, about 475, about 450, about 425, about 400, about 375, about 350, about 325, about 300, about 275, about 250, about 225, about 200, about 175, about 150, about 125, about 100, about 90, about 80, about 70, about 60, about 50, about 40, about 30, about 20, or about 15 base pairs upstream of the transcription start site, or the target sequence may be any number of base pairs in-between these values upstream of the transcription start site. In some embodiments, the target sequence is about 1 to about 10 base pairs upstream of the transcription start site (e.g., positions −10, −9, −8, −7, −6, −5, −4, −3, −2, or −1). In some embodiments, the target sequence is located within the open reading frame of the gene of interest. In some embodiments, the target sequence is located within a coding region of the gene of interest.

In some embodiments, the CRISPR/Cas system comprises at least two sgRNAs. In some embodiments, a target sequence of at least one of the at least two sgRNAs is about 1,000, about 975, about 950, about 925, about 900, about 875, about 850, about 825, about 800, about 775, about 750, about 725, about 700, about 675, about 650, about 625, about 600, about 575, about 550, about 525, about 500, about 475, about 450, about 425, about 400, about 375, about 350, about 325, about 300, about 275, about 250, about 225, about 200, about 175, about 150, about 125, about 100, about 90, about 80, about 70, about 60, about 50, about 40, about 30, about 20, or about 15 base pairs upstream of the transcription start site, or the target sequence may be any number of base pairs in-between these values upstream of the transcription start site. In some embodiments, the target sequence of at least one of the at least two sgRNAs is about 1 to about 10 base pairs upstream of the transcription start site (e.g., positions −10, −9, −8, −7, −6, −5, −4, −3, −2, or −1). In some embodiments, the target sequence of at least one of the at least two sgRNAs is located within the open reading frame of the gene of interest. In some embodiments, the target sequence of at least one of the at least two sgRNAs is located within a coding region of the gene of interest. In some embodiments, the target sequences of at least two of the at least two sgRNAs are located within the open reading frame of the gene of interest. In some embodiments, the target sequences of at least two of the at least two sgRNAs are located within a coding region of the gene of interest. In some embodiments, the CRISPR/Cas system comprises two sgRNAs, wherein the two sgRNAs have non-overlapping target sequences. In some embodiments, the target sequences of the two sgRNAs are separated by at least 50 bases, at least 100 bases, at least 200 bases, at least 500 bases, at least 1000 bases, at least 2000 bases, at least 5000 bases, or at least 10000 bases.

It is not intended that the present technology be limited to any particular distance restraint with regard to the location of the guide RNA target sequence from the gene transcription start site. In some embodiments, the target sequence lies “in proximity to” a gene of interest, where “in proximity to” refers to any distance from the gene of interest, wherein the Cas-regulatory domain fusion is able to exert an effect on gene expression. In some embodiments, the target sequence lies upstream of the ORF of the gene of interest.

The canonical length of the guide sequence is about 20 bp and the DNA target sequence is about 20 bp followed by a PAM sequence having the consensus NGG sequence, or other derivate CRISPR systems such as those with alternative PAMs, including NG¹. In some embodiments, sgRNAs are expressed in a cell using RNA polymerase promoters.

When the DSBs are repaired by either NHEJ or HDR, the sequence at the repair site can be modified or new genetic information can be inserted (e.g., donor DNA comprising a desired gene edit can be inserted into the target gene at the break site). Although HDR typically occurs at lower and more variable frequencies than NHEJ, it can be leveraged to generate precise, defined modifications at a target locus in the presence of an exogenously introduced repair template. Accordingly, exogenous repair templates, designed by methods known in the art, can also be delivered into a cell, most often in the form of a synthetic, single-stranded DNA donor oligo or DNA donor plasmid, to generate a precise change in the genome. Single-stranded DNA donor oligos are delivered into a cell to insert or change short sequences (SNPs, amino acid substitutions, epitope tags, etc.) of DNA in the endogenous genomic target region. The benefits of using a synthetic DNA donor oligo is that no cloning is required to generate the donor template and DNA modifications can be added during synthesis for different applications, such as increased resistance to nucleases. Traditionally, the maximum insert length recommended for use with a DNA donor oligo is about 50 nucleotides.

In some embodiments, the present technology provides an engineered, programmable, non-naturally occurring CRISPR/Cas system comprising a Cas9 protein and one or more single guide RNAs (sgRNAs) that target the genomic loci of DNA molecules encoding a CK1G2 gene, and the Cas9 protein cleaves the genomic loci of the DNA molecules encoding the one or more gene products, whereby expression of the one or more gene products is altered. In some embodiments, Cas9 introduces multiple DSBs in the same cell (i.e., multiplexes) via expression of one or more distinct guide RNAs.

In some embodiments, the present technology provides a method for targeted genomic modification of cells to alter the expression of a CK1G2 gene, the method comprising introducing into a cell, comprising and expressing a DNA molecule having a target sequence and encoding the CK1G2 gene involved, an engineered CRISPR/Cas system comprising (a) an expression construct comprising a first polynucleotide encoding a Cas9 protein, or a variant thereof or a fusion protein therewith, and a second polynucleotide encoding a guide RNA comprising: (i) a crRNA-tracrRNA scaffold polynucleotide, and (ii) a targeting sequence operably linked to the crRNA-tracrRNA scaffold polynucleotide, where the targeting sequence corresponds to a genomic locus of interest, and (b) delivering the expression construct into the cell, where the first and second polynucleotides are expressed (transcribed) within the cell. This method can optionally further include visualizing, identifying, or selecting for cells having a genomic modification at the genomic locus of interest that is induced by the delivering the expression construct into the cell.

In some embodiments of the methods of the present technology, the Cas9 polypeptide and one or more guide RNA are encoded on a single vector. In some embodiments, the single vector is a plasmid. In some embodiments of the methods of the present technology, the Cas9 polypeptide and the one or more guide RNA are encoded on two separate vectors. In these methods, the steps generally follow the sequence of introducing into a cell containing and expressing a DNA molecule having a target sequence and encoding the CK1G2 gene an engineered CRISPR/Cas system comprising (a) a Cas9 polynucleotide or a conservative variant thereof, and a guide RNA comprising (i) a crRNA-tracrRNA scaffold polynucleotide, and (ii) a targeting sequence operably linked to the crRNA-tracrRNA scaffold polynucleotide, with the targeting sequence corresponding to a genomic locus of interest, and (b) delivering the two polynucleotides into the cell. In variations of this method, a donor polynucleotide having homology to the genomic target of interest is included in a co-transfection. In some variations of these methods, the transfected material can be either plasmid DNA or RNA generated by in vitro transcription. In still other variations, the methods for targeted genomic modification are multiplexed, meaning that more than one genomic locus is targeted for modification. In still other variations of these methods, the transformation of the cells can be followed by visualizing, identifying, or selecting for cells having a genomic modification at the genomic locus of interest.

### Meganucleases

In some embodiments, the compositions and methods described herein employ a meganuclease DNA binding domain for binding to a region of interest in the genome of a cell. Meganucleases are engineered versions of naturally occurring restriction enzymes that typically have extended DNA recognition sequences (e.g., about 14 to about 40 base pairs in length). Meganucleases (also known as homing endonucleases) are commonly grouped into five families based on sequence and structure motifs: the LAGLIDADG (SEQ ID NO: 16) family, the GIY-YIG family, the His-Cyst box family, the PD-(D/E)XK family, and the HNH family. In some embodiments, the meganuclease comprises an engineered homing endonuclease. The recognition sequences of homing endonucleases and meganucleases such as I-Sce, I-CeuI, PI-PspI, PI-Sce, I-SceIV, I-CsmI, I-PanI, I-SceII, I-PpoI, I-SceIII, I-CreI, I-TevI, I-TevII, and I-TevIII are known.

In some embodiments, the meganuclease is tailored to recognize a target in a CK1G2 gene. The meganucleases as described herein may bind to and/or cleave the region of interest in a region upstream of the coding region of a CK1G2 gene. Gene insertion or correction can be achieved by the introduction of a DNA repair matrix containing sequences homologous to the endogenous sequence surrounding the DNA break. Gene edits can be created either at or distal to the break. In some embodiments, the meganuclease generates a specific sequence change in the 5′-UTR of a CK1G2 gene, such as generating a single nucleotide gene edit to form an out-of-frame start codon upstream of the gene's ORF.

### TALENs

In some embodiments, the compositions and methods described herein employ transcription activator-like effector nucleases (TALENs) to edit genomes by inducing double-strand breaks (DSBs). TALENs are restriction enzymes that can be engineered to cleave specific sequences of DNA. TALENs are constructed by fusing a TAL effector DNA-binding domain to a DNA cleavage domain (e.g., a nuclease domain such as that derived from the FokI endonuclease). Transcription activator-like effectors (TALEs) can be engineered according to methods known in the art to bind to a desired DNA sequence, and when combined with a nuclease, provide a technique for cutting DNA at specific locations. For example, after a target sequence in a CK1G2 gene is identified, a corresponding TALEN sequence is engineered and inserted into a plasmid. The plasmid is inserted into a target cell where it is translated to produce a functional TALEN, which then enters the nucleus where it binds to and cleaves its target sequence. Such an approach can be employed to introduce an exogenous DNA sequence into the target gene as the DSB is being repaired through either homology-directed repair or non-homologous end-joining. For example, in some embodiments, the use of TALEN technology generates a specific sequence change (e.g., insertion, deletion, or substitution) in the 5′-UTR of a CK1G2 gene, resulting in the production of an out-of-frame start codon upstream of the gene's ORF.

### ZFNs

In some embodiments, the compositions and methods described herein employ zinc finger nucleases (ZFNs) to edit genomes by inducing double-strand breaks (DSBs). ZFNs are artificial restriction enzymes generated by fusing a zinc finder DNA-binding domain to a DNA cleavage domain (e.g., a nuclease domain such as that derived from the FokI endonuclease). ZFNs can be engineered to bind and cleave DNA at specific locations. ZFNs contain two protein domains. The first domain is the DNA-binding domain, which contains eukaryotic transcription factors and the zinc finger. The second domain is a nuclease domain that contains the FokI restriction enzyme responsible for cleaving DNA. ZFNs can be engineered according to methods known in the art to bind to a desired DNA sequence and cleave DNA at specific locations. For example, after a target sequence in a CK1G2 gene is identified, a corresponding ZFN sequence is engineered and inserted into a plasmid. The plasmid is inserted into a target cell where it is translated to produce a functional ZFN, which then enters the nucleus where it binds to and cleaves its target sequence introducing a double strand break (DSB). Such an approach can be employed to introduce an exogenous DNA sequence into the target gene as the DSB is being repaired through either homology-directed repair or non-homologous end-joining. For example, in some embodiments, the use of ZFN technology generates a specific sequence change in the 5′-UTR of a CK1G2 gene, such as the insertion of an out-of-frame start codon upstream of the gene's ORF.

### RNA Interference

In one aspect of the invention, methods and constructs are provided for suppressing a CK1G2 gene. While any method may be used for suppressing a CK1G2 gene, the present invention contemplates anti-sense, sense co-suppression, microRNA (miRNA), short interfering RNA (siRNA), and short hairpin RNA (shRNA).

shRNA techniques involve stable transformation using shRNA plasmid constructs (Helliwell and Waterhouse, Methods Enzymol. 392:24-35 (2005)). Such plasmids are composed of a fragment of the target gene to be silenced in an inverted repeat structure. The inverted repeats are separated by a spacer, often an intron. The RNAi construct driven by a suitable promoter is integrated into the genome and subsequent transcription of the transgene leads to an RNA molecule that folds back on itself to form a double-stranded hairpin RNA. Non-limiting examples of suitable promoters include RNA Pol III promoter (such as U6 or H1) or an RNA Pol II promoter (such as CMV). This double-stranded RNA structure is recognized by the cell and cut into small RNAs (about 21 nucleotides long) called small interfering RNAs (siRNAs). siRNAs associate with a protein complex (RISC) which goes on to direct degradation of the mRNA for the target gene.

microRNA (miRNA) techniques exploit the miRNA pathway that functions to silence endogenous genes. In this method, 21 nucleotide long fragments of the gene to be silenced are introduced into a pre-miRNA gene to form a pre-miRNA construct. The pre-miRNA construct is transferred into the genome using transformation methods apparent to one skilled in the art. After transcription of the pre-miRNA, processing yields miRNAs that target genes, which share nucleotide identity with the 21 nucleotide miRNA sequence.

In RNAi silencing techniques, several factors can influence the choice of length of the fragment. The shorter the fragment the less frequently effective silencing will be achieved, but very long hairpins increase the chance of recombination. The effectiveness of silencing also appears to be gene dependent and could reflect accessibility of target mRNA or the relative abundances of the target mRNA and the hpRNA in cells in which the gene is active. A fragment length of between 100 and 800 bp, preferably between 300 and 600 bp, is generally suitable to maximize the efficiency of silencing obtained. The other consideration is the part of the gene to be targeted. 5′ UTR, coding region, and 3′ UTR fragments can be used with equally good results. As the mechanism of silencing depends on sequence homology there is potential for cross-silencing of related mRNA sequences. Where this is not desirable a region with low sequence similarity to other sequences, such as a 5′ or 3′ UTR, should be chosen. The rule for avoiding cross-homology silencing appears to be to use sequences that do not have blocks of sequence identity of over 20 bases between the construct and the non-target gene sequences.

Antisense techniques involve introducing into a cell an antisense oligonucleotide (ASO) that will bind to the messenger RNA (mRNA) produced by the gene of interest. The “antisense” oligonucleotide has a base sequence complementary to the gene's messenger RNA (mRNA), which is called the “sense” sequence. Activity of the sense segment of the mRNA is blocked by the anti-sense mRNA segment, thereby effectively inactivating gene expression. Sense co-suppression techniques involve introducing a highly expressed sense transgene into a cell resulting in reduced expression of both the transgene and the endogenous gene. The effect depends on sequence identity between transgene and endogenous gene.

### IV. Methods of Obtaining and Preparing Immune Cells for Transfer

Immune cells for use in the methods provided herein can be obtained using any suitable method known in the art. In some embodiments, methods of the present technology are ACT methods. In some embodiments, the ACT methods are TIL therapies. In some embodiments, TIL therapies comprise isolating tumor infiltrating lymphocytes from a subject's tumor for use in adoptive cell therapies. In some embodiments, the immune cells are primary immune cells. In some embodiments, the immune cells are lymphocytes, such as T and B cells. In some embodiments, the immune cells are natural killer (NK) cells. In some embodiments, the immune cells are a mixture of lymphocytes and NK cells. In some embodiments, the immune cells are obtained from the peripheral blood of a donor subject. In some embodiments, the immune cells are peripheral blood mononuclear cells (PBMC). In some embodiments, the immune cells are obtained from the spleen or lymph nodes of a donor subject.

In some embodiments, the immune cells are modified following isolation from a donor. For example, in some embodiments, the immune cells are CAR T cells.

In some embodiments, the immune cells are obtained from a subject that has a chronic infection. In some embodiments, the chronic infection is a bacterial infection, a viral infection, a fungal infection, a protozoan infection, or parasitic infection.

In some embodiments, the immune cells are obtained from a subject that has a tumor. In some embodiments, the immune cells are T cells that have infiltrated a tumor (e.g., tumor infiltrating lymphocytes). In some embodiments, the T cells are removed during surgery of a tumor. For example, in some embodiments, the T cells are isolated after removal of tumor tissue by biopsy.

In some embodiments, the T cells are isolated from a sample containing a population of cells, such as a blood, lymph, spleen or tissue biopsy sample. T cells can be isolated from a population of cells by any means known in the art. In one embodiment, the methods comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells can include, but are not limited to, any one or more of mechanically dissociating (e.g., mincing) the tumor, enzymatically dissociating (e.g., digesting) the tumor, and/or aspiration (e.g., as with a needle).

The bulk population of T cells obtained from a tumor sample can comprise any suitable type of T cell. Preferably, the bulk population of T cells obtained from a tumor sample comprises tumor infiltrating lymphocytes (TILs).

The population of immune cells can be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals can be non-human primates, e.g., of the order Primates, Ceboids, or Simoids (monkeys) or of the order Anthropoids (humans and apes). In some embodiments, the mammal can be a mammal of the order Rodentia, such as mice and hamsters. Preferably, the mammal is a non-human primate or a human. An exemplary mammal is a human.

In some embodiments, the subject to receive the immune cells is also the donor of the immune cells (i.e., autologous ACT). In some embodiments, the subject to receive the immune cells is different than the donor of the tumor sample (i.e. allogenic ACT).

T cells can be obtained from a number of sources, including peripheral blood mononuclear cells, bone marrow, lymph node tissue, spleen tissue, and tumors. In certain embodiments, T cells can be obtained from a unit of blood collected from a subject using any number of techniques known to the skilled artisan, such as Ficoll separation. In one embodiment, cells from the circulating blood of an individual are obtained by apheresis or leukopheresis. The apheresis product typically contains lymphocytes, including T cells, monocytes, granulocytes, B cells, other nucleated white blood cells, red blood cells, and platelets. In one embodiment, the cells collected by apheresis can be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and can lack magnesium or can lack many if not all divalent cations. Initial activation steps in the absence of calcium lead to magnified activation. As those of ordinary skill in the art would readily appreciate, a washing step can be accomplished by methods known to those in the art, such as by using a semi-automated “flow-through” centrifuge (for example, the Cobe 2991 cell processor) according to the manufacturer's instructions. After washing, the cells can be resuspended in a variety of biocompatible buffers, such as, for example, Ca-free, Mg-free PBS. Alternatively, the undesirable components of the apheresis sample can be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. In one embodiment, the incubation time period is 24 hours. For isolation of T cells from patients with leukemia, use of longer incubation times, such as 24 hours, can increase cell yield. Longer incubation times can be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. In one embodiment, the method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) can be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology can be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it can be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that can weakly express target antigens of interest, such as CD28-negative T cells, or from samples where there are many tumor cells present (e.g., leukemic blood, tumor tissue). Such populations of cells can have therapeutic value and would be desirable to obtain. For example, using high concentration of cells allows more efficient selection of CD8+ T cells that normally have weaker CD28 expression.

In a related embodiment, it can be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/mL. In other embodiments, the concentration used can be from about 1×10⁵/ml to 1×10⁶/mL, and any integer value in between. Thus, the concentration used may be from about 1×10⁵/mL, about 1.1×10⁵/mL, about 1.2×10⁵/mL, about 1.3×10⁵/mL, about 1.4×10⁵/mL, about 1.5×10⁵/mL, about 1.6×10⁵/mL, about 1.7×10⁵/mL, about 1.8×10⁵/mL, about 1.9×10⁵/mL, about 2×10⁵/mL, about 2.2×10⁵/mL, about 2.4×10⁵/mL, about 2.6×10⁵/mL, about 2.8×10⁵/mL, about 3×10⁵/mL, about 3.2×10⁵/mL, about 3.4×10⁵/mL, about 3.6×10⁵/mL, about 3.8×10⁵/mL, about 4×10⁵/mL, about 4.2×10⁵/mL, about 4.4×10⁵/mL, about 4.6×10⁵/mL, about 4.8×10⁵/mL, about 5×10⁵/mL, about 5.5×10⁵/mL, about 6×10⁵/mL, about 6.5×10⁵/mL, about 7×10⁵/mL, about 7.5×10⁵/mL, about 8×10⁵/mL, about 8.5×10⁵/mL, about 9×10⁵/mL, about 9.5×10⁵/mL, about 1×10⁶/mL, or any integer value in between.

T cells can also be frozen. The freeze and subsequent thaw step can provide a more uniform product by removing granulocytes and to some extent monocytes in the cell population. After a washing step to remove plasma and platelets, the cells can be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing can be used as well as uncontrolled freezing immediately at −20° C. or in liquid nitrogen.

In some embodiments, cells are directly labeled with an epitope-specific reagent for isolation and/or enrichment by flow cytometry followed by characterization of cell phenotypes. In some embodiments, immune cells are isolated by contacting the immune cell specific antibodies. Sorting of any cells of the present invention, can be carried out using any of a variety of commercially available cell sorters, including, but not limited to, MoFlo sorter (DakoCytomation, Fort Collins, Colo.), FACSAria™, FACSArray™, FACSVantage™ BD™ LSR II, and FACSCalibur™ (BD Biosciences, San Jose, Calif.).

In one embodiment, the method further comprises expanding the numbers of T cells in the enriched cell population. Such methods are described in U.S. Pat. No. 8,637,307 and is hereby incorporated by reference in its entirety. The T cells can be expanded before or after the cells are contacted with the agent that disrupts CK1G2 activity or the nucleic acid encoding the mutant PKC theta protein. The numbers of T cells can be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000-fold, or most preferably at least about 100,000-fold. The numbers of T cells can be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells can be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal can be used in soluble form. Ligands can be attached to the surface of a cell, to an Engineered Multivalent Signaling Platform (EMSP), or immobilized on a surface. In a one embodiment both primary and secondary agents are co-immobilized on a surface, for example a bead or a cell. In one embodiment, the molecule providing the primary activation signal can be a CD3 ligand, and the co-stimulatory molecule can be a CD28 ligand or 4-1BB ligand. In some embodiments, the cells are expanded by stimulation with one or more antigens, such as a melanoma tumor antigen or antigens derived from the patient's tumor.

In some embodiments, the isolated immune cells are immediately contacted with the agent that disrupts CK1G2 activity or the nucleic acid encoding the mutant PKC theta protein following isolation. In other embodiments, the isolated immune cells are stored in a suitable buffer and frozen prior to being contacted with the agent that disrupts CK1G2 activity or the nucleic acid encoding the mutant PKC theta protein. In some embodiments, the isolated immune cells are immediately contacted with the agent that disrupts CK1G2 activity or the nucleic acid encoding the mutant PKC theta protein following isolation and the treated cells are stored in a suitable buffer and frozen until needed for administration to the patient.

In certain embodiments, the isolated immune cells (e.g., a mixed population immune cells or isolated types, such as CD8+ T cells) are contacted with the agent that disrupts CK1G2 activity or the nucleic acid encoding the mutant PKC theta protein for a period of time sufficient to be taken up by the cells. In some embodiments, the agent that disrupts CK1G2 activity, or a nucleic acid encoding the same, or the nucleic acid encoding the mutant PKC theta protein are contained in a composition or vector. In some embodiments, the immune cells are contacted with a composition or vector containing the agent that disrupts CK1G2 activity, or a nucleic acid encoding the same, or the nucleic acid encoding the mutant PKC theta protein for less than about 24 hours, less than about 23 hours, less than about 22 hours, less than about 21 hours, less than about 20 hours, less than about 19 hours, less than about 18 hours, less than about 17 hours, less than about 16 hours, less than about 15 hours, less than about 14 hours, less than about 13 hours, less than about 12 hours, less than about 11 hours, less than about 10 hours, less than about 9 hours, less than about 8 hours, less than about 7 hours, less than about 6 hours, less than about 5 hours, less than about 4 hours, less than about 3 hours, less than about 2 hours, or less than about 1 hour.

In certain embodiments, the immune cells are contacted with an agent that disrupts CK1G2 activity or a nucleic acid encoding the mutant PKC theta protein for less than about 55 minutes, less than about 50 minutes, less than about 45 minutes, less than about 40 minutes, less than about 35 minutes, less than about 30 minutes, less than about 29 minutes, less than about 28 minutes, less than about 27 minutes, less than about 26 minutes, less than about 25 minutes, less than about 24 minutes, less than about 23 minutes, less than about 22 minutes, less than about 21 minutes, less than about 20 minutes, less than about 19 minutes, less than about 18 minutes, less than about 17 minutes, less than about 16 minutes, less than about 15 minutes, less than about 14 minutes, less than about 13 minutes, less than about 12 minutes, less than about 11 minutes, or less than about 10 minutes. In certain embodiments, the immune cells are contacted with the agent that disrupts CK1G2 activity or the nucleic acid encoding the mutant PKC theta protein for about 1 hour. In some embodiments, the agent that disrupts CK1G2 activity or the nucleic acid encoding the mutant PKC theta protein are contained in a composition or vector.

In certain embodiments, the immune cells are contacted with the agent that disrupts CK1G2 activity or the nucleic acid encoding the mutant PKC theta protein for 24 hours or longer. In certain embodiments, the immune cells are contacted with a composition or vector comprising the agent that disrupts CK1G2 activity, or a nucleic acid encoding the same, or the nucleic acid encoding the mutant PKC theta protein for less than about 12 days, less than about 11 days, less than about 10 days, less than about 9 days, less than about 8 days, less than about 7 days, less than about 6 days, less than about 5 days, less than about 4 days, less than about 2 days, or less than about 1 day. In some embodiments, the agent that disrupts CK1G2 activity, or a nucleic acid encoding the same, or the nucleic acid encoding the mutant PKC theta protein are contained in a composition or vector.

In certain embodiments that may be combined with any of the preceding embodiments, the cells are contacted with a composition comprising the agent that disrupts CK1G2 activity or the nucleic acid encoding the mutant PKC theta protein a concentration of 0.5 μg/ml to 500 μg/ml. In some embodiments, the concentration is at least 0.5 μg/ml, at least 0.6 μg/ml, at least 0.7 μg/ml, at least 0.8 μg/ml, at least 0.9 μg/ml, at least 1 μg/ml, at least 2 μg/ml, at least 3 μg/ml, at least 4 μg/ml, at least 5 μg/ml, at least 6 μg/ml, at least 7 μg/ml, at least 8 μg/ml, at least 9 μg/ml, at least 10 μg/ml, at least 15 μg/ml, at least 20 μg/ml, at least 25 μg/ml, at least 30 μg/ml, at least 35 μg/ml, at least 40 μg/ml, at least 45 μg/ml, at least 50 μg/ml, at least 55 μg/ml, at least 60 μg/ml, at least 65 μg/ml, at least 70 μg/ml, at least 75 μg/ml, at least 80 μg/ml, at least 85 μg/ml, at least 90 μg/ml, at least 95 μg/ml, at least 100 μg/ml, at least 110 μg/ml, at least 120 μg/ml, at least 130 μg/ml, at least 140 μg/ml, at least 150 μg/ml, at least 160 μg/ml, at least 170 μg/ml, at least 180 μg/ml, at least 190 μg/ml, at least 200 μg/ml, at least 220 μg/ml, at least 240 μg/ml, at least 260 μg/ml, at least 280 μg/ml, at least 300 μg/ml, at least 320 μg/ml, at least 340 μg/ml, at least 360 μg/ml, at least 380 μg/ml, at least 400 μg/ml, at least 420 μg/ml, at least 440 μg/ml, at least 460 μg/ml, at least 480 μg/ml, at least 500 μg/ml. In certain embodiments that may be combined with any of the preceding embodiments, the cells are contacted with a composition comprising a vector comprising the the agent that disrupts CK1G2 activity, or the nucleic acid encoding the same, or the nucleic acid encoding the mutant PKC theta protein, wherein the vector is at a concentration of 0.5 μg/ml to 500 μg/ml. In some embodiments, the concentration is at least 0.5 μg/ml, at least 0.6 μg/ml, at least 0.7 μg/ml, at least 0.8 μg/ml, at least 0.9 μg/ml, at least 1 μg/ml, at least 2 μg/ml, at least 3 μg/ml, at least 4 μg/ml, at least 5 μg/ml, at least 6 μg/ml, at least 7 μg/ml, at least 8 μg/ml, at least 9 μg/ml, at least 10 μg/ml, at least 15 μg/ml, at least 20 μg/ml, at least 25 μg/ml, at least 30 μg/ml, at least 35 μg/ml, at least 40 μg/ml, at least 45 μg/ml, at least 50 μg/ml, at least 55 μg/ml, at least 60 μg/ml, at least 65 μg/ml, at least 70 μg/ml, at least 75 μg/ml, at least 80 μg/ml, at least 85 μg/ml, at least 90 μg/ml, at least 95 μg/ml, at least 100 μg/ml, at least 110 μg/ml, at least 120 μg/ml, at least 130 μg/ml, at least 140 μg/ml, at least 150 μg/ml, at least 160 μg/ml, at least 170 μg/ml, at least 180 μg/ml, at least 190 μg/ml, at least 200 μg/ml, at least 220 μg/ml, at least 240 μg/ml, at least 260 μg/ml, at least 280 μg/ml, at least 300 μg/ml, at least 320 μg/ml, at least 340 μg/ml, at least 360 μg/ml, at least 380 μg/ml, at least 400 μg/ml, at least 420 μg/ml, at least 440 μg/ml, at least 460 μg/ml, at least 480 μg/ml, at least 500 μg/ml.

In some embodiments, the immune cells that are contacted with an agent that disrupts CK1G2 activity or a nucleic acid encoding the mutant PKC theta protein are T cells with genetically modified antigen receptors, including chimeric antigen receptor (CAR)-T cells and T cells comprising antiviral or anticancer TCRs. Various strategies can, for example, be employed to genetically modify T cells by altering the specificity of the T cell receptor (TCR), for example, by introducing new TCR a and R chains with selected peptide specificity (see, e.g., U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379). Chimeric antigen receptors (CARs) can be used in order to generate immunoresponsive cells, such as T cells, specific for selected targets, such as malignant cells, with a wide variety of receptor chimera constructs having been described (see, e.g., U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Methods for the preparation of CAR T cells are known in the art and can be used in combination with the methods provided herein to generate modified CAR T cells comprising disrupted CK1G2 activity or a mutant PKC theta protein as described herein. In some embodiments, disrupted CK1G2 activity or a mutant PKC theta polypeptide can improve the expansion of the CAR T cells prior to administration to the subject or after administration to the subject.

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

In some embodiments, the T cells expressing a desired CAR are selected through co-culture with γ-irradiated activating and propagating cells (AaPC), which co-express the antigen and co-stimulatory molecules. In some embodiments, the engineered CAR T-cells are expanded, for example by co-culture on AaPC in presence of soluble factors, such as IL-2 and IL-21. This expansion can for example be carried out so as to provide memory CAR+ T cells. In this way, CAR T cells can be provided that have specific cytotoxic activity against antigen-bearing cells (optionally in conjunction with production of desired chemokines such as interferon-γ).

In some embodiments, the CAR T-cells are contacted with an agent that disrupts CK1G2 activity or a nucleic acid encoding a mutant PKC theta protein provided herein in vitro to generation a modified CAR T cells for the treatment of a disease or condition associated with T cell exhaustion or T cell impairment (e.g., a chronic infection or cancer). The modified CAR T cells can be administered according to any suitable method, including the methods for administration of the modified immune cells as described above.

### V. Mutant PKC Theta and CK1G2 Disruption Agent Expression Constructs

The entire disclosure and any embodiments provided herein are also applicable to expression constructs encoding a PKC theta overexpression construct, wherein the PKC theta is a wild type PKC theta lacking mutations (e.g., PKC theta encoded by SEQ ID NO: 3 or having the polypeptide sequence of SEQ ID NO: 4). In some embodiments, the nucleic acid sequence encoding the agent that disrupts CK1G2 activity or the nucleic acid sequence encoding the PKC mutant theta protein comprises one or more additional regulatory elements. In some embodiments the one or more additional regulatory elements are promoters, enhancers, internal ribosomal entry sites (IRES), 2A sequence elements (e.g., T2A or P2A), and other expression control elements (e.g., transcription termination signals, such as polyadenylation signals and poly-U sequences). One of ordinary skill in the art will be familiar with such regulatory elements and their use to drive expression of a nucleic acid sequence encoding the agent that disrupts CK1G2 activity or the mutant PKC theta protein in a desired host cell.

Integrative or self-replicative vectors can be used for the purpose of introducing a CK1G2 disruption agent or a mutant PKC theta expression cassette into an immune cell of choice. In an expression cassette, the coding sequence for the CK1G2 disruption agent or mutant PKC theta polypeptide is operably linked to a promoter, such as an inducible promoter or a constitutive promoter. Inducible promoters are promoters that initiate increased levels of transcription from DNA under their control in response to some change in culture conditions, e.g., the presence or absence of a nutrient or a change in temperature. In some embodiments, the nucleic acid encoding the CK1G2 disruption agent or the mutant PKC theta protein is codon optimized for mammalian expression.

Exemplary promoters that are recognized by a variety of potential host cells are well known. These promoters can be operably linked to the CK1G2 disruption agent or mutant PKC theta polypeptide-encoding DNA by removing the promoter from the source DNA, if present, by restriction enzyme digestion and inserting the isolated promoter sequence into the vector. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the 3-actin promoter, the phosphoglycerol kinase (PGK) promoter, and the EF1α promoter.

### VI. Administration of the Modified Immune Cells to a Subject

As provided previously, the present disclosure provides a method for treating T cell exhaustion, or T cell impairment, in a subject in need thereof, wherein the method comprises administering an effective amount of one or more modified immune cells to the subject, wherein the one or more modified immune cells comprise disrupted CK1G2 activity or a PKC theta mutant protein and/or a nucleic acid encoding a PKC theta mutant protein. Additionally, the entire disclosure and any embodiments provided herein is also applicable to one or more modified immune cells comprising a PKC theta overexpression construct, wherein the PKC theta is a wild type PKC theta lacking mutations (e.g., PKC theta encoded by SEQ ID NO: 3 or having the polypeptide sequence of SEQ ID NO: 4). In some embodiments, the methods comprise an adoptive cell therapy (ACT) method. In some embodiments, the one or more modified immune cells further comprises a chimeric antigen receptor (CAR).

Any method known to those in the art for contacting a cell, organ or tissue with one or more modified immune cells disclosed herein may be employed. Suitable methods include in vitro, ex vivo, or in vivo methods. In vivo methods typically include the administration of one or more modified immune cells to a mammal, suitably a human. The one or more modified immune cells described herein are administered to the subject in effective amounts (i.e., amounts that have desired therapeutic effect). The dose and dosage regimen will depend upon the degree of the disease state of the subject, the characteristics of the particular modified immune cell used, e.g., its therapeutic index, and the subject's history.

The effective amount may be determined during pre-clinical trials and clinical trials by methods familiar to physicians and clinicians. An effective amount of one or more modified immune cells useful in the methods disclosed herein may be administered to a mammal in need thereof by any of a number of well-known methods for administering pharmaceutical compounds and cells. The modified immune cells may be administered systemically or locally.

For the administration of the modified immune cell compositions, any suitable method for administration of cells to a subject may be employed. For example, suitable methods for adoptive cell therapy typically involve systemic infusion (e.g., intravenous or intraperitoneal infusion) of the modified immune cells together with a pharmaceutically acceptable carrier, diluent or excipient. In some embodiments, the modified immune cells are administered locally, for example, intratumorally or at the site of infection.

In some embodiments, the one or more modified immune cells described herein can be incorporated into pharmaceutical compositions for administration, singly or in combination, to a subject for the treatment or prevention of immune cell exhaustion. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions of the present disclosure can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of modified immune cells described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compositions may be within a range of circulating concentrations that include the ED50 with little or no toxicity. The dosage may vary within this range depending upon the dosage form employed and the route of administration utilized. For any compositions used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more modified immune cells disclosed herein sufficient for achieving a therapeutic or prophylactic effect, range from about 0.1 million cells/kg body weight to about 5 million cells/kg body weight. In some embodiments, the effective amount of the one or more modified immune cells is about 0.1 million cells/kg body weight. In some embodiments, the effective amount of the one or more modified immune cells is about 0.5 million cells/kg body weight. In some embodiments, the effective amount of the one or more modified immune cells is about 1.0 million cells/kg body weight. In some embodiments, the effective amount of the one or more modified immune cells is about 1.5 million cells/kg body weight. In some embodiments, the effective amount of the one or more modified immune cells is about 2.0 million cells/kg body weight. In some embodiments, the effective amount of the one or more modified immune cells is about 2.5 million cells/kg body weight. In some embodiments, the effective amount of the one or more modified immune cells is about 3.0 million cells/kg body weight. In some embodiments, the effective amount of the one or more modified immune cells is about 3.5 million cells/kg body weight. In some embodiments, the effective amount of the one or more modified immune cells is about 4.0 million cells/kg body weight. In some embodiments, the effective amount of the one or more modified immune cells is about 4.5 million cells/kg body weight. In some embodiments, the effective amount of the one or more modified immune cells is about 5.0 million cells/kg body weight. In terms of total cells administered, the effective amount of the one or more modified immune cells disclosed herein sufficient for achieving a therapeutic or prophylactic effect, can range from about 10 million cells to about 600 million cells. In some embodiments, the effective amount of the one or more modified immune cells is about 10 million cells. In some embodiments, the effective amount of the one or more modified immune cells is about 10 million cells to about 250 million cells. In some embodiments, the effective amount of the one or more modified immune cells is about 60 million cells to about 600 million cells. In some embodiments, the effective amount of the one or more modified immune cells is about 50 million cells to about 110 million cells. In some embodiments, the effective amount of the one or more modified immune cells is about 300 million cells to about 460 million cells. In some embodiments, the effective amount of the one or more modified immune cells is about 10 million cells. In some embodiments, the effective amount of the one or more modified immune cells is about 50 million cells. In some embodiments, the effective amount of the one or more modified immune cells is about 100 million cells. In some embodiments, the effective amount of the one or more modified immune cells is about 250 million cells. In some embodiments, the effective amount of the one or more modified immune cells is about 300 million cells. In some embodiments, the effective amount of the one or more modified immune cells is about 350 million cells. In some embodiments, the effective amount of the one or more modified immune cells is about 400 million cells. In some embodiments, the effective amount of the one or more modified immune cells is about 450 million cells. In some embodiments, the effective amount of the one or more modified immune cells is about 500 million cells. In some embodiments, the effective amount of the one or more modified immune cells is about 550 million cells. In some embodiments, the effective amount of the one or more modified immune cells is about 600 million cells.

An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### VII. Combination Therapies

In some embodiments, the one or more modified immune cells comprising disrupted CK1G2 activity or a PKC theta mutant protein and/or a nucleic acid encoding a PKC theta mutant protein are administered with an additional therapeutic agent. The entire disclosure and any embodiments provided herein are also applicable to one or more modified immune cells comprising a PKC theta overexpression construct, wherein the PKC theta is a wild type PKC theta lacking mutations (e.g., PKC theta encoded by SEQ ID NO: 3 or having the polypeptide sequence of SEQ ID NO: 4). Suitable therapeutic agents for combination therapy can be selected based on the condition or disease to be treated. In some embodiments, additional therapeutic agent is administered prior to, simultaneously with, intermittently with, or following treatment with the modified immune cells. In some embodiments, the additional therapeutic agent is an immunomodulator, such as an interleukin (e.g. IL-2, IL-7, IL-12), a cytokine, a chemokine, or and immunomodulatory drug. In some embodiments, the additional therapeutic agent is an anticancer agent (e.g., chemotherapy, radiation therapy, oncolytic agent, immunotherapy, monoclonal antibodies, anti-cancer nucleic acids or proteins, anti-cancer viruses or microorganisms, and any combinations thereof), an antibacterial agent or an antiviral agent. In some embodiments, the additional therapeutic agent comprises an anti-PD-1 inhibitor, an anti-PD-L1 inhibitor, an anti-IL-10 inhibitor, an anti-LAG-3 inhibitor, an anti-CTLA-4 inhibitor, an anti-TIM3 inhibitor, an anti-IL-10R inhibitor, IL-2, IL-21, an engineered cytokine (e.g., H9T or DR-18), and any combination thereof.

### VIII. Kits

Kits according to this embodiment can comprise a carrier means, such as a box, carton, tube, having in close confinement therein one or more containers, such as vials, tubes, ampoules, bottles, syringes, or bags. The kits can also comprise associated instructions for using or generating the modified immune cells comprising disrupted CK1G2 activity or a PKC theta mutant protein and/or a nucleic acid encoding a PKC theta mutant protein of the present technology. Additionally or alternatively, the kits may comprise expression constructs encoding a PKC theta overexpression construct, wherein the PKC theta is a wild type PKC theta lacking mutations (e.g., PKC theta encoded by SEQ ID NO: 3 or having the polypeptide sequence of SEQ ID NO: 4), and/or one or more modified immune cells comprising the PKC theta overexpression construct. In some embodiments, the kit comprises an effective amount of an adoptive cell therapy, such as modified immune cells comprising disrupted CK1G2 activity or a PKC theta mutant protein and/or a nucleic acid encoding a PKC theta mutant protein. In some embodiments, the kit comprises one for more reagents for the detection of the administered modified immune cells. In some embodiments, the kit comprises a nucleic acid encoding a PKC theta mutant protein. In some embodiments, the kit comprises an agent for disrupting CK1G2 activity, or a nucleic acid encoding the same.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Materials and Methods

Most of the reagents used in these experiments, their source, and identifier are indicated in Table A:

TABLE A

| | | |
|---|---|---|
| REAGENT or | | |
| RESOURCE | SOURCE | IDENTIFIER |

Antibodies

| | | |
|---|---|---|
| Anti-CD8a BUV395 | BD Biosciences | Clone 53-6.7, cat #563786 |
| Anti-CD8a BV421 | BioLegend | Clone 53-6.7, cat #100738 |
| Anti-CD90.1 BUV737 | BD Biosciences | Clone OX-7, cat #612837 |
| Anti-CD90.1 BD | BD Biosciences | Clone OX-7, cat #741110 |
| OptiBuild ™ BUV496 | | |
| Anti-CD45.1 APC | BioLegend | Clone A20, cat #110714 |
| Anti-CD45.2 BD | BD Biosciences | Clone 104, cat #564616 |
| Horizon ™ BUV395 | | |
| Anti-CX3CR1 | BioLegend | Clone SA011F11, Cat #149040 |
| APC/Fire ™ 750 | | |
| PKCθ (unconjugated) | Cell Signaling | Clone E1I7Y, cat #13643S |
| | Technology | |
| Anti-GFP Alexa Fluor | BioLegend | Clone FM264G, Cat #338008 |
| 488 | | |
| Anti-GFP Alexa Fluor | Invitrogen | Polyclonal, Cat #A-21311 |
| 488 | | |
| Anti-CD101 PE/Cy7 | Invitrogen | Clone Moushi101, cat #25-1011-82 |
| Anti-CD101 APC | Invitrogen | Clone Moushi101, cat #17-1011-82 |
| Anti-rabbit IgG (H + L), | Cell Signaling | Polyclonal, Cat #4414S |
| F(ab′)₂ Fragment Alexa | Technology | |
| Fluor 647 | | |
| Anti-rabbit IgG (H + L), | Cell Signaling | Polyclonal, Cat #4412S |
| F(ab′)₂ Fragment Alexa | Technology | |
| Fluor 488 | | |
| Anti-TOX PE | Invitrogen | Clone TXRX10, cat #12-6502-82 |
| Anti-PD-1 BV785 | BioLegend | Clone 29F.1A12, cat # 135225 |
| Anti-CD45 BV711 | BioLegend | Clone 30-F11, cat #103147 |
| Anti-SLAMF6 (Ly108) | BD Biosciences | Clone 13G3, cat # 745250 |
| BD OptiBuild ™ BV605 | | |
| Anti-TIM-3 BV421 | BioLegend | Clone RMT3-23, cat # 119723 |
| Anti-TIM-3 BV711 | BioLegend | Clone RMT3-23, cat # 119727 |
| Anti-TIM-3 PE/Cy7 | BioLegend | Clone RMT3-23, cat # 119716 |
| Anti-Granzyme B APC | BioLegend | Clone QA16A02, cat #372204 |
| Anti-Granzyme B | BioLegend | Clone QA16A02, cat #372210 |
| APC/Fire ™ 750 | | |
| Anti-CXCR6 PE | BioLegend | Clone SA051D1, cat # 151104 |
| Anti-CXCR6 PE/Cy7 | BioLegend | Clone SA051D1, cat # 151119 |
| Anti-CXCR6 BV421 | BioLegend | Clone SA051D1, cat #151109 |
| Anti-KLRG1 PE | BioLegend | Clone 2F1/KLRG1, cat #138408 |
| Anti-KLRG1 PE/Cy7 | BioLegend | Clone 2F1/KLRG1, cat #138416 |
| Anti-TNF APC | BioLegend | Clone MP6-XT22, cat #506308 |
| Anti-TNF FITC | BioLegend | Clone MP6-XT22, cat #506304 |
| Anti-IL-2 PE | BioLegend | Clone JES6-5H4, cat #503808 |
| Anti-IFNγ BV421 | BioLegend | Clone XMG1.2, cat #505830 |
| Anti-T-bet BV421 | BioLegend | Clone 4B10, cat #644816 |
| Anti-PKC eta | Abcam | Clone EPR18513, cat #ab179524 |
| (unconjugated) | | |
| Anti-Ki-67 BV421 | BioLegend | Clone 16A8, cat #652411 |
| Anti-CD25 FITC | BioLegend | Clone PC61, cat #102006 |
| Anti-CD69 FITC | BioLegend | Clone H1.2F3, cat #104506 |
| Anti-Phospho p38 MAPK | Cell Signaling | Clone D3F9, cat #4511S |
| (Thr180/Tyr182) | Technology | |
| Anti-Phospho p44/42 | Cell Signaling | Clone D13.14.4E, cat #4370 |
| MAPK Erk1/2 | Technology | |
| (Thr202/Tyr204) | | |
| Anti-Phospho SAPK/JNK | Cell Signaling | Clone 81E11, cat #4668 |
| (Thr183/Tyr185) | Technology | |
| Anti-Phospho ATF2 | Cell Signaling | Clone A8J7P, cat #15411 |
| (Thr71) / ATF7 (Thr53) | Technology | |
| Anti-Phospho S6 | Cell Signaling | Clone D57.2.2E, cat #4858 |
| (Ser235/Ser236) | Technology | |
| Anti-Phospho c-Fos | Cell Signaling | Clone D82C12, cat #5348 |
| (Ser32) | Technology | |
| Anti-Phospho c-Jun | Cell Signaling | Clone D47G9, cat #3270 |
| (Ser73) | Technology | |
| Anti-Phospho NF-κB p65 | Cell Signaling | Clone 93H1, cat #3033 |
| (Ser536) | Technology | |
| Anti-cofilin | Cell Signaling | Clone D3F9, cat #5175 |
| | Technology | |
| Anti-Histone H3 | Cell Signaling | Clone D1H2, cat #4499 |
| | Technology | |
| Anti-CK1 gamma-2 | Invitrogen | Polyclonal, PA5-97637 |
| Anti-Pellino1 | Cell Signaling | Clone D2Z4F, cat #31474 |
| | Technology | |
| Anti-CARD11 | Cell Signaling | Clone 1D12, cat #4435 |
| (CARMA1) | Technology | |
| Anti-Phospho Nur77 | Cell Signaling | Clone D22G5, cat #5095 |
| (Ser351) | Technology | |
| Goat anti-Rabbit IgG | Invitrogen | 31460 |
| (H + L) Secondary | | |
| Antibody, HRP | | |
| Goat anti-Mouse IgG | Tonbo Biosciences | 72-8042-M001 |
| (H&L) - Affinity Pure, | | |
| HRP conjugate | | |
| Anti-CD3e (Purified | BD Biosciences | Clone 145-2C11, cat #553057 |
| NA/LE) | | |
| AffiniPure Goat Anti- | Jackson | 127-005-099 |
| Armenian Hamster IgG | ImmunoResearch | |
| (H + L) | | |
| Anti-CD28 (Purified) | BD Biosciences | Clone 37.51, cat #553295 |
| Anti-beta actin | Proteintech | 66009-1-Ig |
| LIVE/DEAD ™ Fixable | Invitrogen | Cat #L34972 |
| Red Dead Cell Stain | | |
| Ghost Dye ™ Red 780 | Tonbo Biosciences | Cat #13-0865-T500 |

Chemicals, peptides, and recombinant proteins

| | | |
|---|---|---|
| X-tremeGENE 9 | MilliporeSigma | 6365779001 / XTG9-RO |
| Polybrene | MilliporeSigma | TR-1003 |
| IL-2 (recombinant, | PeproTech | 212-12 |
| murine) | | |
| Penicillin-Streptomycin | Gibco / ThermoFisher | 15140122 |
| Geneticin | | |
| D4476 | MedChem Express | HY-10324 |
| AZD1208 | Selleck Chemicals | S7104 |
| | LLC | |
| WNK-IN-11 | MedChem Express | HY-112094 |
| Compound 20 | Selleck Chemicals | S6577 |
| | LLC | |
| GSK-626616 | MedChem Express | HY-105309 |
| Ralimetinib dimesylate | Selleck Chemicals | S1494 |
| | LLC | |
| Phorbol 12-myristate 13- | Millipore Sigma | P1585 |
| acetate (PMA) | | |
| ACK buffer | ThermoFisher | RGF3015 |
| | Scientific | |
| Collagenase type IV | Millipore Sigma | C5138 |
| DNAseI | Millipore Sigma / | 10104159001 |
| | Roche | |
| S.p. Cas9 Nuclease V3 | Integrated DNA | 1081059 |
| | Technologies | |
| RIPA Lysis and | ThermoFisher | 89900 |
| Extraction buffer | Scientific | |
| Trypsin-EDTA (0.25%), | ThermoFisher | 25-200-056 |
| phenol red | Scientific | |
| cOmplete Protease | Millipore Sigma / | 11697498001 |
| Inhibitor Cocktail | Roche | |
| PhosSTOP | Millipore Sigma / | 4906845001 |
| | Roche | |
| Laemmli sample buffer, | Bio-Rad | 1610747 |
| 4x | | |
| 2-Mercaptoethanol | Millipore Sigma | M6250 |
| Phorbol 12-myristate 13- | Millipore Sigma | P1585 |
| acetate (PMA) | | |
| Dimethyl Sulfoxide | Corning | 25-950-CQC |
| (DMSO) | | |
| Ethanol | Fisher Scientific | BP2818 |
| Glycerol | Fisher Scientific | BP229-1 |

Kits and critical commercial assays

| | | |
|---|---|---|
| P3 Primary Cell 4D- | Lonza | V4XP-3032 |
| Nucleofector X Kit | | |
| 4-15% Mini-PROTEAN | Bio-Rad | 4561086 |
| TGX Precast Protein Gels | | |
| SuperSignal ™ West Pico | ThermoFisher | 34580 |
| PLUS Chemiluminescent | Scientific | |
| Substrate | | |
| Fe-NTA Phosphopeptide | ThermoFisher | A32992 |
| Enrichment Kit | Scientific | |
| High pH Reversed-Phase | ThermoFisher | 84868 |
| Peptide Fractionation Kit | Scientific / Pierce | |

Experimental models: Organisms/strains

| | | |
|---|---|---|
| Mouse: C57BL/6J | The Jackson | RRID: IMSR_JAX:000664 |
| | Laboratory | |
| Mouse: P14 | The Jackson | RRID: MMRRC_037394-JAX |
| | Laboratory | |
| Mouse: Thy1.1 | The Jackson | RRID: IMSR_JAX:000406 |
| | Laboratory | |
| Mouse: C57BL/6J | The Jackson | RRID: IMSR_JAX:002014 |
| CD45.1 | Laboratory | |

Plasmids

| | | |
|---|---|---|
| MigR1² | Addgene | 27490 |
| MSCV-IRES-Thy1.1 | Addgene | 17442 |
| DEST³ | | |
| pTM 194: MSCV- | See Experimental | N/A |
| Thy1.1-Prkcq | Examples | |
| pTM 210: MigR1-Prkcq | See Experimental | N/A |
| (residues 327-707) | Examples | |
| pTM 211: MigR1-Prkcq | See Experimental | N/A |
| (residues 376-707) | Examples | |
| pTM 212: MigR1-Prkch | See Experimental | N/A |
| (residues 299-683) | Examples | |
| pTM 213: MigR1-Prkch | See Experimental | N/A |
| (residues 359-683) | Examples | |
| pTM 226: MSCV-Thy1.1 | See Experimental | N/A |
| Prkcq (K376R, K384R, | Examples | |
| K388R, K393R, K400R) | | |
| pTM 227: MSCV-Thy1.1 | See Experimental | N/A |
| Prkcq (K409R, K412R, | Examples | |
| K413R, K429R, K451R) | | |
| pTM 228: MSCV-Thy1.1 | See Experimental | N/A |
| Prkcq (K475R, K498R, | Examples | |
| K506R, K519R, K527R) | | |
| pTM 229: MSCV-Thy1.1 | See Experimental | N/A |
| Prkcq (K535R, K555R, | Examples | |
| K607R, K612R) | | |
| pTM 230: MSCV-Thy1.1 | See Experimental | N/A |
| Prkcq (K620R, K645R, | Examples | |
| K654R, K656R, K666R, | | |
| K672R) | | |
| pTM 240: MSCV-Thy1.1 | See Experimental | N/A |
| Prkcq (K409R) | Examples | |
| pTM 241: MSCV-Thy1.1 | See Experimental | N/A |
| Prkcq (K412R) | Examples | |
| pTM 242: MSCV-Thy1.1 | See Experimental | N/A |
| Prkcq (K413R) | Examples | |
| pTM 243: MSCV-Thy1.1 | See Experimental | N/A |
| Prkcq (K429R) | Examples | |
| pTM 244: MSCV-Thy1.1 | See Experimental | N/A |
| Prkcq (K451R) | Examples | |
| pTM 261: MigR1-Prkcq | See Experimental | N/A |
| | Examples | |
| pTM 265: MigR1-Prkcq | See Experimental | N/A |
| (K413R) | Examples | |

Software and algorithms

| | | |
|---|---|---|
| FlowJo | FlowJo, LLC | https://www.flowjo.com/ |
| R 3.6 | CRAN | |
| RStudio (version) | Rstudio | |
| PEAKS | Bioinformatics | https://www.bioinfor.com/peaks- |
| | Solutions Inc. | studio/ |
| DIA-NN | Demichev et al. 2020⁴ | https://github.com/vdemichev/DiaNN |
| MaxLFQ | Cox et al. 2014⁵ | |
| The Kinase Library | Johnson et al. 2023⁶ | https://kinase- |
| | | library.phosphosite.org/ea |

Cell culture media and buffers: The names and recipes for all the buffers listed in subsequent methods sections are described here. FACS buffer: PBS supplemented with 2% FBS and 0.02% sodium azide. T cell media: RPMI 1640, 1000 FBS, penicillin/streptomycin (Gibco), 2 mM L-glutamine, 50 μM 2-mercaptoethanol. B16-gp33 media: DMEM, 10% FBS, penicillin/streptomycin (Gibco; final concentrations of 100 units/mL penicillin and 100 μg/mL streptomycin), and 100 μg/mL geneticin to maintain expression of gp33. Tumor digestion media: DMEM, 10% FBS, penicillin/streptomycin, 0.5 mg/mL collagenase type IV (Millipore Sigma), 0.1 mg/mL DNAseI.

LCMV Cl 13 Assays: LCMV Cl 13 was produced according to prior studies⁷. Mice 4-8 weeks old were infected intravenously (i.v.) with 2×10⁶ pfu LCMV Cl 13. Adoptive transfers were performed as described in the adoptive transfer section, and tissues were collected from euthanized mice and processed as described in T cell isolation. Blood was collected via retro-orbital bleeding of isoflurane-anesthetized mice. All in vivo studies were performed according to guidelines approved by the Salk Institute Institutional Animal Care and Use Committee (IACUC).

Subcutaneous tumor models: Subcutaneous B16gp33 tumor models were produced by implanting B6 mice 4-8 weeks old on their flanks with 4×10⁵ B16gp33 cells. Mice were shaved at the implant site prior to injection. B16gp33 cells were expanded and collected, at low passage number, by treating with trypsin for 3 minutes. Cells were counted, rinsed, and resuspended at a concentration of 4×10⁶ cells/mL in PBS for injection. Subcutaneous tumor models of human cancer were performed by implanting NSG mice at 8-12 weeks old on their flanks with 1×10⁶ U87 cells. Mice were shaved at the implant site prior to injection. U87 cells were expanded and collected, at low passage number, by treating with trypsin for 3 minutes. Cells were counted, rinsed, and resuspended at a concentration of 1×10⁷ cells/mL in PBS for injection. Tumor measurements were performed with calipers starting at d7 post-implantation and were performed at least twice per week until endpoint. Tumor volumes were calculated from caliper measurements assuming a prolate ellipsoid shape (V=0.5233×length×width²), where the length is the longer axis of the caliper measurements. Immediately prior to sacrifice, one final caliper measurement of tumor size was taken. All in vivo studies were performed according to guidelines approved by the Salk Institute IACUC.

T cell isolation from spleen or tumor: T cells from spleen: Spleens were collected from euthanized animals and immediately placed in T cell media on ice. Spleens were gently mashed on a 70 μm nylon filters using a 1 mL syringe, and the filters were washed generously with T cell media. Splenocytes were spun down at 500 rcf, media was removed, and red blood cells were lysed by incubating the splenocytes in 1 mL ACK buffer for 4 min at room temperature. Lysis was quenched by addition of 5 mL T cell media. Cells were resuspended in T cell media and prepared for experimental use. T cells from tumor: Subcutaneous tumors were dissected from mice, weighed, diced with razor blades in a 60 mm petri dish, and placed on ice. The tumor slurry was suspended in 5-10 mL of tumor media and shaken at 200 rpm in a shaker incubator at 37° C. for 30 min. The slurry was then mashed through a 70 μm nylon filter using a 1 mL syringe plunger to produce a single cell suspension. The filter was then rinsed with extra tumor media to collect all the cells. The cell suspension was spun at 450 rcf for 3 min at 4° C., the supernatant was poured off, and the pellet was treated with 2 mL ACK buffer for 5 min at room temperature to lyse red blood cells. The ACK lysis was quenched with 10 mL tumor media, and cells were counted and then used for further experiments. All in vivo studies were performed according to guidelines approved by the Salk Institute IACUC.

Flow cytometry and antibodies: Following isolation of cells, single cell suspensions were stained with anti-CD16/32 (BioLegend) on ice for 15 min to block Fc receptors. Antibody cocktails for surface proteins were prepared up to 1 h in advance. Antibodies were added to FACS buffer, mixed well, and kept on ice. Immediately before staining cells, LIVE/DEAD Fixable viability stain was added to the antibody cocktail, mixed well, and cells were stained. For stains of 3×10⁶ cells or fewer, cells were stained in 50 μL of Ab cocktail; for stains greater than 3×10⁶ cells, the staining volume was increased to 100 μL. Surface and viability stains were done for 30 min on ice. Cells were rinsed once in FACS buffer. Centrifugation to collect cells and exchange incubation media was done at 450 rcf for 3 min at 4° C. for unfixed cells; after fixation, described below, centrifugation was done at 550 rcf, again for 3 min at 4° C. To measure cytokine production, cells were resuspended in 200 μL T cell media and stimulated. For LCMV experiments, P14 T cells were stimulated with 100 ng/mL LCMV gp33 (KAVYNFATC) in the presence of 5 μg/mL Brefeldin A (BioLegend 420601) for 5 h at 37° C.; for tumor experiments, T cells were instead stimulated with 10 ng/mL PMA and 1 μg/mL ionomycin for 4 h. For intracellular staining of cytokines, cells were fixed for 1 min at room temperature with BioLegend Fixation Buffer (PBS with 4% paraformaldehyde, catalog number 420801). Cells were spun down, the fixation buffer was removed, and then cells were incubated for 30 min on ice in FoxP3 Transcription Factor Fixation/Permeabilization buffer. For intracellular stains of panels that did not include cytokines, the 1 min fixation step was omitted. Following fixation/permeabilization, cells were rinsed once in eBioscience Permeabilization buffer, spun down, and then stained with antibodies against other intracellular proteins. The intracellular Ab cocktail was made in Permeabilization buffer and cells were stained for 45 min on ice. Cells were rinsed twice with Permeabilization buffer, and then if required (such as for PKC theta or eta), stained with a fluorescently conjugated secondary antibody in Permeabilization buffer for 30 min on ice. After secondary Ab staining, cells were rinsed two more times in Permeabilization buffer. Then when staining was complete, cells were resuspended in FACS buffer and prepared for flow cytometric analysis. Samples were processed on an LSR II or A3 flow cytometer (BD Biosciences) and data were analyzed with FlowJo v10. The antibodies used for staining are listed in Table A.

In vitro T cell exhaustion assays: In vitro T cell exhaustion assays were performed by continuously co-culturing P14 T cells with B16-gp33 cells. This protocol is conceptually similar to previously published protocols^{8,9}. The timeline of the assay is referred to relative to days post-activation for the T cells. Day −2: B16-gp33 cells were started fresh from liquid nitrogen storage or split at low passage number into a new flask containing B16 media. Day 0: P14 T cells were activated with 100 ng/mL gp33 and 10 ng/mL recombinant murine IL-2. B16-gp33 cells were split to have one T175 flask. Day 1: if genetic modification of T cells was required, it was performed following the protocols described below in the CRISPR/Cas9 RNP electroporation or retroviral transduction sections. Regardless of whether genetic modification was performed or not, T cells were exchanged into fresh T cell media. Day 2: B16-gp33 cells were removed from the flask by treating them with Versene for 3 min at 37 C. The cells were exchanged into B16-gp33 media and then irradiated to induce cell cycle arrest to facilitate the maintenance of the assay over the course of a week. A dose of 45 Gray was administered by placing cells in a conical tube 25 cm from a Co-60 source for the required time (5-8 min, as the source decayed over the course of this study). Cells remained over 95% viable after irradiation. Cells were plated at 5×10⁴ cells per well of a 96 well flatbottom plate, 1.5×10⁶ cells per well of a 6 well plate, or otherwise scaled appropriately by vessel surface area. Day 3: The splenocyte mixture containing activated P14 cells was collected, and cells were counted. 2.5×10⁴ splenocytes were plated in each well, a ratio of 1:2 of splenocytes to B16-gp33 cells, roughly corresponding to 1:5 P14 to B16-gp33 cells. The B16-gp33 media, containing geneticin, was removed, and wells were rinsed with T cell media prior to addition of P14 cells in T cell media. The T cell/B16-gp33 co-culture was supplemented with cytokines or drugs as described in the main text, and the total culture volume was 200 μL in each well of a 96 well plate. The standard conditions for enforcing the most severe level of exhaustion entailed using IL-2 at 10 ng/mL and PMA at 1 g/mL (diluted 10,000× from a 10 mg/mL stock in ethanol). Days 4-6: Nothing was done to the culture on day 4. On day 5, T cells were collected from the plate using a multichannel pipette by thoroughly resuspending media in each well of the plate, plated in a round bottom plate to centrifuge the cells, and T cells were added with fresh media, cytokines, and drugs as appropriate to new wells of B16-gp33 cells. Again, B16-gp33 containing wells were rinsed with T cell media prior to addition of T cells. On day 6, this media exchange procedure was repeated. Day 7: T cells were collected with pipetting. Versene was used where necessary if T cells remained bound to surviving B16-gp33 cells in certain conditions: using Versene rather than trypsin avoids potential cleavage of surface antigens that may be stained in flow cytometry. T cells were then used for downstream assays including flow cytometry and gene expression analysis.

Chemical biology and drug treatments: Inhibitor screening of kinases downstream of PKC theta or eta: GSK-626616 (MedChem Express) was used to inhibit DYRK¹⁰ and was diluted from a 10 mM stock to a final concentration of 1 μM. AZD1208 (Selleck Chemicals) was used to inhibit PIM kinases¹¹ was diluted from a 10 mM stock in DMSO to a final concentration of 1 μM. And inhibitor. Compound 20 (Selleck Chemicals) was used to selectively inhibit PKC theta¹² and was diluted from a 50 mM stock in DMSO to a final concentration of 5 μM. Ralimetinib dimesylate (Selleck Chemicals) was used to inhibit p38¹³ and was diluted from a 10 mM stock in DMSO to a final concentration of 5 μM. WNK-IN-11 was used as a WNK inhibitor¹⁴ and was diluted from a 10 mM stock in DMSO to a final concentration of 1 μM. D4476 was used as a pan-CK1 inhibitor¹⁵ and was diluted from a 10 mM stock in DMSO to a final concentration of 10 μM. PKC theta degradation assays: To induce PKC theta degradation and/or to measure the effects on T cells of chronic PKC agonism, cells were continuously cultured in phorbol 12-myristate 13-acetate (PMA). For Nanostring gene expression analysis, T cells were incubated with PMA at a concentration of 1 μg/mL in T cell media (with 10 ng/mL IL-2) from days 3 to 7 post-activation, changing media every day. For degradation timecourse assays and PKC theta mutant screening, the same culture and PMA conditions were used, but for up to a maximum of 24 hours of PMA treatment.

Nanostring gene expression analysis was performed on cells that were subjected to the in vitro exhaustion protocol described in the above section, with minor changes. The cells for Nanostring analysis did not receive antigen beyond initial peptide-activation, but they did receive media changes, including PMA or a DMSO vehicle control, on the same schedule as the in vitro exhaustion protocol (new media, IL-2, and drugs on days 3, 5, 6, and 7, with sample collection on day 8 for this analysis). Two technical replicate wells of a flatbottom 96 well plate were pooled, and 3×10⁴-5×10⁴ cells were collected for RNA isolation (RNA Clean & Concentrator-5, Zymo #R1013). The isolated RNA was hybridized with a nCounter custom probe set (108 probes) according to the manufacturer's protocol. The hybrid RNA samples were loaded onto a Nanostring nCounter MAX Profiler. The expression data was normalized to expression of a panel of positive control housekeeping genes (Cd8a, Cd2, B2m) and internal negative control probes. Phenotypes of the cells were validated in parallel by performing flow cytometry on cells that were collected from the same samples but not used for Nanostring.

Plasmid cloning and molecular biology: Retroviral overexpression plasmids were created using 2× NEBuilder HiFi DNA Assembly master mix (New England Biolabs #2621) to perform Gibson assembly. Genes of interest were inserted into several different backbones (MigR1², Addgene #27490; MSCV-IRES-Thy1.1 DEST³, Addgene #17442). MigR1 was digested with XhoI and MSCV-IRES-Thy1.1 was digested with NotI to linearize the vectors before Gibson assembly. A Kozak sequence (GCCACC) was added before the start codon of inserted genes to facilitate translation. Long (>300 bp) DNA inserts were synthesized at Twist Bioscience and oligonucleotides were synthesized at Integrated DNA Technologies. For non-degradable Prkcq mutant screening, the WT Prkcq gene sequence was divided into 4 fragments, and WT and mutant versions of each fragment (containing Lys to Arg mutations in the region) were synthesized to facilitate mutant scanning in blocks of 4-6 Lys residues (e.g. Frag 1 (WT), Frag 2 (containing mutants K409R, K412R, K413R, K429R, K451R) Frag 3 (WT), Frag 4 (WT)). This mixing and matching of modules allows for rapid generation of many gene variants using a handful of short, directly synthesized DNA segments.

CRISPR/Cas9 deletion of genes in primary T cells: Following isolation of splenocytes, CD8 T cells were purified by negative selection. A negative selection cocktail was prepared by incubating streptavidin-coated magnetic beads with biotin conjugated Abs against CD4, MHC-II, CD11c, CD11b, Ter 19, B220, CD49b, and TCR γ/δ. For CRISPR/Cas9 RNP deletion of genes encoding the PKCs (Prkcq or Prkch, as well as the scramble sgRNA negative control used in the PKC experiments), the RNP electroporation was performed 24 h post-activation of T cells so that deletion of Prkcq would not interfere in the priming of naïve T cells. Otherwise, RNP electroporation was performed on naïve cells. Unless otherwise stated, all guide sequences were obtained from the Brie library⁸⁴, and their deletion of the target gene was validated by flow and/or Western blot. RNP complexes were formed by incubating 36 pmol Cas9 (IDT) with 300 pmol sgRNA (Synthego) in 5 mL RNAse-free water for 10 min at room temperature. As the RNP complexes formed, 2×10⁶ purified CD8 T cells were aliquoted into a 1.7 mL tube for each electroporation reaction, and cells were rinsed in PBS. When cells were ready for electroporation, they were centrifuged, and the supernatant was completely removed. Cells were electroporated using the Lonza P3 Primary Cell 4D-Nucleofector X kit according to manufacturer instructions and as described previously²⁸. Briefly, the cell pellet was resuspended in 20 L of a mixture of P3 buffer and Supplement 1, and the cell suspension was immediately transferred to a tube containing the RNP complex mixture. This mixture was again rapidly mixed via pipetting and transferred to a cuvette for electroporation using the Lonza program DN100. Following electroporation cells were removed from the cuvette and allowed to recover in 5 mL of T cell media pre-warmed to 37° C. After 15 min of recovery at 37° C., cells were pelleted and prepared for experimental use. For RNP mediated deletion of genes in primary human CD8 CAR-T cells, CD8 T cells were initially isolated from PBMCs using the EasySep CD8 negative selection kit (STEMCELL) and re-purified by FACS following lentiviral transduction. RNP complexes were formed in the same manner described above. 2×10⁶ human CAR-T cells were electroporated with their RNP complexes using the Lonza program EO-115 for ‘high functionality’ T cells. Recovery of cells following electroporation was the same as for primary mouse cells.

Retroviral transduction of primary T cells: T cells were engineered to overexpress target genes by retroviral transduction. Ecotropic retrovirus was packaged in HEK 293T cells by transfecting the HEK cells with a target plasmid of interest (described in plasmid cloning above) and pCL-Eco (Addgene #12371). The conditions listed here describe one transfection reaction worth of material, and this recipe was scaled accordingly to the amount of retrovirus needed to transduce sufficient T cells. The day before transfection, 4×10⁵ HEK cells were plated in 2 mL HEK media (DMEM, 10% FBS, penicillin/streptomycin) in one well of a 6 well plate; each well of a 6 well plate provided enough virus to transduce 3×10⁶ splenocytes from a P14 mouse or 7.5×10⁵ purified CD8 T cells. On transfection day, each transfection reaction was made in a 1.7 mL tube and contained 1 g of target plasmid, 0.5 μg pCL-Eco, and 6 μL X-tremeGENE 9 (MilliporeSigma 6365779001), and a volume of OptiMEM (ThermoFisher 31985062) to reach 100 μL (X-tremeGENE added last). The reaction was mixed by gently flicking the tube. After 15 min incubation at room temperature, the mixture was added dropwise to the HEK cells and the plate was gently swirled and then incubated at 37° C. overnight. About 18 h post-transfection, the HEK media was removed and replaced with 2 mL fresh HEK media, gently adding media along the side of the well to avoid disrupting the HEK cells. The same day of the HEK media replacement, splenocytes were isolated as above and T cells were activated. For experiments using P14 T cells, the leukocytes remaining after red blood cell lysis were resuspended in 10 mL T cell media, gp33 peptide was added to a final concentration of 100 ng/mL, and recombinant murine IL-2 (PeproTech 212-12) was added to a final concentration of 10 ng/mL. For experiments not using P14 T cells, CD8 T cells were isolated via negative selection as described above, resuspended to 3×10⁶ cells/mL in T cell media (with 10 ng/mL IL-2), and activated via platebound anti-CD3/28 stimulation. Once T cells were placed in their activation media, they were incubated at 37° C. overnight. The following day, 18-24 hours post-activation, the retrovirus containing supernatant was collected from HEK 293T cells and centrifuged at 300 rcf at room temperature for 10 minutes to remove any stray HEK cells. For each transduction, T cells were counted and adjusted to plate 3×10⁶ activated P14 splenocytes or 7.5×10⁵ purified CD8 T cells per well in a 12 well plate, and the retrovirus from one transfection was added to each well of T cells. Polybrene (MilliporeSigma TR-1003) was added to a final concentration of 9 g/mL and gently mixed. T cells were transduced by centrifuging the transduction mixtures at 1500 rcf for 90 minutes at 32° C.; the brake speed of the centrifuge was reduced to avoid disturbing nascent retroviral infections. Following the spin, the transduction plate was gently removed from the centrifuge and incubated at 37° C. for 3 h to allow infection to complete. After this 3 h rest, transductions were spun again at 600 rcf at 32° C. for 3 min to adhere T cells to the plate, and the media was gently removed. The transduced T cells were then either given fresh T cell media with 10 ng/mL IL-2 if they were to be used for in vitro experiments, or they were prepared for adoptive transfer for in vivo experiments. The transduction protocol used here is based on prior work¹⁸.

Lentiviral transduction of primary human T cells: Primary human CD8 T cells were engineered to express a chimeric antigen receptor (CAR) that recognizes GD2. The conditions listed here describe one transduction reaction worth of material, and this recipe was scaled accordingly to the number of T cells needed for downstream experiments. A vial of 1×10⁷ frozen PBMCs from a healthy human donor was thawed and plated in 5 mL of T cell media on a plate that had been pre-coated with anti-CD3 (1 μg per well) and anti-CD28 (2 μg per well). T cells underwent platebound activation for 48 hours. 24 hours after plating PBMCs, a low-binding 24 well plate was prepared for transduction by pre-coating wells with retronetcin (Takara). For each well of PBMCs being activated, a well of a 24 well plate was coated with 25 μL of 1 mg/mL retronectin diluted into 1 mL of PBS. The retronectin plate was incubated overnight at 4° C. The following day, shortly before transduction, the retronectin solution was removed, and the plate was additionally coated for half an hour at room temperature with a sterile solution of 1 mL of 2% BSA in PBS per well. 48 hours post-activation, PBMCs containing activated T cells were collected by gentle pipetting. CD8 T cells were isolated by magnetic bead-based negative selection using the EasySep CD8 negative selection kit (STEMCELL) and counted. For each retronectin-coated well, 1×10⁶ purified CD8 T cells were added to the well in 400 μL of T cell media. Then, 1 μL of lentivirus stock (concentrated 100× from virus-containing supernatant using the Retro-X concentrator) was added to each well. The plate containing virus, retronectin, and cells was spun at 1500 g for 90 minutes at 32° C., with a gentle brake setting to avoid disturbing nascent infections. The plate was gently removed from the centrifuge and incubated at 37° C. for 3 hours, after which 1 mL of T cell media was gently added to each well. Human IL-2 was added to a final concentration of 10 ng/mL. The plate of cells was incubated overnight at 37 Celsius. The following day (72 hours post-activation), T cells were collected by centrifugation and plated in 2 mL of fresh T cell media with 10 ng/mL human IL-2. T cells were allowed to expand for 2-3 more days (until day 5-6 post-activation), at which point FACS was performed to sort out CAR transductants based on a fluorescent reporter expressed from the lentiviral vector. Cells were expanded for 2-3 more days once again prior to RNP electroporation as described. Ethical approval for this study was granted by the Institutional Review Board [protocol: #22-0006]. Informed consent was obtained from the participant prior to sample collection.

Adoptive T cell transfer: To prepare T cells for adoptive transfer, the cells were rinsed once in PBS and then resuspended in PBS. T cells were always transferred via retro-orbital injection of 100 μL per mouse. The activation state of the T cells, the timing of transfer, and the number of cells were adjusted accordingly for each immune challenge as follows. For all LCMV Cl 13 experiments, 1.5×10⁴ purified T cells or 7.5×10⁴ transduced P14 splenocytes were adoptively transferred. T cells that were either transduced or were electroporated 24 h post-activation were transferred into mice that had also been infected 24 h prior, so as to match the activation timeline for the transferred and endogenous T cells. For T cells that were electroporated while naïve, they were transferred into mice 24 h before the mice were infected. For subcutaneous B16gp33 tumor experiments, T cells were transferred on day 10 post-implantation, when tumors had become palpable. Unless otherwise stated, 2×10⁵ purified T cells (or 1×10⁶ P14 splenocytes) were transferred 24 h post-activation. For subcutaneous U87 tumor experiments, T cells were transferred at approximately day 10 post-implantation, when tumors had reached a mean volume of 50-70 mm³ based on caliper measurements. Each mouse received 9×10⁵ GD2 CAR-T cells. The CAR-T cells had been FACS-sorted to yield a pure CAR+ population, which were additionally treated with CRISPR RNP to delete target genes prior to adoptive transfer.

Phosphoproteomics: CD8 T cells were negatively selected and pooled from 5 animals, activated overnight by platebound anti-CD3/28 stimulation, and subsequently electroporated with RNP complexes containing guides against Prkcq, Prkch, or a scrambled non-targeting control. Cells were expanded in vitro until 6 days post-activation and then they were prepared for re-stimulation for phosphoproteomic analysis. Prior to sample collection, 5×10⁷ cells per genetic background were aliquoted per re-stimulation condition. Cells were plated on 10 cm dishes with or without anti-CD3 coating. Cells were re-stimulated for 30 min and then immediately placed on ice and cells were collected. The cell pellets were spun down, the supernatant was removed, the pellets were snap frozen in liquid nitrogen. Phosphoproteomics data were generated at the Biomolecular and Proteomics Mass Spectrometry Facility at UCSD. Sample preparation for mass spectrometry: Samples were lyophilized overnight and reconstituted in 200 μl of 6M Guanidine-HCl per 1 mg of protein. The samples were then boiled for 10 minutes followed by 5 minutes cooling at room temperature. The boiling and cooling cycle was repeated a total of 3 cycles. The proteins were precipitated with addition of methanol to a final volume of 90% followed by vortexing and centrifugation at maximum speed on a benchtop microfuge (14,000 rpm) for 10 minutes. The soluble fraction was removed by flipping the tube onto an absorbent surface and tapping to remove any liquid. The pellet was resuspended in 200 μl of 8 M urea made in 100 mM Tris-Cl pH 8.0. TCEP was added to a final concentration of 10 mM and chloro-acetamide solution was added to final concentration of 40 mM and vortexed for 5 minutes. The solution was then acidified using TFA (0.5% TFA final concentration). 3 volumes of 50 mM Tris pH 8.0 were added to the sample to reduce the final urea concentration to 2 M. Trypsin was added in a 1:50 ratio and incubated at 37° C. for 12 hours. The solution was once again acidified using TFA (0.5% TFA final concentration) and mixed. Samples were desalted using Thermo C18-Stage Tips (cat #87782 and #87784) and 100 mg C18-solid phase extraction (Waters cat #WAT023590) as described by the manufacturer protocol. The eluted peptides are dried in the speed vac. The peptide concentration of sample was measured using BCA. To prepare proteins for global phosphoproteomics, phospho-peptides were enriched using High-Select Fe-NTA Phosphopeptide Enrichment (A32992 Thermo Scientific). The enriched phosphopeptide fraction was then further fractionated using Pierce™ High pH Reversed-Phase Peptide Fractionation Kit (Pierce™ High pH Reversed-Phase Peptide Fractionation Kit Catalog number: 84868). Fractionation protocol was performed as described by the manufacturer kit. Eight fractions were generated from this step. LC-MSMS analysis: Trypsin-digested peptides were analyzed by ultra high pressure liquid chromatography (UPLC) coupled with tandem mass spectroscopy (LC-MS/MS) using nanospray ionization. The nanospray ionization experiments were performed using a TimsTOF 2 pro hybrid mass spectrometer (Bruker) interfaced with nano-scale reversed-phase UPLC (EVOSEP ONE). Evosep method of 30 SPD (samples per day) was utilized using a 10 cm×150 m reverse-phase column packed with 1.5 μm C18-beads (PepSep, Bruker) at 58° C. The analytical columns were connected with a fused silica ID emitter (10 μm ID; Bruker Daltonics) inside a nanoelectrospray ion source (Captive spray source; Bruker). The mobile phases comprised 0.1% formic acid in water as solution A and 0.1% FA/99.9% acetonitrile as solution B. The mass spectrometry setting for the TimsTOF Pro 2 are as following: PASEF method for standard proteomics. The values for mobility-dependent collision energy ramping were set to 95 eV at an inversed reduced mobility (1/ko) of 1.6 V s/cm² and 23 eV at 0.73 V s/cm². Collision energies were linearly interpolated between these two 1/ko values and kept constant above or below. No merging of TIMS scans was performed. Target intensity per individual PASEF precursor was set to 20 000. The scan range was set between 0.6 and 1.6 V s/cm² with a ramp time of 166 ms. 14 PASEF MS/MS scans were triggered per cycle (2.57 s) with a maximum of seven precursors per mobilogram. Precursor ions in an m/z range between 100 and 1700 with charge states ≥3+ and ≤8+ were selected for fragmentation. Active exclusion was enabled for 0.4 min (mass width 0.015 Th, 1/ko width 0.015 V s/cm²). Protein identification and label free quantification⁵ was carried out using Peaks Studio X (Bioinformatics solutions Inc.). Peptide peak area files were analyzed in R. Kinase motifs were determined using the Kinase Library⁶. All proteomics in this manuscript were performed at the UC San Diego Biomolecular and Proteomics Mass Spectrometry Facility (BPMSF).

Western blotting: Cell pellets for Western blots were collected by spinning cells at 600 rcf in a centrifuge cooled to 4° C., and then the pellets were stored at −80° C. prior to lysis. Lysis buffer was prepared by supplementing RIPA buffer with complete protease inhibitor cocktail and PhosSTOP phosphatase inhibitor at the manufacturer's recommended concentrations. For each 1 million cells in the cell pellets, 25 μL lysis buffer was added to cell pellets, gently mixed, and left on ice for 10 minutes. The lysate was then centrifuged at 18,000 rcf at 4° C. for 10 minutes. The supernatant was removed and mixed with an appropriate volume of 4× Laemmli buffer and 2-mercaptoethanol (final concentration 2.5% v/v). The solution was mixed and then boiled for 10 minutes. After cooling, samples were loaded onto 4-15% gradient acrylamide gels and subjected to electrophoresis at 100V for approximately 1 hour. Proteins in the gel were transferred to a PVDF membrane in Towbin buffer at 100V for 90 minutes. Following transfer, membranes were blocked in a solution of 5% w/v BSA in TBST buffer at room temperature for 1 hour. The membranes were blotted with primary antibodies overnight at 4° C., rinsed 3 times in TBST at room temperature for 10 minutes and then blotted with an appropriate horseradish peroxidase (HRP) conjugated secondary antibody at room temperature for 1 hour. The membranes were rinsed in TBST for 10 minutes 3 times and then were imaged via chemiluminescence. Protein content was normalized to the loading control in each lane.

Quantification and statistical analysis: Cell biology and in vivo experiments: Statistical significance of bar graphs and dot plots was assessed with Student's t-test (2 conditions) or one way ANOVA (3 or more conditions). Kinetic experiments (T cell co-transfer experiments in LCMV Cl 13 infection, tumor growth curves) were assessed by t-test or one way ANOVA at each timepoint as appropriate. Data are represented in figures as mean±s.e.m. Phosphoproteomics: Peptide sequences and their peak areas were extracted from PEAKS software. Analysis of peak area data were carried out in Rstudio as follows. Non-phospho-peptides were removed from the dataset to focus analysis on phosphorylation. Differential phosphorylation was calculated as the log 2 fold change (log 2FC) between two experimental conditions, e.g. anti-CD3 stim of sgPrkcq cells vs anti-CD3 stim of sgPrkch cells. These log 2FC values for each peptide were then normalized by the log 2FC of the unstimulated conditions for the same genotypes, e.g., unstimulated sgPrkcq vs sgPrkch, to account for proteomic drift during expansion of the distinct knockout genotypes. The normalized log 2FC values for each phospho-peptide were then used as input for the Enrichment analysis feature of the Kinase Library software⁶ with default settings.

### Example 1: Engineering a Non-Degradable PKC Theta by Scanning Mutagenesis of Lysine Residues

This example demonstrates that a non-degradable PKC theta mutant was created that enhances T cell effector function.

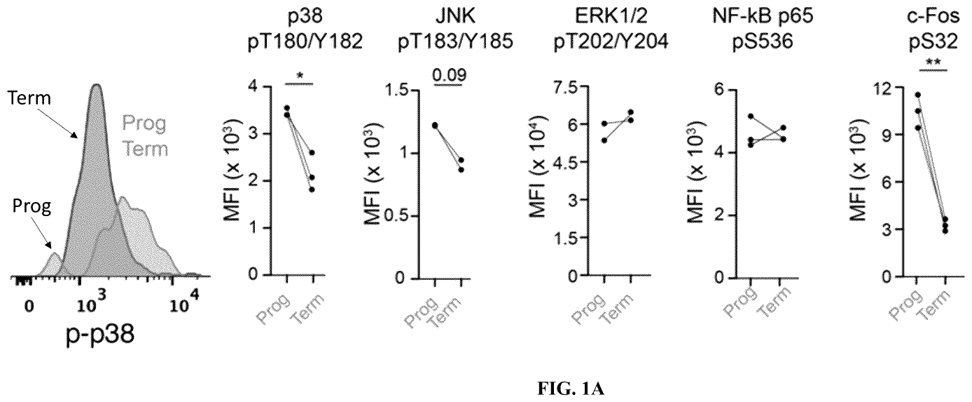

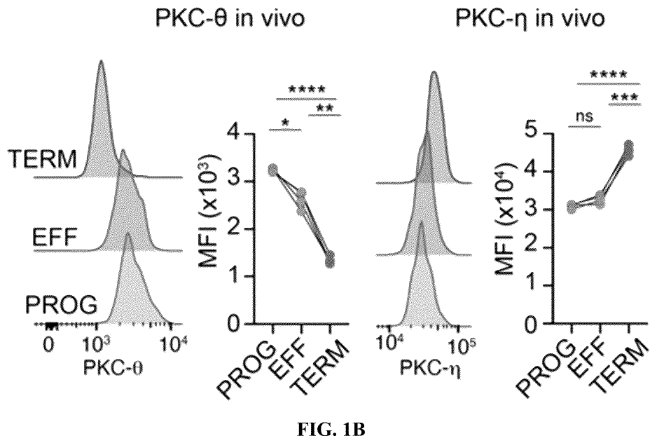

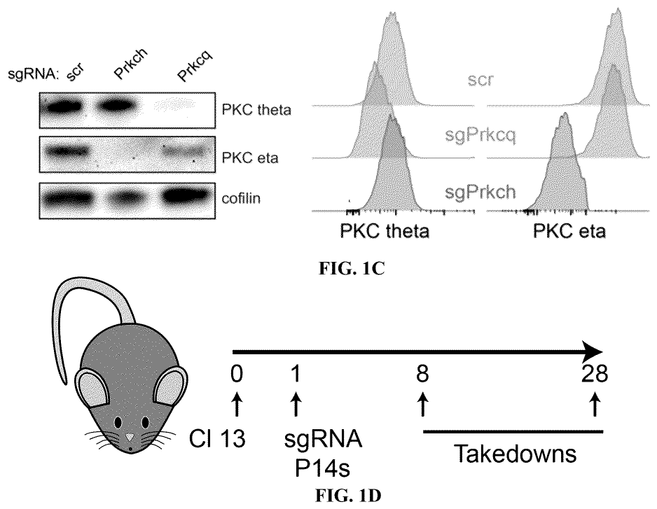

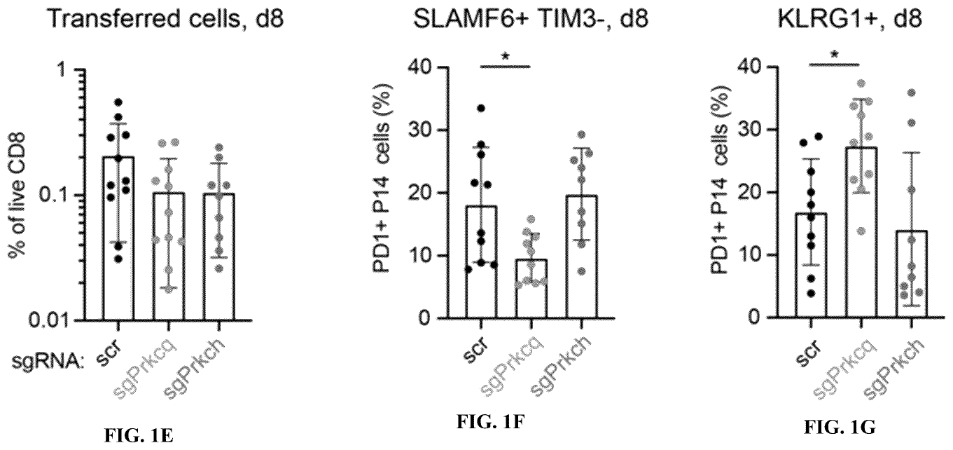

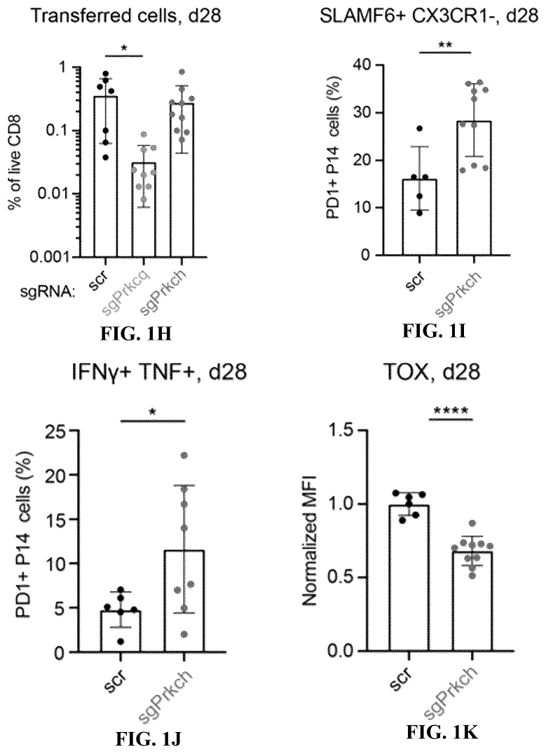

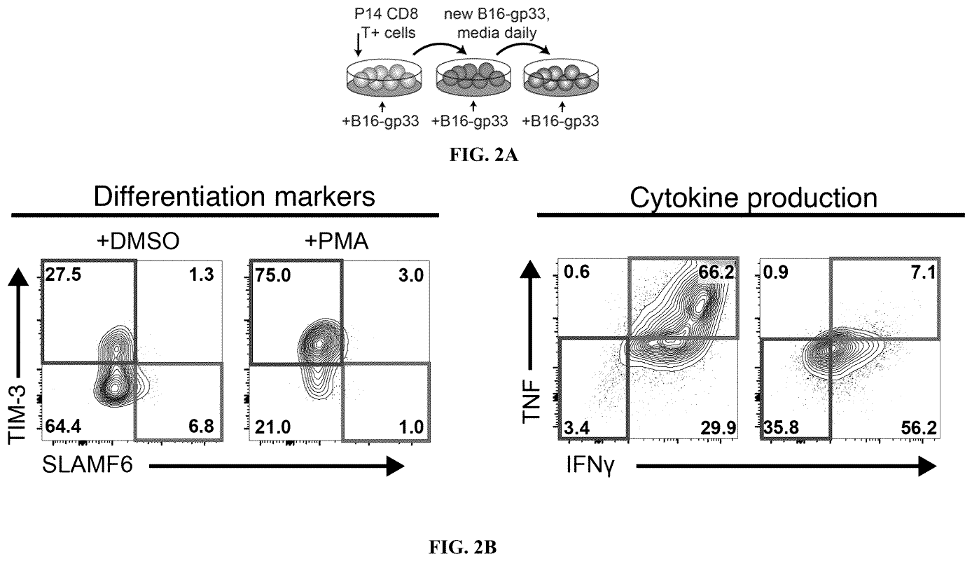

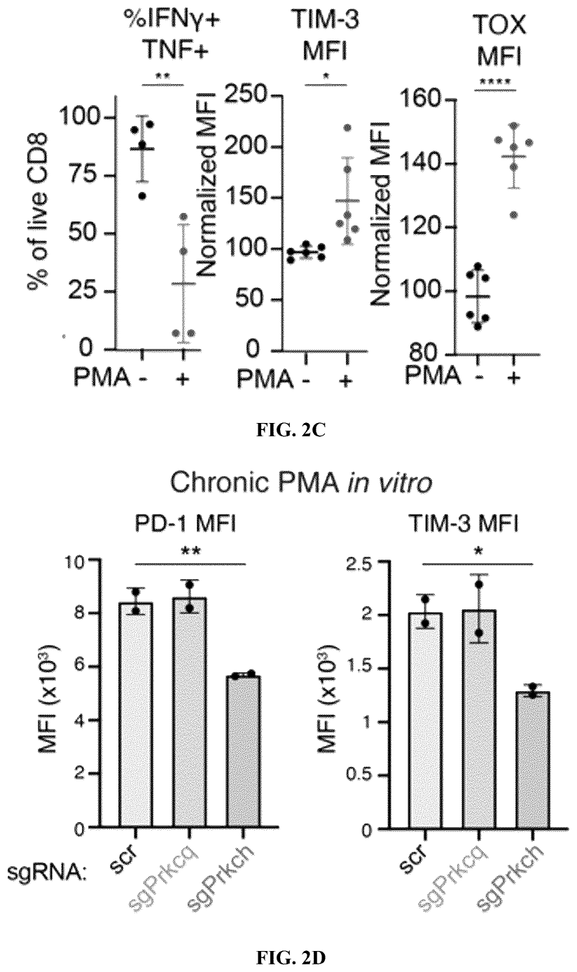

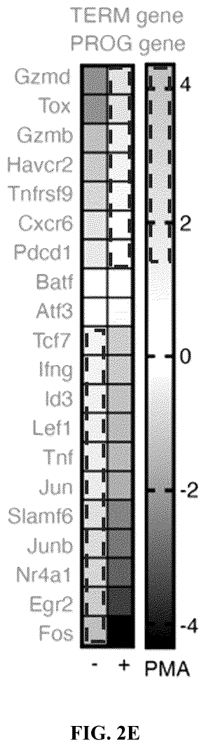

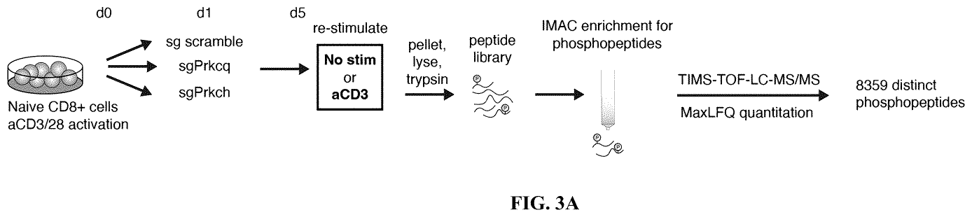

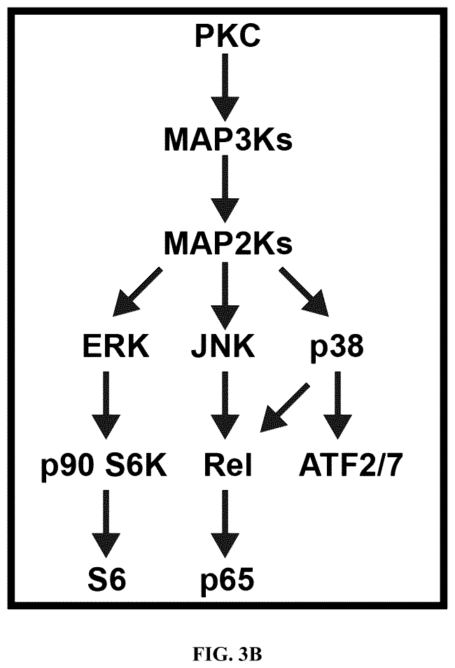

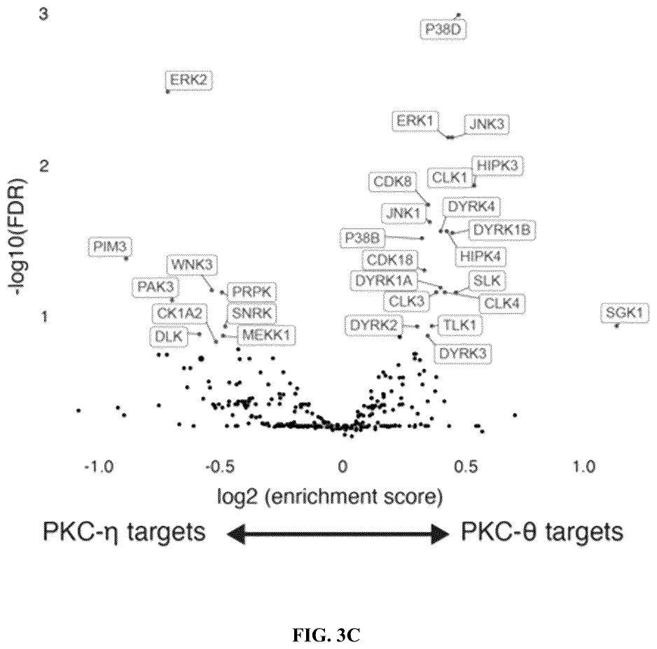

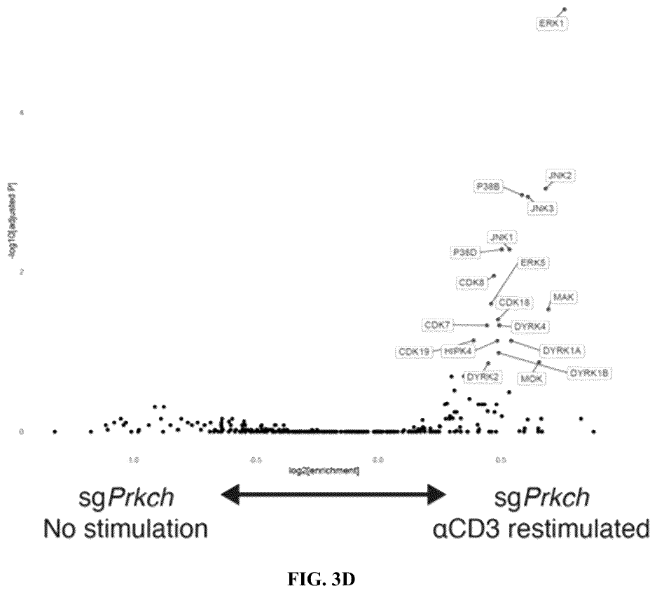

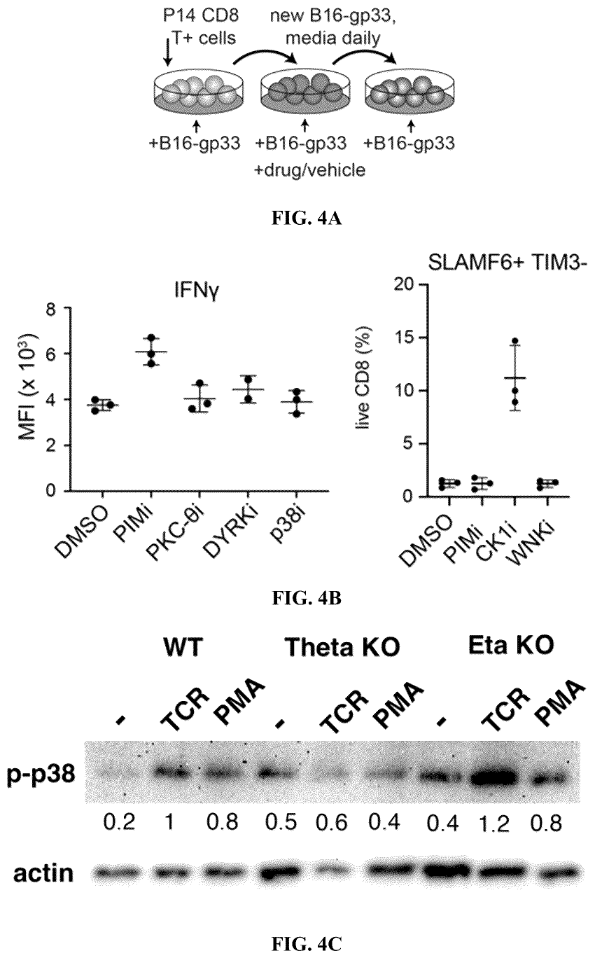

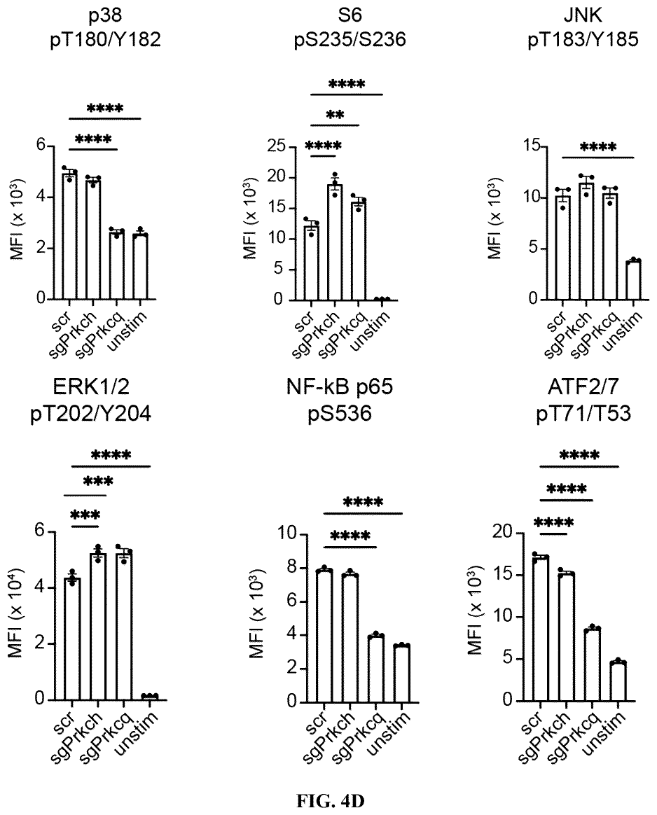

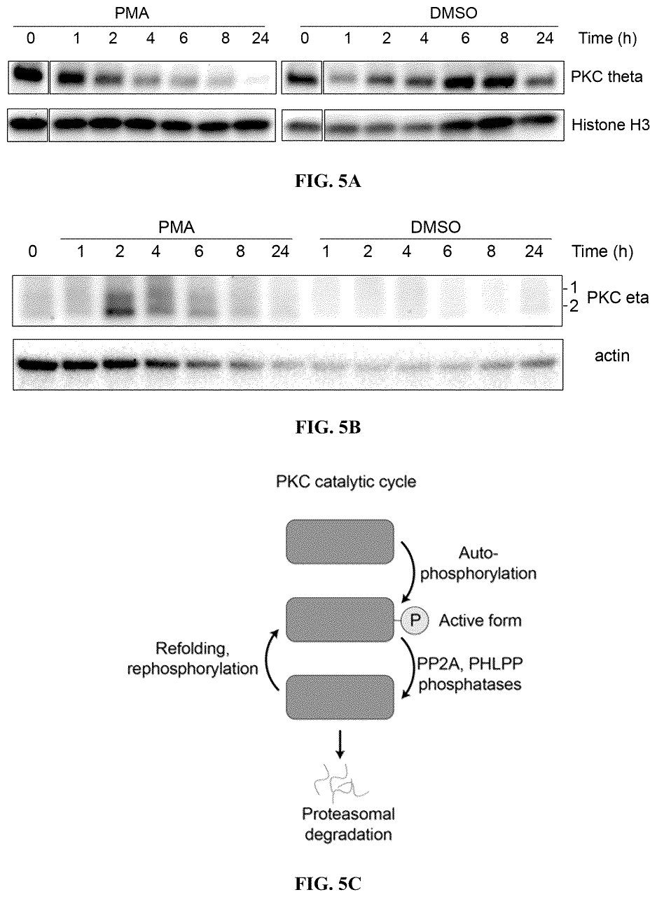

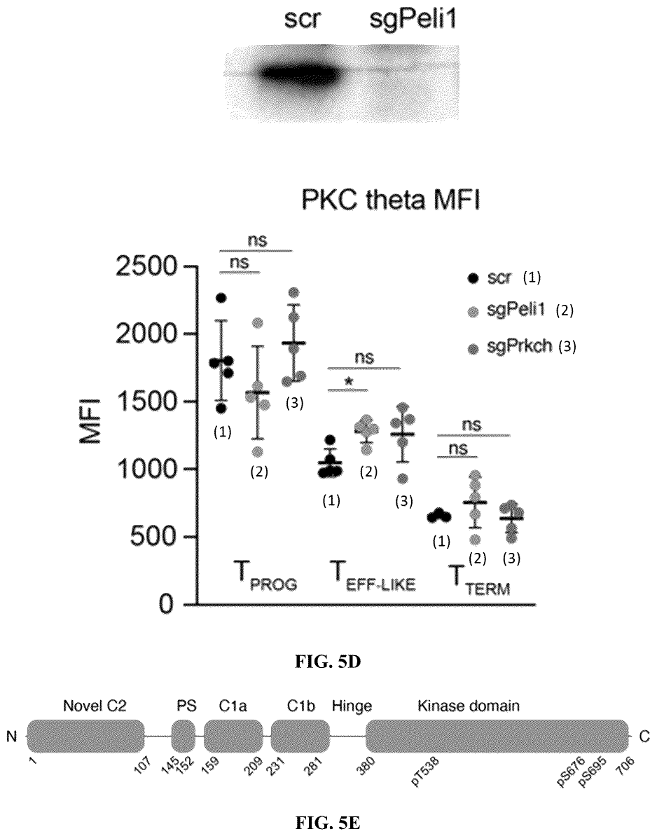

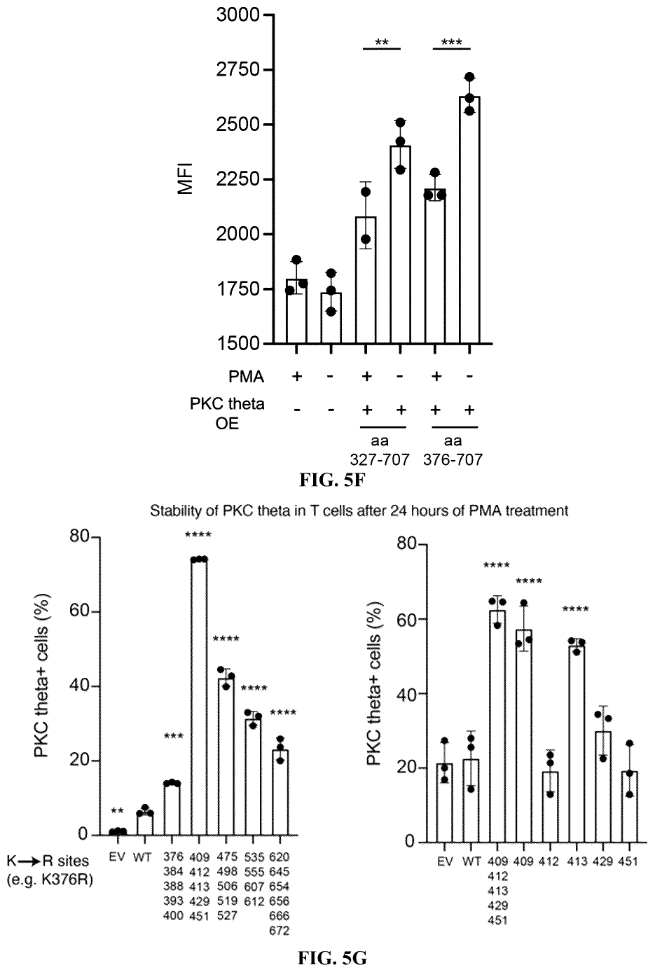

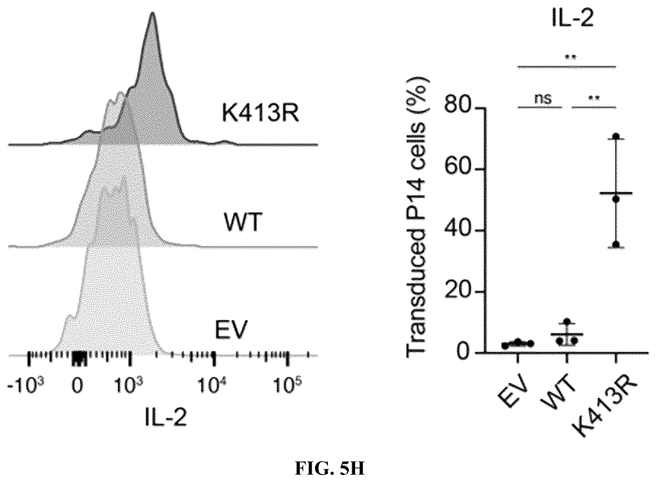

As shown in FIG. 1A-1K, PKC theta is necessary for T_{PROG} and PKC eta opposes T_{PROG} and T cell effector functions. As shown in FIGS. 2A-2E, central features of T cell exhaustion (inhibitory receptor expression, loss of cytokine production upon restimulation, expression of TOX) can be reconstituted in vitro by continuously co-culturing T cells with tumor cells that express their cognate antigen. Exhaustion phenotypes are exacerbated by continuous stimulation of these T cells with PMA, in a PKC eta-dependent manner. These findings illustrate that this in vitro co-culture system can be used to recapitulate exhaustion and can be used as a screening platform for treatments that affect T cell exhaustion. As shown in FIGS. 3A-3D, global phosphoproteomics on restimulated T cells demonstrate that PKC theta and eta regulate the activities of different downstream kinases. Compared to PKC eta, PKC theta preferentially activates canonical downstream PKC targets such as p38 and JNK family proteins, while PKC eta promotes the activity of CK1, PIM, and WNK family kinases. As shown in FIG. 4A-4D, inhibitors of kinase families downstream of PKC theta or eta were administered to T cells undergoing in vitro exhaustion, and an inhibitor (D4476) of CK1 was found to maintain T cell function during in vitro exhaustion. Additionally, Western blotting and phospho-flow cytometry were performed to validate the PKC-dependence of several substrates downstream of PKC theta. These findings suggest that the degradation of PKC theta and maintenance of PKC eta during chronic stimulation, as shown in FIGS. 5A-5B, could be a mechanism that drives T_{TERM} differentiation. As shown in FIG. 5C, PKCs autophosphorylate to achieve an activated conformation. PKC kinase activity then can be downregulated by dephosphorylation of PKCs by phosphatases such as by PP2A or PHLPP. The dephosphorylated but active conformation of PKC can then either enter a new activation cycle or be targeted for proteasomal degradation. As proteasomal degradation plays a central role in the negative regulation of PKCs, it suggests that a non-degradable variant of PKC theta would provide superior T cell function during periods of prolonged stimulation.

E3 ligases target proteins for degradation by fusing ubiquitin to lysine residues¹⁹. It was previously shown that PELI1 could function as an E3 ligase for PKC theta²⁰, and the present analysis confirmed that deletion of Peli1 increased levels of PKC theta in the TEFF-LIKE subset (FIG. 5D). More specifically, it was found that the kinase domain, at the C-terminus of PKC shown in the diagram in FIG. 5E, could be degraded during chronic PMA treatment (FIG. 5F), indicating that it contains a lysine residue sufficient to mediate degradation. To identify the lysine residue(s) responsible for degradation, PKC variants were designed by systematically mutating every lysine residue in the KD, first scanning the protein in sets of 4 to 6 residues at a time. Each site was mutated to arginine to preserve a positive charge while blocking ubiquitination. A non-degradable PKC would resist degradation during chronic stimulation. PKC variants were transduced into CD8 T cells which were stimulated with PMA for 24 hours and then the abundance of PKC theta was measured. As expected, PMA treatment was sufficient to induce degradation of overexpressed WT PKC theta (FIG. 5G). Strikingly, one variant (encoding mutations K409R, K412R, K413R, K429R, and K451R) exhibited PKC theta abundance far above the other constructs as shown in FIG. 5G left panel, though several other variants showed partial maintenance of PKC theta levels during prolonged stimulation. PKC theta constructs with individual K409R, K412R, K413R, K429R, and K451R mutations were generated and tested for PMA-induced degradation resistance (FIG. 5G right panel). Interestingly, two mutations, K409R and K413R, showed similar degradation resistance to the complete set of five mutations from which they were isolated. However, K409R has previously been identified as a catalytically dead version of PKC theta: K409 contacts ATP in the binding pocket, and this mutation interferes with ATP binding²¹. Meanwhile, K413R points out into solution in the structure of PKC theta, suggesting it as a ubiquitylation target²².

To test whether the degradation resistant PKC theta K413R improved T cell function, P14 cells were transduced with PKC theta K413R and these cells were subjected to in vitro exhaustion, including chronic PMA treatment, for 4 days. Indeed, PKC theta K413R transductants were dramatically improved in their capacity to produce IL-2 upon restimulation compared to cells transduced with EV or WT PKC theta constructs (FIG. 5H). It was also found that K413R increased the frequency of IFNγ+ TNF+ cells in the same strenuous in vitro exhaustion conditions (FIG. 5I).

These results show that PKC theta K413R is a degradation resistant variant that can maintain high levels of T cell effector function even during periods of intense, prolonged stimulation. Accordingly, the PKC theta K413R variant of the present disclosure is useful in compositions and methods for treating or preventing T cell exhaustion.

### Example 2: Overexpression of PKC Theta K413R Drives Increased Expansion of T Cells in Chronic Infection

This example demonstrates that expression of the non-degradable PKC theta K413R mutant in T cells lead to improved expansion in an in vivo chronic infection model compared to expression of WT PKC theta.

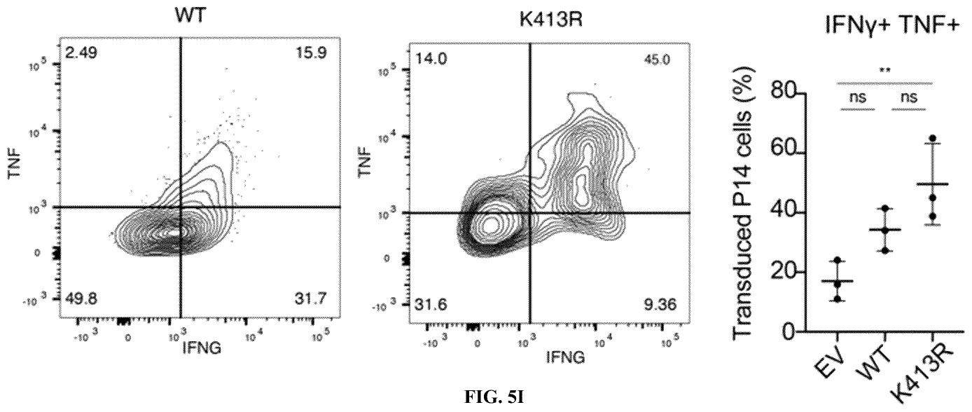

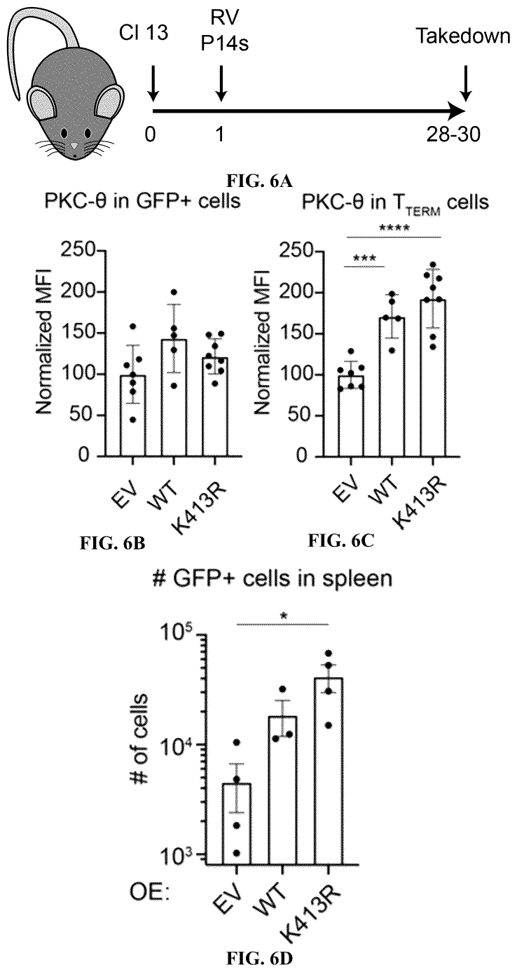

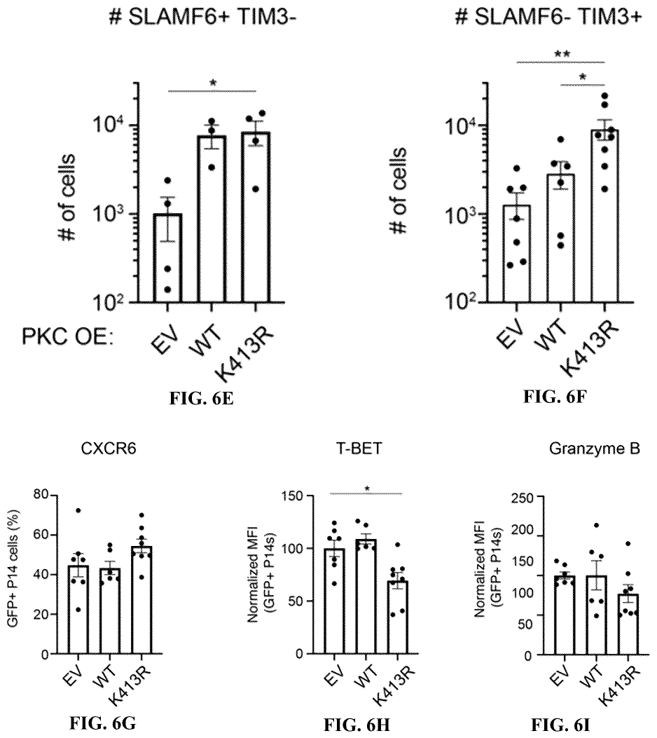

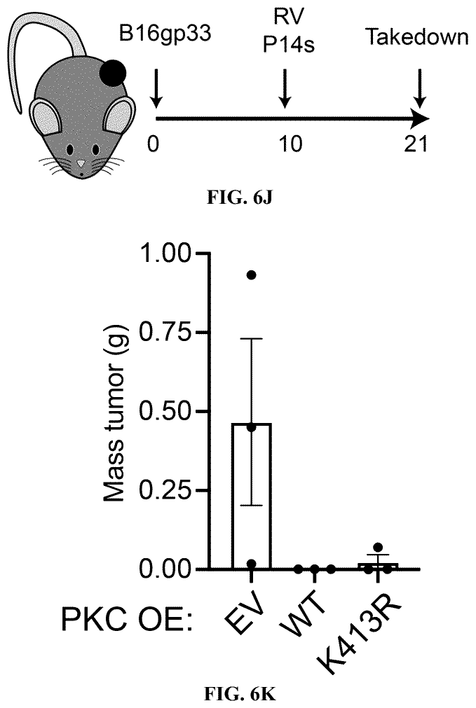

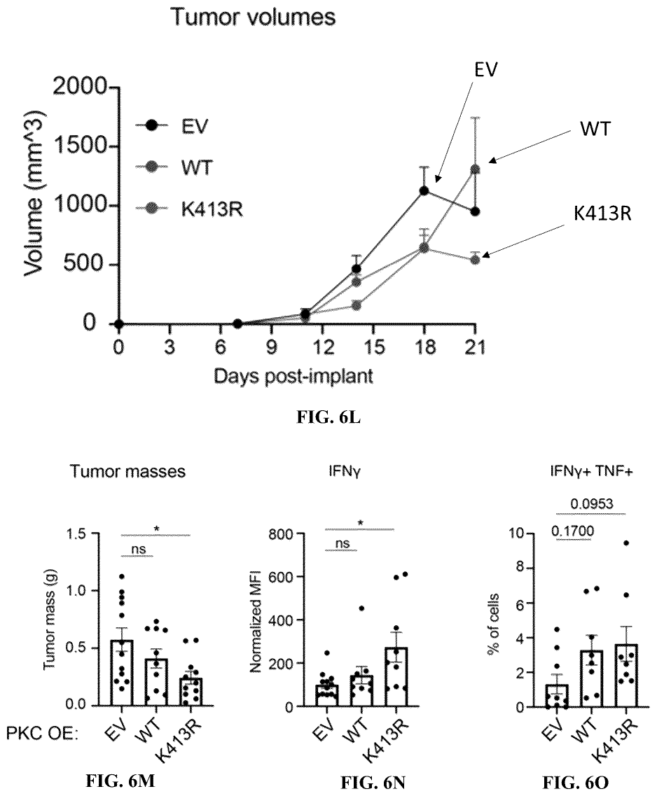

P14 cells expressing PKC theta WT, K413R, or an empty vector (EV) were adoptively transferred into control mice infected with LCMV Cl 13 (FIG. 6A). While PKC theta MFIs were not significantly changed across all GFP+ transductants, they were significantly increased in T_{TERM} cells, particularly for PKC theta K413R (FIGS. 6B-6C). Strikingly, OE of PKC theta K413R caused a roughly tenfold increase in the total number of GFP+ transductants recovered from the spleen (FIG. 6D), and a trend towards increased cell counts for OE of PKC theta WT. The two PKC theta OE vectors, WT and K413R, differed in their capacities to boost individual cell subsets. While OE of WT and K413R increased the number of T_{PROG} by similar extents relative to EV, K413R was unique in also dramatically increasing the number of T_{TERM} cells (FIGS. 6E-6F). Other measures of T cell effector function remained unchanged (FIGS. 6G-6I), indicating that the primary effect of OE PKC theta, particularly K413R, in the LCMV Cl 13 infection model is to yield a much larger Ag-specific population.

These results show that the PKC theta K413R variant of the present technology is useful for driving the expansion of T cell populations in a chronic infection model of T cell exhaustion. Accordingly, the PKC theta K413R variant of the present disclosure is useful in compositions and methods for treating or preventing T cell exhaustion.

### Example 3: Overexpression of PKC Theta K413R Provides Superior Tumor Control

This example demonstrates that PKC theta K413R expressing T cells have increased therapeutic efficacy against tumors in an in vivo model.

P14 cells expressing PKC theta WT, K413R, or an empty vector (EV) were adoptively transferred into B16gp33 tumor-bearing mice (FIG. 6J). Cells were transferred at d10 post-tumor implant, when the tumor had become palpable. When 4×10⁵ P14 cells were adoptively transferred, the cells overexpressing PKC theta WT or K413R were sufficient to completely reject tumors, while cells transduced with EV failed to control tumors (FIG. 6K).

To determine whether PKC theta K413R expression in T cells conferred an advantage relative to PKC theta WT expression, as was observed in LCMV Cl 13 infection in Example 2, the number of transferred P14 cells was reduced to 2×10⁵. At this reduced level of P14 transfer, WT PKC theta lost its advantage over EV-transduced P14 cells, while K413R P14 cells maintained a higher level of tumor control with lower tumor volume and smaller tumor masses (FIGS. 6L-6M). Improvements in T cell effector function differed somewhat between LCMV Cl 13 and tumor settings, as tumor infiltrating cells expressing PKC theta K413R had an increased level of IFNγ expression and trended towards more TNF production (FIGS. 6N-6O).

Together, these data indicate that PKC theta overexpression improves T cell function in chronic viral infection or in tumors. Critically, preventing degradation of PKC theta by introducing the K413R mutation led to even greater improvements over WT PKC theta overexpression. These findings underscore that the loss of expression and proteolytic degradation of PKC theta are key steps in the progression of T cell exhaustion and that artificially maintaining PKC theta is sufficient to boost T cell effector function. Accordingly, the PKC theta K413R variant of the present disclosure is useful in compositions and methods for treating or preventing T cell exhaustion regardless of the cause.

### Example 4: Deletion of the PKC Eta Putative Target CK1G2 Improves the TEX Response to Virus and Cancer

This example demonstrates that T cells with disrupted CK1G2 gene activity have increased therapeutic efficacy against virus and tumors in an in vivo model.

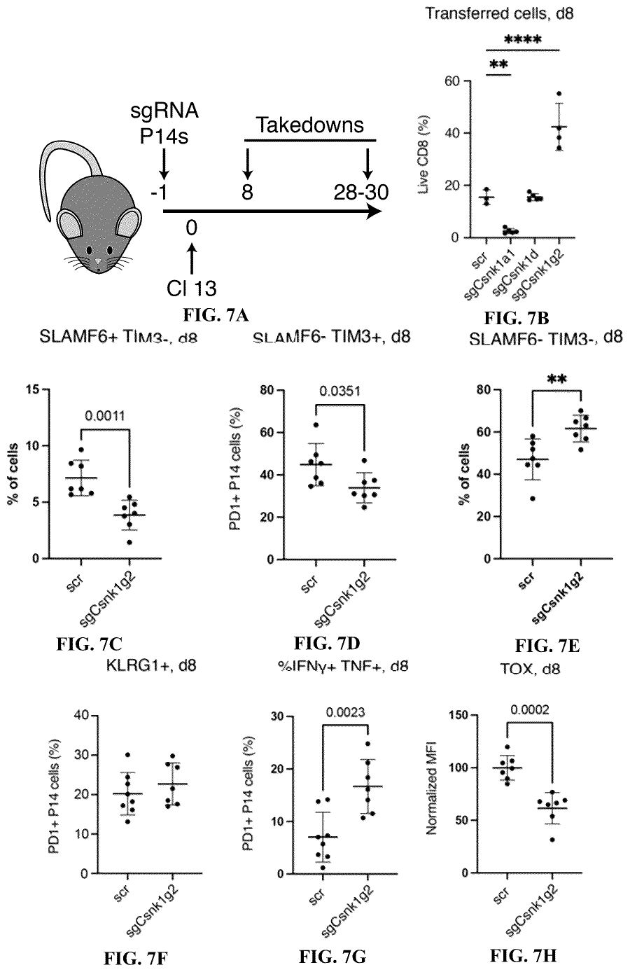

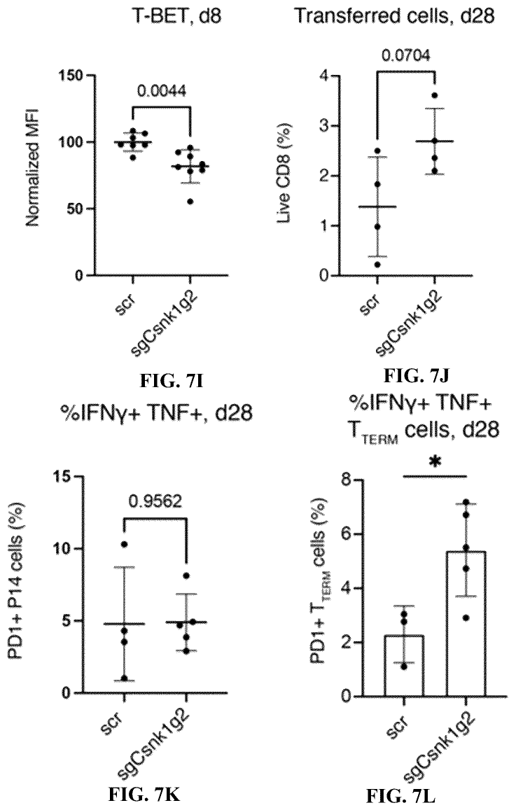

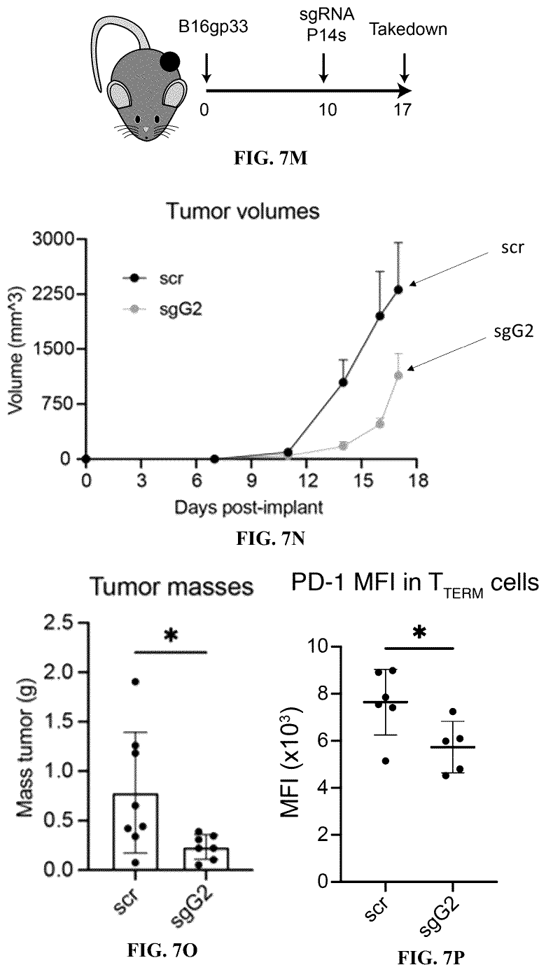

As shown in FIGS. 4A-4D, CK1 proteins are putative targets of the pro-exhaustion kinase PKC eta. Primary P14 T cell knockouts were made using CRISPR/Cas systems for each of the CK1 genes are expressed in CD8 T cells during chronic infection (Csnk1a1, Csnk1d, Csnk1g2). P14 cells bearing these deletions or a non-targeting guide (“scr”) were adoptively transferred into mice, which were subsequently infected with LCMV Cl 13 (FIG. 7A). Surprisingly, P14 Csnk1a1 knockout P14 cells showed a reduction in overall cell numbers, Csnk1d knockout P14 cells had no change in cell numbers, and Csnk1g2 knockout P14 cells expanded significantly more than control cells by 8 days post-infection (FIG. 7B). This finding indicates that disruption of Csnk1g2 is beneficial for the T cell response because it leads to increased proliferation and/or survival of antigen-specific cells. Compared to control “scr” cells at 8 days post-infection, Csnk1g2 knockout P14 cells differentiated less frequently into SLAMF6+ TIM3− (T_{PROG}) or SLAMF6− TIM-3+ (encompassing several subsets more differentiated than T_{PROG}, including TEFF-LIKE and T_{TERM}). Instead, these Csnk1g2 knockout P14 cells preferentially differentiate into SLAMF6-TIM3− indicating that they are neither blocked at T_{PROG} nor terminally differentiating into TIM-3+ subsets (FIGS. 7C-7E). Compared to scr cells, Csnk1g2 knockout P14 cells express a similar amount of KLRG1, indicating that they are not preferentially differentiating into terminal effector (TE) cells common in acute infections²³ (FIG. 7F). However, Csnk1g2 knockout P14 cells produce significantly more proinflammatory cytokines upon restimulation than scr cells and express less of the TFs TOX and T-bet (FIGS. 7G-7I). These findings indicate that disruption of Csnk1g2 causes T cells to exhibit greater effector function and become less exhausted during chronic infection, while remaining uncommitted to a terminal effector fate. At 28 days post-infection with LCMV Cl 13, deletion of Csnk1g2 resulted in a substantial increase in cell counts, and a significantly greater effector function in the T_{TERM} subset (FIGS. 7J-7L). Together, these data show that disruption of Csnk1g2 in antigen-specific T cells leads to improvements in cell numbers and in effector function at multiple timepoints during the response to chronic viral infection.

Next, Csnk1g2 knockout P14 cells were assessed for efficacy in a tumor model. FIG. 7M shows an experimental schematic wherein B6 mice were implanted with B16-gp33 tumors on their flank. Csnk1g2 knockout P14 cells or scr cells were adoptively transferred 10 days post-implant, and tumors were collected on day 17 post-implant. Tumor size was measured by calipers throughout the experiment, and a growth curve of the tumor sizes for each genotype is shown in FIG. 7N. Tumor masses at endpoint are shown in FIG. 7O, and both the growth curves and the tumor masses show reductions in tumor size in mice that received Csnk1g2 knockout P14 cells. Tumor infiltrating P14 cells were profiled for PD-1 expression and Csnk1g2 knockout P14 cells showed a decreased level of PD-1 (FIG. 7P). Together, these data show that disruption of Csnk1g2 in antigen-specific T cells results in improved anti-tumor control.

These results show that the T cells with disrupted CK1G2 activity of the present technology are useful for driving the expansion and activity of T cell populations in a chronic infection model of T cell exhaustion and for treating cancer. Accordingly, the T cells comprising disrupted CK1G2 activity, and the agents used to disrupt CK1G2 activity, of the present disclosure are useful in compositions and methods for treating or preventing T cell exhaustion.

### Example 5: Deletion of the PRKCH or CSNK1G2 in Human GD2 CD8 CAR-T Cells Leads to an Improved Anti-Tumor Response Against Human U87 Tumors Implanted in NSG Mice

This example demonstrates that human T cells with disrupted PRKCH or CSNK1G2 gene activity have increased therapeutic efficacy against tumors in an in vivo model.

Example 4 demonstrates the efficacy of disrupting CSK1G2 gene activity in murine T cells to prevent T cell exhaustion in cancer and viral disease models. Further experiments were performed to confirm that the same efficacy would be observed with disruption of the equivalent human gene. As described above, primary human GD2-targeted CD8 CAR-T cells were modified using CRISPR/Cas-9 via lentiviral transduction to delete the PRKCH or CSNK1G2 genes, the equivalents to PKC eta and CK1G2 respectively in mice. See Overview section for sequence alignment comparison demonstrating high degree of cross-species sequence conservation. Human U87 tumors were then implanted into NSG mice and allowed to grow until palpable. 9×10⁵ GD2 CAR-T cells were administered at approximately 10 days post-implantation when tumors reached a mean volume of 50-70 mm³. Tumors were measured with calipers to calculate volume as described above.

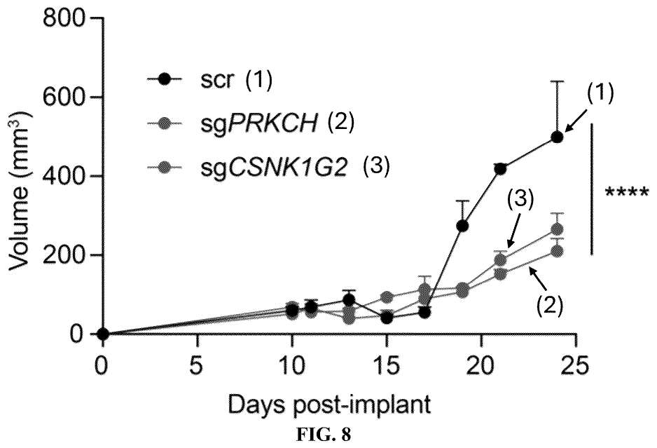

FIG. 8 shows a graph of tumor volume over time in mice treated with a control GD2 CD8 CAR-T cell line that received a scrambled CRISPR gRNA (scr), a PRKCH knockout GD2 CD8 CAR-T cell line (sgPRKCH), or a CSNK1G2 knockout GD2 CD8 CAR-T cell line (sgCSNK1G2). Disruption of PRKCH and CSNK1G2 activity in human CAR T cells resulted in a dramatic and significant increase in CAR T cell efficacy, as demonstrated by the reduction in tumor volume in treated mice compared to control mice.

These results show that the human T cells with disrupted CSNK1G2 or PRKCH activity of the present technology are useful for treating cancer. Accordingly, the T cells comprising disrupted CK1G2 activity, and the agents used to disrupt CK1G2 activity, of the present disclosure are useful in compositions and methods for treating or preventing T cell exhaustion.

### REFERENCES

1 Schmidt R, Ward C C, Dajani R, Armour-Garb Z, Ota M, Allain V, Hernandez R, Layeghi M, Xing G, Goudy L, Dorovskyi D, Wang C, Chen Y Y, Ye C J, Shy B R, Gilbert L A, Eyquem J, Pritchard J K, Dodgson S E, Marson A. Base-editing mutagenesis maps alleles to tune human T cell functions. Nature 2024; 625:805-12. https://doi.org/10.1038/s41586-023-06835-6.
2 Pear W S, Miller J P, Xu L, Pui J C, Soffer B, Quackenbush R C, Pendergast A M, Bronson R, Aster J C, Scott M L, Baltimore D. Efficient and rapid induction of a chronic myelogenous leukemia-like myeloproliferative disease in mice receiving p210 bcr/abl-transduced bone marrow. Blood 1998; 92:3780-92. https://doi.org/10.1182/blood.v92.10.3780.
3 Wu Y, Borde M, Heissmeyer V, Feuerer M, Lapan A D, Stroud J C, Bates D L, Guo L, Han A, Ziegler S F, Mathis D, Benoist C, Chen L, Rao A. FOXP3 Controls Regulatory T Cell Function through Cooperation with NFAT. Cell 2006; 126:375-87. https://doi.org/10.1016/j.cell.2006.05.042.
4 Demichev V, Messner C B, Vernardis S I, Lilley K S, Ralser M. DIA-NN: neural networks and interference correction enable deep proteome coverage in high throughput. NatMethods 2020; 17:41-4. https://doi.org/10.1038/s41592-019-0638-x.
5 Cox J, Hein M Y, Luber C A, Paron I, Nagaraj N, Mann M. Accurate proteome-wide label-free quantification by delayed normalization and maximal peptide ratio extraction, termed MaxLFQ. Mol Cell Proteomics 2014; 13:2513-26. https://doi.org/10.1074/mcp.M113.031591.
6 Johnson J L, Yaron T M, Huntsman E M, Kerelsky A, Song J, Regev A, Lin T-Y, Liberatore K, Cizin D M, Cohen B M, Vasan N, Ma Y, Krismer K, Robles J T, Kooij B van de, Vlimmeren A E van, Andree-Busch N, Kaufer N, Dorovkov M V., Ryazanov A G, Takagi Y, Kastenhuber E R, Goncalves M D, Elemento O, Taatjes D J, Maucuer A, Yamashita A, Degterev A, Linding R, Blenis J, Hornbeck P V., Turk B E, Yaffe M B, Cantley L C. A global atlas of substrate specificities for the human serine/threonine kinome. Nature 2023:2022.05.22.492882. https://doi.org/10.1038/s41586-022-05575-3.
7 Borrow P, Evans C F, Oldstone M B. Virus-induced immunosuppression: immune system-mediated destruction of virus-infected dendritic cells results in generalized immune suppression. J Virol 1995; 69:1059-70. https://doi.org/10.1128/jvi.69.2.1059-1070.1995.
8 Vardhana S A, Hwee M A, Berisa M, Wells D K, Yost K E, King B, Smith M, Herrera P S, Chang H Y, Satpathy A T, Brink M R M Van Den, Cross J R, Thompson C B. Impaired mitochondrial oxidative phosphorylation limits the self-renewal of T cells exposed to persistent antigen. Nat Immunol 2020; 21:1022-1033. https://doi.org/10.1038/s41590-020-0725-2.
9 Wu J E, Manne S, Ngiow S F, Baxter A E, Huang H, Freilich E, Clark M L, Lee J H, Chen Z, Khan O, Staupe R P, Huang Y J, Shi J, Giles J R, Wherry E J. In Vitro Modeling of CD8 T Cell Exhaustion Enables CRISPR Screening to Reveal a Role for BHLHE40. Sci Immunol 2023; 8:1-17. https://doi.org/10.1101/2023.04.17.537229.
10 Wippich F, Bodenmiller B, Trajkovska M G, Wanka S, Aebersold R, Pelkmans L. Dual specificity kinase DYRK3 couples stress granule condensation/dissolution to mTORCI signaling. Cell 2013; 152:791-805. https://doi.org/10.1016/j.cell.2013.01.033.
11 Volberding P J, Xin G, Kasmani M Y, Martinez E, Corbett J A, Cui W, Volberding P J, Xin G, Kasmani M Y, Khatun A, Brown A K, Nguyen C. Suppressive neutrophils require PIM1 for metabolic fitness and survival during chronic viral infection. Cell Reports 2021; 35:109160. https://doi.org/10.1016/j.celrep.2021.109160.
12 Gao Y, Bai F, Wang Y, Gao Y, Hu S, Li R, Jin S, Liu F, Liu X. Hyperprogression of cutaneous T cell lymphoma after anti-PD-1 treatment. JCI Insight 2023; 8.
13 Tripathi B K, Anderman M F, Bhargava D, Boccuzzi L, Qian X, Wang D, Durkin M E, Papageorge A G, Miguel F J De, Politi K, Walters K J, Doroshow J H, Lowy D R. Inhibition of cytoplasmic EZH2 induces antitumor activity through stabilization of the DLC1 tumor suppressor protein. Nat Commun 2021:1-15. https://doi.org/10.1038/s41467-021-26993-3.
14 Mayes-Hopfinger L, Enache A, Xie J, Huang C L, Kochl R, Tybulewicz V L J, Fernandes-Alnemri T, Alnemri E S. Chloride sensing by WNK1 regulates NLRP3 inflammasome activation and pyroptosis. Nat Commun 2021; 12:1-17. https://doi.org/10.1038/s41467-021-24784-4.
15 Dang F, Nie L, Zhou J, Shimizu K, Chu C, Wu Z, Fassl A, Ke S, Wang Y, Zhang J, Zhang T, Tu Z, Inuzuka H, Sicinski P, Bass A J, Wei W. Inhibition of CK1ε potentiates the therapeutic efficacy of CDK4/6 inhibitor in breast cancer. Nat Commun 2021; 12. https://doi.org/10.1038/s41467-021-25700-6.
16 Doench J G, Fusi N, Sullender M, Hegde M, Vaimberg E W, Donovan K F, Smith I, Tothova Z, Wilen C, Orchard R, Virgin H W, Listgarten J, Root D E. Optimized sgRNA design to maximize activity and minimize off-target effects of CRISPR-Cas9. Nat Biotechnol 2016; 34:184-91. https://doi.org/10.1038/nbt.3437.
17 Nüssing S, House I G, Kearney C J, Chen A X Y, Vervoort S J, Beavis P A, Oliaro J, Johnstone R W, Trapani J A, Parish I A. Efficient CRISPR/Cas9 Gene Editing in Uncultured Naive Mouse T Cells for In Vivo Studies. J Immunol 2020; 204:2308-15. https://doi.org/10.4049/jimmunol.1901396.
18 Kurachi M, Kurachi J, Chen Z, Johnson J, Khan O, Bengsch B, Stelekati E, Attanasio J, McLane L M, Tomura M, Ueha S, Wherry E J. Optimized retroviral transduction of mouse T cells for in vivo assessment of gene function. Nat Protoc 2017; 12:1980-98. https://doi.org/10.1038/nprot.2017.083.
19 Rape M. Post-Translational Modifications: Ubiquitylation at the crossroads of development and disease. Nat Rev Mol Cell Biol 2018; 19:59-70. https://doi.org/10.1038/nrm.2017.83.
20 Park J, Lee S Y, Jeon Y, Kim K M, Lee J K, Ko J, Park E J, Yoon J S, Kang B E, Ryu D, Lee H, Shin S J, Go H, Lee C W. The Pellinol-PKCu Signaling Axis Is an Essential Target for Improving Antitumor CD8b T-lymphocyte Function. Cancer Immunol Res 2022; 10:327-42. https://doi.org/10.1158/2326-6066.CIR-21-0419.
21 Baier-Bitterlich G, Uberall F, Bauer B, Fresser F, Wachter H, Grunicke H, Uterman G, Altman A, Baier G. Protein Kinase C-theta Isoenzyme Selective Stimulation of the Transcription Factor Complex AP-1 in T Lymphocytes. Mol Cell Biol 1996; 16:1842-50.
22 Xu Z B, Chaudhary D, Olland S, Wolfrom S, Czerwinski R, Malakian K, Lin L, Stahl M L, Joseph-McCarthy D, Benander C, Fitz L, Greco R, Somers W S, Mosyak L. Catalytic domain crystal structure of protein kinase C-θ (PKCθ). J Biol Chem 2004; 279:50401-9. https://doi.org/10.1074/jbc.M409216200.
23 Joshi N S, Cui W, Chandele A, Lee H K, Urso D R, Hagman J, Gapin L, Kaech S M. Inflammation Directs Memory Precursor and Short-Lived Effector CD8+ T Cell Fates via the Graded Expression of T-bet Transcription Factor. Immunity 2007; 27:281-95. https://doi.org/10.1016/j.immuni.2007.07.010.

1. An isolated nucleic acid molecule comprising a nucleotide sequence encoding a mutant protein kinase C (PKC) theta protein, wherein the nucleotide sequence is operably linked to a heterologous nucleic acid.

2. The isolated nucleic acid molecule of claim 1, wherein the heterologous nucleic acid comprises a promoter.

3. (canceled)

4. The isolated nucleic acid molecule of claim 1, wherein the mutant PKC theta protein comprises a K413R mutation.

5. The isolated nucleic acid molecule of claim 4, comprising a nucleotide sequence selected from the group consisting of:
(a) a nucleotide sequence set forth in SEQ ID NO: 1;
(b) a nucleotide sequence that encodes a polypeptide having the amino acid sequence set forth in SEQ ID NO: 2;
(c) a nucleotide sequence that is at least about 85% identical to the nucleotide sequences of (a) or (b), and which encodes a PKC theta protein comprising a K413R mutation.

6. The isolated nucleic acid molecule of claim 4, wherein the encoded mutant PKC theta protein is less susceptible to degradation compared to wild type PKC theta protein.

7. A plasmid comprising the isolated nucleic acid molecule of claim 1.

8.-9. (canceled)

10. A modified immune cell comprising the isolated nucleic acid molecule of claim 1.

11. The modified immune cell of claim 10, wherein the cell produces PKC theta K413R protein.

12.-18. (canceled)

19. A composition comprising the modified immune cell of claim 10.

20. A method of treating cancer in a subject in need thereof, comprising administering to the subject a therapeutically effective amount of the modified immune cell of claim 10.

21. The method of claim 20, wherein treatment of the subject results in the subject having one or more of:
(i) reduced levels of T cell exhaustion as compared to an untreated control subject with cancer
(ii) increased T cell proliferation;
(iii) increased T cell viability; or
(iv) increased T cell activity.

22.-37. (canceled)

38. A method of treating chronic microbial infection in a subject in need thereof, comprising administering to the subject a therapeutically effective amount of the modified immune cell of claim 10.

39.-60. (canceled)

61. A method for treating or preventing T cell exhaustion in a subject in need thereof, comprising administering an effective amount of one or more modified immune cells to a subject, wherein the one or more modified immune cells comprises a nucleic acid molecule comprising the nucleotide sequence selected from the group consisting of:
(a) a nucleotide sequence set forth in SEQ ID NO: 1;
(b) a nucleotide sequence that encodes a polypeptide having the amino acid sequence set forth in SEQ ID NO: 2; and
(c) a nucleotide sequence that is at least about 85% identical to the nucleotide sequences of (a) or (b), and which encodes a protein kinase C (PKC) theta protein comprising a K413R mutation.

62. The method of claim 61, wherein the subject has cancer.

63. (canceled)

64. The method of claim 61, wherein the subject is suffering from a microbial infection.

65.-91. (canceled)

92. A modified immune cell comprising disrupted CK1G2 activity, wherein the modified immune cell has been contacted with an agent that disrupts CK1G2 activity selected from the group consisting of: an antisense oligonucleotide, a shRNA, a siRNA, a zinc finger nuclease, and CRISPR-Cas system.

93. The modified immune cell of claim 92, wherein the modified immune cell produces less biologically active CK1G2 protein as compared to an untreated control cell.

94.-100. (canceled)

101. A composition comprising the modified immune cell of claim 92 and a pharmaceutically acceptable carrier.

102. A method of treating cancer in a subject in need thereof, comprising administering to the subject a therapeutically effective amount of the modified immune cell of claim 92.

103-119. (canceled)

120. A method of treating chronic microbial infection in a subject in need thereof, comprising administering to the subject a therapeutically effective amount of the modified immune cell of claim 92.

121.-219. (canceled)
